SEQUENCE LISTING (222 sequences)

SEQ ID NO: 1 - DNA, 1510 bp, Artificial sequence
  TRU-016 polynucleotide
aagcttgccgccatggattttcaagtgcagattttcagcttcctgctaatcagtgcttcagtcataattgccagaggagtcgacatccagatgactcagtctccagcctccctatctgcatctgtgggagagactgtcaccatcacatgtcgaacaagtgaaaatgtttacagttatttggcttggtatcagcagaaacagggaaaatctcctcagctcctggtctcttttgcaaaaaccttagcagaaggtgtgccatcaaggttcagtggcagtggatcaggcacacagttttctctgaagatcagcagcctgcagcctgaagattctggaagttatttctgtcaacatcattccgataatccgtggacgttcggtggaggcaccgaactggagatcaaaggtggcggtggctcgggcggtggtgggtcgggtggcggcggatcgtcagcggtccagctgcagcagtctggacctgagtcggaaaagcctggcgcttcagtgaagatttcctgcaaggcttctggttactcattcactggctacaatatgaactgggtgaagcagaataatggaaagagccttgagtggattggaaatattgatccttattatggtggtactacctacaaccggaagttcaagggcaaggccacattgactgtagacaaatcctccagcacagcctacatgcagctcaagagtctgacatctgaggactctgcagtctattactgtgcaagatcggtcggccctatggactactggggtcaaggaacctcagtcaccgtctcttcagatctggagcccaaatcttctgacaaaactcacacatctccaccgtgcccagcacctgaactcttgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtctaga

SEQ ID NO: 2 - protein, 496 aa, Artificial sequence
  TRU-016 polypeptide
MDFQVQIFSFLLISASVIIARGVDIQMTQSPASLSASVGETVTITCRTSENVYSYLAWYQQKQGKSPQLLVSFAKTLAEGVPSRFSGSGSGTQFSLKISSLQPEDSGSYFCQHHSDNPWTFGGGTELEIKGGGGSGGGGSGGGGSSAVQLQQSGPESEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSSDLEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 1518 bp, Artificial sequence
  TRU-015 polynucleotide
aagcttgccgccatggattttcaagtgcagattttcagcttcctgctaatcagtgcttcagtcataatgtccagaggacaaattgttctctcccagtctccagcaatcctgtctgcatctccaggggagaaggtcacaatgacttgcagggccagctcaagtgtaagttacatgcactggtaccagcagaagccaggatcctcccccaaaccctggatttatgccccatccaacctggcttctggagtccctgctcgcttcagtggcagtgggtctgggacctcttactctctcacaatcagcagagtggaggctgaagatgctgccacttattactgccagcagtggagttttaacccacccacgttcggtgctgggaccaagctggagctgaaagatggcggtggctcgggcggtggtggatctggaggaggtgggagctctcaggcttatctacagcagtctggggctgagtcggtgaggcctggggcctcagtgaagatgtcctgcaaggcttctggctacacatttaccagttacaatatgcactgggtaaagcagacacctagacagggcctggaatggattggagctatttatccaggaaatggtgatacttcctacaatcagaagttcaagggcaaggccacactgactgtagacaaatcctccagcacagcctacatgcagctcagcagcctgacatctgaagactctgcggtctatttctgtgcaagagtggtgtactatagtaactcttactggtacttcgatgtctggggcacagggaccacggtcaccgtctctgatcaggagcccaaatcttgtgacaaaactcacacatctccaccgtgctcagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatctaga

SEQ ID NO: 4 - protein, 499 aa, Artificial sequence
  TRU-015 polypeptide
MDFQVQIFSFLLISASVIMSRGQIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKDGGGSGGGGSGGGGSSQAYLQQSGAESVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSDQEPKSCDKTHTSPPCSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 1482 bp, Artificial sequence
  Humanized TRU-016 polynucleotide
atggaagccccagctcagcttctcttcctcctgctactctggctcccagataccaccggagaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtgggaccggtgaggtgcagctggtgcagtctggagcagaggtgaaaaagcccggagagtctctgaagatttcctgtaagggatccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 6 - protein, 493 aa, Artificial sequence
  Humanized TRU-016 polypeptide
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPMDYWGRGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 1482 bp, Artificial sequence
  Humanized TRU-016
atggaagccccagctcagcttctcttcctcctgctactctggctcccagataccaccggagaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtgggagctctgaggtgcagctggtgcagtctggagcagaggtgaaaaagcccggagagtctctgaagatttcctgtaagggatccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 8 - protein, 493 aa, Artificial sequence
  Humanized TRU-016
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGSSEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPMDYWGRGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 1482 bp, Artificial sequence
  Humanized TRU-016
atggaagccccagctcagcttctcttcctcctgctactctggctcccagataccaccggagaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtgggaccggtgaggtgcagctggtgcagtctggagcagagtcgaaaaagcccggagagtctctgaagatttcctgtaagggatccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 10 - protein, 493 aa, Artificial sequence
  Humanized TRU-016
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAESKKPGESLKISCKGSGYSFTGYNMNWVRQMPGKGLEWMGNIDPYYGGTTYNRKFKGQVTISADKSISTAYLQWSSLKASDTAMYYCARSVGPMDYWGRGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 1482 bp, Artificial sequence
  Humanized TRU-016
atggaagccccagctcagcttctcttcctcctgctactctggctcccagataccaccggagaaattgtgttgacacagtctccagccaccctgtctttgtctccaggcgaaagagccaccctctcctgccgaacaagtcaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtgggaccggtgaggtgcagctggtgcagtctggagcagaggtgaaaaagcccggagagtctctgaagatttcctgtaagggatccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 12 - protein, 493 aa, Artificial sequence
  Humanized TRU-016
MEAPAQLLFLLLLWLPDTTGEIVLTQSPATLSLSPGERATLSCRTSQNVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPMDYWGRGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 1482 bp, Artificial sequence
  Humanized TRU-016
atggaagccccagctcagcttctcttcctcctgctactctggctcccagataccaccggagaaattgtgttgacacagtctccagccaccctgtctttgtctccaggcgaaagagccaccctctcctgccgaacaagtgaaagtgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtgggaccggtgaggtgcagctggtgcagtctggagcagaggtgaaaaagcccggagagtctctgaagatttcctgtaagggatccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 14 - protein, 493 aa, Artificial sequence
  Humanized TRU-016
MEAPAQLLFLLLLWLPDTTGEIVLTQSPATLSLSPGERATLSCRTSESVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPMDYWGRGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 1482 bp, Artificial sequence
  Humanized TRU-016
atggaagccccagctcagcttctcttcctcctgctactctggctcccagataccaccggagaaattgtgttgacacagtctccagccaccctgtctttgtctccaggcgaaagagccaccctctcctgccgagcaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtgggaccggtgaggtgcagctggtgcagtctggagcagaggtgaaaaagcccggagagtctctgaagatttcctgtaagggatccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 16 - protein, 493 aa, Artificial sequence
  Humanized TRU-016
MEAPAQLLFLLLLWLPDTTGEIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPMDYWGRGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 1479 bp, Artificial sequence
  Humanized TRU-016
atggaagcaccagcgcagcttctcttcctcctgctactctggctcccagataccaccggtgacatccagatgactcagtctccagcctccctatctgcatctgtgggagagactgtcaccatcacatgtcgaacaagtgaaaatgtttacagttatttggcttggtatcagcagaaacagggaaaatctcctcagctcctggtctcttttgcaaaaaccttagcagaaggtgtgccatcaaggttcagtggcagtggatcaggcacacagttttctctgaagatcagcagcctgcagcctgaagattctggaagttatttctgtcaacatcattccgataatccgtggacgttcggtggaggcaccgaactggagatcaaaggtggcggtggctcgggcggtggtgggtcgggtggcggcggagctagcgaggtgcagctggtgcagtctggagcagaggtgaaaaagcccggagagtctctgaggatttcctgtaagggatccggttactcattcactggctacaatatgaactgggtgcgccagatgcccgggaaaggcctggagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctcgagcgagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 18 - protein, 492 aa, Artificial sequence
  Humanized TRU-016
MEAPAQLLFLLLLWLPDTTGDIQMTQSPASLSASVGETVTITCRTSENVYSYLAWYQQKQGKSPQLLVSFAKTLAEGVPSRFSGSGSGTQFSLKISSLQPEDSGSYFCQHHSDNPWTFGGGTELEIKGGGGSGGGGSGGGGASEVQLVQSGAEVKKPGESLRISCKGSGYSFTGYNMNWVRQMPGKGLEWMGNIDPYYGGTTYNRKFKGQVTISADKSISTAYLQWSSLKASDTAMYYCARSVGPMDYWGRGTLVTVSSSEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 1479 bp, Artificial sequence
  Humanized TRU-016
atggaagcaccagcgcagcttctcttcctcctgctactctggctcccagataccaccggtgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtagtggatctggaggaggtggagctagcgcggtccagctgcagcagtctggacctgagtcggaaaagcctggcgcttcagtgaagatttcctgcaaggcttctggttactcattcactggctacaatatgaactgggtgaagcagaataatggaaagagccttgagtggattggaaatattgatccttattatggtggtactacctacaaccggaagttcaagggcaaggccacattgactgtagacaaatcctccagcacagcctacatgcagctcaagagtctgacatctgaggactctgcagtctattactgtgcaagatcggtcggccctatggactactggggtcaaggaacctcagtcaccgtctcctcgagcgagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 20 - protein, 492 aa, Artificial sequence
  Humanized TRU-016
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGSGSGGGGASAVQLQQSGPESEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSSSEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 1479 bp, Artificial sequence
  Humanized TRU-016
atggaagcaccagcgcagcttctcttcctcctgctactctggctcccagataccaccggtgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtggagctagccaggtgcagctggtggagtctggtggaggcgtggtccagcctgggaggtccctgagactctcctgtgcagcctctggattcaccttcagtggctacaatatgaactgggtccgccagatgcccgggaaaggcctggagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctcgagcgagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 22 - protein, 492 aa, Artificial sequence
  Humanized TRU-016
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASQVQLVESGGGVVQPGRSLRLSCAASGFTFSGYNMNWVRQMPGKGLEWMGNIDPYYGGTTYNRKFKGQVTISADKSISTAYLQWSSLKASDTAMYYCARSVGPMDYWGRGTLVTVSSSEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - DNA, 1503 bp, Artificial sequence
  Humanized TRU-016
atggaagcaccagcgcagcttctcttcctcctgctactctggctcccagataccaccggtgcggtccagctgcagcagtctggacctgagtcggaaaagcctggcgcttcagtgaagatttcctgcaaggcttctggttactcattcactggctacaatatgaactgggtgaagcagaataatggaaagagccttgagtggattggaaatattgatccttattatggtggtactacctacaaccggaagttcaagggcaaggccacattgactgtagacaaatcctccagcacagcctacatgcagctcaagagtctgacatctgaggactctgcagtctattactgtgcaagatcggtcggccctatggactactggggtcaaggaacctcagtcaccgtctcttctggtggcggtggctcgggcggtggtgggtcgggtggcggcggatcaggaggaggcgggagtgctagcgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggctcgagcgagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 24 - protein, 500 aa, Artificial sequence
  Humanized TRU-016
MEAPAQLLFLLLLWLPDTTGAVQLQQSGPESEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSSGGGGSGGGGSGGGGSGGGGSASEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGSSEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - DNA, 1488 bp, Artificial sequence
  Humanized TRU-016
atggattttcaagtgcagattttcagcttcctgctaatcagtgcttcagtcataattgccagaggagtcgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtggagctagcgcggtccagctgcagcagtctggacctgagtcggaaaagcctggcgcttcagtgaagatttcctgcaaggcttctggttactcattcactggctacaatatgaactgggtgaagcagaataatggaaagagccttgagtggattggaaatattgatccttattatggtggtactacctacaaccggaagttcaagggcaaggccacattgactgtagacaaatcctccagcacagcctacatgcagctcaagagtctgacatctgaggactctgcagtctattactgtgcaagatcggtcggccctatggactactggggtcaaggaacctcagtcaccgtctcctcgagcgagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 26 - protein, 495 aa, Artificial sequence
  Humanized TRU-016
MDFQVQIFSFLLISASVIIARGVEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASAVQLQQSGPESEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSSSEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - DNA, 1503 bp, Artificial sequence
  Humanized TRU-016
atggaagcaccagcgcagcttctcttcctcctgctactctggctcccagataccaccggtgcggtccagctgcagcagtctggacctgagtcggaaaagcctggcgcttcagtgaagatttcctgcaaggcttctggttactcattcactggctacaatatgaactgggtgaagcagaataatggaaagagccttgagtggattggaaatattgatccttattatggtggtactacctacaaccggaagttcaagggcaaggccacattgactgtagacaaatcctccagcacagcctacatgcagctcaagagtctgacatctgaggactctgcagtctattactgtgcaagatcggtcggccctatggactactggggtcaaggaacctcagtcaccgtctcttctggtggcggtggctcgggcggtggtgggtcgggtggcggcggatcaggaggaggcgggagtgctagcgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggctcgagcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 28 - protein, 500 aa, Artificial sequence
  Humanized TRU-016
MEAPAQLLFLLLLWLPDTTGAVQLQQSGPESEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSSGGGGSGGGGSGGGGSGGGGSASEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - DNA, 1479 bp, Artificial sequence
  Humanized TRU-016
atggaagcaccagcgcagcttctcttcctcctgctactctggctcccagataccaccggtgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtggagctagcgcggtccagctgcagcagtctggacctgagtcggaaaagcctggcgcttcagtgaagatttcctgcaaggcttctggttactcattcactggctacaatatgaactgggtgaagcagaataatggaaagagccttgagtggattggaaatattgatccttattatggtggtactacctacaaccggaagttcaagggcaaggccacattgactgtagacaaatcctccagcacagcctacatgcagctcaagagtctgacatctgaggactctgcagtctattactgtgcaagatcggtcggccctatggactactggggtcaaggaacctcagtcaccgtctcctcgagcgagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 30 - protein, 492 aa, Artificial sequence
  Humanized TRU-016
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASAVQLQQSGPESEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSSSEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - DNA, 1479 bp, Artificial sequence
  Humanized TRU-016
atggaagcaccagcgcagcttctcttcctcctgctactctggctcccagataccaccggtgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtggagctagcgcggtccagctgcagcagtctggacctgagtcggaaaagcctggcgcttcagtgaagatttcctgcaaggcttctggttactcattcactggctacaatatgaactgggtgaagcagaataatggaaagagccttgagtggattggaaatattgatccttattatggtggtactacctacaaccggaagttcaagggcaaggccacattgactgtagacaaatcctccagcacagcctacatgcagctcaagagtctgacatctgaggactctgcagtctattactgtgcaagatcggtcggccctatggactactggggtcaaggaacctcagtcaccgtctcctcgagcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 32 - protein, 492 aa, Artificial sequence
  Humanized TRU-016
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASAVQLQQSGPESEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - DNA, 1479 bp, Artificial sequence
  Humanized TRU-016
atggaagcaccagcgcagcttctcttcctcctgctactctggctcccagataccaccggtgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtggagctagccaggtgcagctggtggagtctggtggaggcgtggtccagcctgggaggtccctgagactctcctgtgcagcctctggattcaccttcagtggctacaatatgaactgggtccgccagatgcccgggaaaggcctggagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctcgagcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 34 - protein, 492 aa, Artificial sequence
  Humanized TRU-016
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASQVQLVESGGGVVQPGRSLRLSCAASGFTFSGYNMNWVRQMPGKGLEWMGNIDPYYGGTTYNRKFKGQVTISADKSISTAYLQWSSLKASDTAMYYCARSVGPMDYWGRGTLVTVSSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - DNA, 1479 bp, Artificial sequence; identical to SEQ ID NO: 33
  Humanized TRU-016

SEQ ID NO: 36 - protein, 492 aa, Artificial sequence; identical to SEQ ID NO: 22
  Humanized TRU-016

SEQ ID NO: 37 - DNA, 1476 bp, Artificial sequence
  Humanized TRU-016
atggaagcaccagcgcagcttctcttcctcctgctactctggctcccagataccaccggtgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtggggctagcgaggtgcagctggtggagtctggtggaggcttggtccagcctggagggtccctgagactctcctgtgcagcctctggattcaccttcagtggctacaatatgaactgggtccgccagatgcccgggaaaggcctggagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctcgagcgagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 38 - protein, 492 aa, Artificial sequence
  Humanized TRU-016
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASEVQLVESGGGLVQPGGSLRLSCAASGFTFSGYNMNWVRQMPGKGLEWMGNIDPYYGGTTYNRKFKGQVTISADKSISTAYLQWSSLKASDTAMYYCARSVGPMDYWGRGTLVTVSSSEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - DNA, 1476 bp, Artificial sequence
  Humanized TRU-016
atggaagcaccagcgcagcttctcttcctcctgctactctggctcccagataccaccggtgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtggggctagcgaggtgcagctggtggagtctggtggaggcttggtccagcctggagggtccctgagactctcctgtgcagcctctggattcaccttcagtggctacaatatgaactgggtccgccagatgcccgggaaaggcctggagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctcgagcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 40 - protein, 492 aa, Artificial sequence
  Humanized TRU-016
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASEVQLVESGGGLVQPGGSLRLSCAASGFTFSGYNMNWVRQMPGKGLEWMGNIDPYYGGTTYNRKFKGQVTISADKSISTAYLQWSSLKASDTAMYYCARSVGPMDYWGRGTLVTVSSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - DNA, 1476 bp, Artificial sequence
  Humanized TRU-016
atggaagcaccagcgcagcttctcttcctcctgctactctggctcccagataccaccggtgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtggggctagcgaggtgcagctggtggagtctggtggaggctctgtccagcctggagggtccctgagactctcctgtgcagcctctggattcaccttcagtggctacaatatgaactgggtccgccagatgcccgggaaaggcctggagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctcgagcgagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 42 - protein, 492 aa, Artificial sequence
  Humanized TRU-016
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASEVQLVESGGGSVQPGGSLRLSCAASGFTFSGYNMNWVRQMPGKGLEWMGNIDPYYGGTTYNRKFKGQVTISADKSISTAYLQWSSLKASDTAMYYCARSVGPMDYWGRGTLVTVSSSEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - DNA, 1476 bp, Artificial sequence
  Humanized TRU-016
atggaagcaccagcgcagcttctcttcctcctgctactctggctcccagataccaccggtgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtggggctagcgaggtgcagctggtggagtctggtggaggctctgtccagcctggagggtccctgagactctcctgtgcagcctctggattcaccttcagtggctacaatatgaactgggtccgccagatgcccgggaaaggcctggagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctcgagcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 44 - protein, 492 aa, Artificial sequence
  Humanized TRU-016
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASEVQLVESGGGSVQPGGSLRLSCAASGFTFSGYNMNWVRQMPGKGLEWMGNIDPYYGGTTYNRKFKGQVTISADKSISTAYLQWSSLKASDTAMYYCARSVGPMDYWGRGTLVTVSSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - DNA, 1482 bp, Artificial sequence
  Humanized TRU-016
atggaagccccagctcagcttctcttcctcctgctactctggctcccagataccaccggagaaattgtgttgacacagtctccagccaccctgtctttgtctccaggcgaaagagccaccctctcctgccgagcaagtcaaagtgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtgggaccggtgaggtgcagctggtgcagtctggagcagaggtgaaaaagcccggagagtctctgaagatttcctgtaagggatccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 46 - protein, 493 aa, Artificial sequence
  Humanized TRU-016
MEAPAQLLFLLLLWLPDTTGEIVLTQSPATLSLSPGERATLSCRASQSVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPMDYWGRGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - DNA, 1500 bp, Artificial sequence
  Humanized TRU-016
aagcttgccgccatggaagccccagcgcagcttctcttcctcctgctactctggctcccagataccaccggagaaattgtgttgacacagtctccagccaccctgtctttgtctccaggcgaaagagccaccctctcctgccgagcaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggcggctcgggcggtggtggatctggaggaggtgggaccggtgaggtgcagctggtgcagtctggagcagaggtgaaaaagcccggagagtctctgaagatttcctgtaagggatccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctttcgactactggggccagggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatctaga

SEQ ID NO: 48 - protein, 493 aa, Artificial sequence
  Humanized TRU-016
MEAPAQLLFLLLLWLPDTTGEIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDYWGQGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - DNA, 356 bp, Artificial sequence
  Humanized TRU-016
gctagcgaggtgcagctggtgcagtctggagcagaggtgaaaaagcccggagagtctctgaagatttcctgtaagggatccggttactcattcactggctacaatatgaactgggtgcgccagatgcccgggaaaggcctggagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcgcgctcagtcggccctatggacgtctggggccaaggcaccactgtcactgtctcctcgag

SEQ ID NO: 50 - protein, 143 aa, Artificial sequence
  Humanized TRU-016
MEAPAQLLFLLLLWLPDTTGEIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTG

SEQ ID NO: 51 - DNA, 1381 bp, Artificial sequence
  Humanized TRU-016
aagcttgccgccatggaagccccagcgcagcttctcttcctcctgctactctggctcccagataccaccggagaaattgtgttgacacagtctccagccaccctgtctttgtctccaggcgaaagagccaccctctcctgccgagcaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtgggaccggtgaggtgcagctggtgcagtctggagcagaggtgaaaaagcccggagagtctctgaagatttcctgtaagggatccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctttcgactcctggggccagggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctca

SEQ ID NO: 52 - protein, 493 aa, Artificial sequence
  Humanized TRU-016
MEAPAQLLFLLLLWLPDTTGEIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDSWGQGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - DNA, 356 bp, Artificial sequence
  Humanized TRU-016
gctagcgaggtgcagctggtgcagtctggagcagaggtgaaaaagcccggagagtctctgaagatttcctgtaagggatccggttactcattcactggctacaatatgaactgggtgcgccagatgcccgggaaaggcctggagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcgcgctcagtcggcccttttgacccctggggccaaggcaccctggtcactgtctcctcgag

SEQ ID NO: 54 - protein, 143 aa, Artificial sequence; identical to SEQ ID NO: 50
  Humanized TRU-016

SEQ ID NO: 55 - DNA, 356 bp, Artificial sequence
  Humanized TRU-016
gctagcgaggtgcagctggtgcagtctggagcagaggtgaaaaagcccggagagtctctgaagatttcctgtaagggatccggttactcattcactggctacaatatgaactgggtgcgccagatgcccgggaaaggcctggagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcgcgctcagtcggcccttttcagcactggggccaaggcaccctcgtcactgtctcctcgag

SEQ ID NO: 56 - protein, 143 aa, Artificial sequence; identical to SEQ ID NO: 50
  Humanized TRU-016

SEQ ID NO: 57 - DNA, 356 bp, Artificial sequence
  Humanized TRU-016
gctagcgaggtgcagctggtgcagtctggagcagaggtgaaaaagcccggagagtctctgaagatttcctgtaagggatccggttactcattcactggctacaatatgaactgggtgcgccagatgcccgggaaaggcctggagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcgcgctcagtcggcccttttgacgtctggggccaaggcaccatggtcactgtctcctcgag

SEQ ID NO: 58 - protein, 143 aa, Artificial sequence; identical to SEQ ID NO: 50
  Humanized TRU-016

SEQ ID NO: 59 - DNA, 356 bp, Artificial sequence
  Humanized TRU-016
gctagcgaggtgcagctggtgcagtctggagcagaggtgaaaaagcccggagagtctctgaagatttcctgtaagggatccggttactcattcactggctacaatatgaactgggtgcgccagatgcccgggaaaggcctggagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcgcgctcagtcggcccttttgacatctggggccaaggcaccatggtcactgtctcctcgag

SEQ ID NO: 60 - protein, 143 aa, Artificial sequence; identical to SEQ ID NO: 50
  Humanized TRU-016

SEQ ID NO: 61 - protein, 11 aa, Artificial sequence
  Humanized TRU-016
RASENVYSYLA

SEQ ID NO: 62 - protein, 11 aa, Artificial sequence
  Humanized TRU-016
RTSENVYSYLA

SEQ ID NO: 63 - protein, 5 aa, Artificial sequence
  Humanized TRU-016
GYMNM

SEQ ID NO: 64 - protein, 7 aa, Artificial sequence
  Humanized TRU-016
FAKTLAE

SEQ ID NO: 65 - protein, 18 aa, Artificial sequence
  Humanized TRU-016
NIDPYYGGTTTYNRKFKG

SEQ ID NO: 66 - protein, 9 aa, Artificial sequence
  Humanized TRU-016
QHHSDNPWT

SEQ ID NO: 67 - protein, 7 aa, Artificial sequence
  Humanized TRU-016
SVGPFDY

SEQ ID NO: 68 - protein, 7 aa, Artificial sequence
  Humanized TRU-016
SVGPFDS

SEQ ID NO: 69 - protein, 7 aa, Artificial sequence
  Humanized TRU-016
SVGPMDY

SEQ ID NO: 70 - protein, 23 aa, Artificial sequence
  Humanized TRU-016
EIVLTQSPATLSLSPGERATLSC

SEQ ID NO: 71 - protein, 30 aa, Artificial sequence
  Humanized TRU-016
EVQLVQSGAEVKKPGESLKISCKGSGYSFT

SEQ ID NO: 72 - protein, 15 aa, Artificial sequence
  Humanized TRU-016
WYQQKPGQAPRLLIY

SEQ ID NO: 73 - protein, 14 aa, Artificial sequence
  Humanized TRU-016
WVRQMPGKGLEWMG

SEQ ID NO: 74 - protein, 32 aa, Artificial sequence
  Humanized TRU-016
GIPARFSGSGSGTDFTLTISSLEPEDFAVYYC

SEQ ID NO: 75 - protein, 32 aa, Artificial sequence
  Humanized TRU-016
QVTISADKSISTAYLQWSSLKASDTAMYYCAR

SEQ ID NO: 76 - protein, 10 aa, Artificial sequence
  Humanized TRU-016
FGQGTKVEIK

SEQ ID NO: 77 - protein, 11 aa, Artificial sequence
  Humanized TRU-016
WGQGTLVTVSS

SEQ ID NO: 78 - protein, 11 aa, Artificial sequence
  Humanized TRU-016
WGRGTLVTVSS

SEQ ID NO: 79 - DNA, 1500 bp, Artificial sequence
  Humanized TRU-016
aagcttgccgccatggaagccccagcgcagcttctcttcctcctgctactctggctcccagataccaccggagaaattgtgttgacacagtctccagccaccctgtctttgtctccaggcgaaagagccaccctctcctgccgagcaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtgggaccggtgaggtgcagctggtgcagtctggagcagaggtgaaaaagcccggagagtctctgaagatttcctgtaagggatccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctttcgacctctggggcagaggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatctaga

SEQ ID NO: 80 - protein, 493 aa, Artificial sequence
  Humanized TRU-016
MEAPAQLLFLLLLWLPDTTGEIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDLWGRGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - DNA, 1494 bp, Artificial sequence
  Humanized TRU-016
aagcttgccgccatggaagccccagctcagcttctcttcctcctgctactctggctcccagataccaccggagaaattgtgttgacacagtctccagccaccctgtctttgtctccaggcgaaagagccaccctctcctgccgagcaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtggggctagcgaggtgcagctggtgcagtctggagcagaggtgaaaaagcccggagagtctctgaagatttcctgtaagggatccggttactcattcactagctacaatatgaactgggtgcgccagatgcccgggaaaggcctggagtggatgggcaatattgatccttattatggtggtactaactacgcccagaagttccagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 82 - protein, 493 aa, Artificial sequence
  Humanized TRU-016
MEAPAQLLFLLLLWLPDTTGEIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASEVQLVQSGAEVKKPGESLKISCKGSGYSFTSYNMNWVRQMPGKGLEWMGNIDPYYGGTNYAQKFQGQVTISADKSISTAYLQWSSLKASDTAMYYCARSVGPMDYWGRGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - DNA, 1476 bp, Artificial sequence
  Humanized TRU-016
aagcttgccgccatggaagccccagcgcagcttctcttcctcctgctactctggctcccagataccaccggagaaattgtgttgacacagtctccagccaccctgtctttgtctccaggcgaaagagccaccctctcctgccgagcaagtgagaatgtttacagctacttagcctggtaccaacagaaacctggccaggctcctaggctcctcatctattttgcaaaaaccttagcagaagggattccagccagattcagtggcagtggttccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtgggagcggaggaggagctagcgaggtgcagctggtgcagtctggagcagaggtgaaaaagcccggagagtctctgaagatttcctgtaagggatccggttactcattcactggctacaatatgaactgggtgcgccagatgcccgggaaaggcctcgaatggatgggcaatattgatccttattatggtggtactacctacaaccggaagttcaagggccaggtcactatctccgccgacaagtccatcagcaccgcctacctgcaaggagcagcctgaaggcctcggacaccgccatgtattactgtgcacgctcagtcggccctttcgactcctggggccagggcaccctggtcactgtctcgagttgtccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactctaga

SEQ ID NO: 84 - protein, 485 aa, Artificial sequence
  Humanized TRU-016
MEAPAQLLFLLLLWLPDTTGEIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGSGGGASEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDSWGQGTLVTVSSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - DNA, 1494 bp, Artificial sequence
  Humanized TRU-016
aagcttgccgccatggaagccccagctcagcttctcttcctcctgctactctggctcccagataccaccggagaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtgggaccggtgaggtgcagctggtgcagtctggagcagaggtgaaaaagcccggagagtctctgaagatttcctgtaagggatccggttactcattcactggctacaatatgaactgggtgcgccagatgcccgggaaaggcctggagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 86 - protein, 493 aa, Artificial sequence
  Humanized TRU-016
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPMDYWGRGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - DNA, 1494 bp, Artificial sequence
  Humanized TRU-016
aagcttgccgccatggaagccccagctcagcttctcttcctcctgctactctggctcccagataccaccggtgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtggggctagcgaggtgcagctggtgcagtctggagcagaggtgaaaaagcccggagagtctctgaggatttcctgtaagggatccggttactcattcactggctacaatatgaactgggtgcgccagatgcccgggaaaggcctggagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 88 - protein, 493 aa, Artificial sequence
  Humanized TRU-016
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASEVQLVQSGAEVKKPGESLRISCKGSGYSFTGYNMNWVRQMPGKGLEWMGNIDPYYGGTTYNRKFKGQVTISADKSISTAYLQWSSLKASDTAMYYCARSVGPMDYWGRGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - DNA, 45 bp, Artificial sequence
  Humanized TRU-016
gagcccaaatcttgtgacaaaactcacacatgtccaccgtgccca

SEQ ID NO: 90 - protein, 15 aa, Artificial sequence
  Humanized TRU-016
EPKSCDKTHTCPPCP

SEQ ID NO: 91 - DNA, 45 bp, Artificial sequence
  Humanized TRU-016
gagcccaaatcttctgacaaaactcacacatgtccaccgtgccca

SEQ ID NO: 92 - protein, 15 aa, Artificial sequence
  Humanized TRU-016
EPKSSDKTHTCPPCP

SEQ ID NO: 93 - DNA, 45 bp, Artificial sequence
  Humanized TRU-016
gagcccaaatcttctgacaaaactcacacatgtccaccgtgctca

SEQ ID NO: 94 - protein, 15 aa, Artificial sequence
  Humanized TRU-016
EPKSSDKTHTCPPCS

SEQ ID NO: 95 - DNA, 45 bp, Artificial sequence
  Humanized TRU-016
gagcccaaatcttgtgacaaaactcacacatgtccaccgagctca

SEQ ID NO: 96 - protein, 15 aa, Artificial sequence
  Humanized TRU-016
EPKSSDKTHTCPPSS

SEQ ID NO: 97 - DNA, 45 bp, Artificial sequence
  Humanized TRU-016
gagcccaaatcttctgacaaaactcacacatctccaccgagccca

SEQ ID NO: 98 - protein, 15 aa, Artificial sequence
  Humanized TRU-016
EPKSSDKTHTSPPSP

SEQ ID NO: 99 - DNA, 45 bp, Artificial sequence
  Humanized TRU-016
gagcccaaatcttctgacaaaactcacacatctccaccgagctca

SEQ ID NO: 100 - protein, 15 aa, Artificial sequence
  Humanized TRU-016
EPKSSDKTHTSPPSS

SEQ ID NO: 101 - DNA, 45 bp, Artificial sequence
  Humanized TRU-016
gagcccaaatcttgtgacaaaactcacacatctccaccgtgccca

SEQ ID NO: 102 - protein, 15 aa, Artificial sequence
  Humanized TRU-016
EPKSCDKTHTSPPCP

SEQ ID NO: 103 - DNA, 45 bp, Artificial sequence
  Humanized TRU-016
gagcccaaatcttgtgacaaaactcacacatctccaccgtgctca

SEQ ID NO: 104 - protein, 15 aa, Artificial sequence
  Humanized TRU-016
EPKSCDKTHTSPPCS

SEQ ID NO: 105 - DNA, 45 bp, Artificial sequence
  Humanized TRU-016
gagcccaaatcttctgacaaaactcacacatctccaccgtgccca

SEQ ID NO: 106 - protein, 15 aa, Artificial sequence
  Humanized TRU-016
EPKSSDKTHTSPPCP

SEQ ID NO: 107 - DNA, 45 bp, Artificial sequence
  Humanized TRU-016
gagcccaaatcttctgacaaaactcacacatctccaccgtgctca

SEQ ID NO: 108 - protein, 15 aa, Artificial sequence
  Humanized TRU-016
EPKSSDKTHTSPPCS

SEQ ID NO: 109 - DNA, 45 bp, Artificial sequence
  Humanized TRU-016
gagcccaaatcttgtgacaaaactcacacatctccaccgagccca

SEQ ID NO: 110 - protein, 15 aa, Artificial sequence
  Humanized TRU-016
EPKSCDKTHTSPPSP

SEQ ID NO: 111 - DNA, 45 bp, Artificial sequence
  Humanized TRU-016
gagcccaaatcttgtgacaaaactcacacatctccaccgagctca

SEQ ID NO: 112 - protein, 15 aa, Artificial sequence
  Humanized TRU-016
EPKSCDKTHTSPPSS

SEQ ID NO: 113 - DNA, 45 bp, Artificial sequence; identical to SEQ ID NO: 95
  Humanized TRU-016

SEQ ID NO: 114 - protein, 15 aa, Artificial sequence
  Humanized TRU-016
EPKSSDKTHTCPPSS

SEQ ID NO: 115 - protein, 19 aa, Artificial sequence
  Humanized TRU-016
VPSTPPTPSPSTPPTPSPS

SEQ ID NO: 116 - protein, 6 aa, Artificial sequence
  Humanized TRU-016
VPPPPP

SEQ ID NO: 117 - DNA, 186 bp, Artificial sequence
  Humanized TRU-016
gagctcaaaactcctctcggggatacgacccatacgtgtccccgctgtcctgaaccgaagtcctgcgatacgcctccgccatgtccacggtgcccagagcccaaatcatgcgatacgcccccaccgtgtccccgctgtcctgaaccaaagtcatgcgataccccaccaccatgtccaagatgccca

SEQ ID NO: 118 - protein, 62 aa, Artificial sequence
  Humanized TRU-016
ELKTPLGDTTHTCPRCPEPKSCDTPPPCPRCPEPKSCDTPPPCPRCPEPKSCDTPPPCPRCP

SEQ ID NO: 119 - DNA, 45 bp, Artificial sequence
  Humanized TRU-016
gagcccaaatcttctgacacacctcccccatgcccacggtgcccc

SEQ ID NO: 120 - protein, 15 aa, Artificial sequence
  Humanized TRU-016
EPKSSDTPPPCPRCP

SEQ ID NO: 121 - DNA, 45 bp, Artificial sequence
  Humanized TRU-016
gagcccaaatcttgtgacacacctcccccatccccacggtcccca

SEQ ID NO: 122 - protein, 15 aa, Artificial sequence
  Humanized TRU-016
EPKSCDTPPPSPRSP

SEQ ID NO: 123 - DNA, 45 bp, Artificial sequence
  Humanized TRU-016
gagcccaaatcttctgacacacctcccccatccccacggtcccca

SEQ ID NO: 124 - protein, 15 aa, Artificial sequence
  Humanized TRU-016
EPKSSDTPPPSPRSP

SEQ ID NO: 125 - DNA, 45 bp, Artificial sequence
  Humanized TRU-016
gagcccaaatcttgtgacacacctcccccatccccacggtgccca

SEQ ID NO: 126 - protein, 15 aa, Artificial sequence
  Humanized TRU-016
EPKSCDTPPPSPRCP

SEQ ID NO: 127 - protein, 58 aa, Artificial sequence
  Humanized TRU-016
ESPKAQASSVPTAQPQAEGSLAKATTAPATTRNTGRGGEEKKKEKEKEEQEERETKTP

SEQ ID NO: 128 - protein, 11 aa, Artificial sequence
  Humanized TRU-016
RTSQNVYSYLA

SEQ ID NO: 129 - protein, 11 aa, Artificial sequence
  Humanized TRU-016
RTSESVYSYLA

SEQ ID NO: 130 - protein, 11 aa, Artificial sequence
  Humanized TRU-016
RASQSVYSYLA

SEQ ID NO: 131 - protein, 11 aa, Artificial sequence
  Humanized TRU-016
RASQSVSSYLA

SEQ ID NO: 132 - protein, 11 aa, Artificial sequence
  Humanized TRU-016
RASQSVSYYLA

SEQ ID NO: 133 - protein, 5 aa, Artificial sequence
  Humanized TRU-016
SYMNM

SEQ ID NO: 134 - protein, 5 aa, Artificial sequence
  Humanized TRU-016
SYWIG

SEQ ID NO: 135 - protein, 7 aa, Artificial sequence
  Humanized TRU-016
AASSLQS

SEQ ID NO: 136 - protein, 7 aa, Artificial sequence
  Humanized TRU-016
GASTRAT

SEQ ID NO: 137 - protein, 7 aa, Artificial sequence
  Humanized TRU-016
DASNRAT

SEQ ID NO: 138 - protein, 17 aa, Artificial sequence
  Humanized TRU-016
IIYPGDSDTRYSPSFQG

SEQ ID NO: 139 - protein, 17 aa, Artificial sequence
  Humanized TRU-016
RIDPSDSYTNYSPSFQG

SEQ ID NO: 140 - protein, 30 aa, Artificial sequence
  Humanized TRU-016
QVQLVQSGAEVKKPGASVKVSCKASGYTFT

SEQ ID NO: 141 - protein, 30 aa, Artificial sequence
  Humanized TRU-016
QVQLVQSGAEVKKPGSSVKVSCKASGGTFS

SEQ ID NO: 142 - protein, 30 aa, Artificial sequence; identical to SEQ ID NO: 141
  Humanized TRU-016

SEQ ID NO: 143 - protein, 30 aa, Artificial sequence
  Humanized TRU-016
EVQLVQSGAEVKKPGATVKISCKVSGYTFT

SEQ ID NO: 144 - protein, 30 aa, Artificial sequence; identical to SEQ ID NO: 71
  Humanized TRU-016

SEQ ID NO: 145 - protein, 30 aa, Artificial sequence
  Humanized TRU-016
EVQLVQSGAEVKKPGESLRISCKGSGYSFT

SEQ ID NO: 146 - protein, 30 aa, Artificial sequence
  Humanized TRU-016
QVQLVQSGSELKKPGASVKVSCKASGYTFT

SEQ ID NO: 147 - protein, 14 aa, Artificial sequence
  Humanized TRU-016
WVRQAPGQGLEWMG

SEQ ID NO: 148 - protein, 14 aa, Artificial sequence
  Humanized TRU-016
WVRQAPGQGLEWMG

SEQ ID NO: 149 - protein, 14 aa, Artificial sequence
  Humanized TRU-016
WVRQAPGQGLEWMG

SEQ ID NO: 150 - protein, 14 aa, Artificial sequence
  Humanized TRU-016
WVQQAPGKGLEWMG

SEQ ID NO: 151 - protein, 14 aa, Artificial sequence
  Humanized TRU-016
WVRQMPGKGLEWMG

SEQ ID NO: 152 - protein, 14 aa, Artificial sequence
  Humanized TRU-016
WVRQMPGKGLEWMG

SEQ ID NO: 153 - protein, 14 aa, Artificial sequence
  Humanized TRU-016
WVRQAPGQGLEWMG

SEQ ID NO: 154 - protein, 32 aa, Artificial sequence
  Humanized TRU-016
RVTMTTDTSTSTAYMELRSLRSDDTAVYYCAR

SEQ ID NO: 155 - protein, 32 aa, Artificial sequence
  Humanized TRU-016
RVTITADESTSTAYMELSSLRSEDTAVYYCAR

SEQ ID NO: 156 - protein, 32 aa, Artificial sequence
  Humanized TRU-016
RVTITADKSTSTAYMELSSLRSEDTAVYYCAR

SEQ ID NO: 157 - protein, 32 aa, Artificial sequence
  Humanized TRU-016
RVTITADTSTDTAYMELSSLRSEDTAVYYCAT

SEQ ID NO: 158 - protein, 32 aa, Artificial sequence; identical to SEQ ID NO: 75
  Humanized TRU-016

SEQ ID NO: 159 - protein, 32 aa, Artificial sequence
  Humanized TRU-016
HVTISADKSISTAYLQWSSLKASDTAMYYCAR

SEQ ID NO: 160 - protein, 32 aa, Artificial sequence
  Humanized TRU-016
RFVFSLDTSVSTAYLQISSLKAEDTAVYYCAR

SEQ ID NO: 161 - protein, 11 aa, Artificial sequence
  Humanized TRU-016
WGQGTLVTVSS

SEQ ID NO: 162 - protein, 11 aa, Artificial sequence
  Humanized TRU-016
WGRGTLVTVSS

SEQ ID NO: 163 - protein, 11 aa, Artificial sequence
  Humanized TRU-016
WGQGTMVTVSS

SEQ ID NO: 164 - protein, 11 aa, Artificial sequence
  Humanized TRU-016
WGQGTMVTVSS

SEQ ID NO: 165 - protein, 11 aa, Artificial sequence
  Humanized TRU-016
WGQGTLVTVSS

SEQ ID NO: 166 - protein, 11 aa, Artificial sequence
  Humanized TRU-016
WGQGTLVTVSS

SEQ ID NO: 167 - protein, 11 aa, Artificial sequence
  Humanized TRU-016
WGQGTLVTVSS

SEQ ID NO: 168 - protein, 11 aa, Artificial sequence
  Humanized TRU-016
WGQGTTVTVSS

SEQ ID NO: 169 - protein, 11 aa, Artificial sequence
  Humanized TRU-016
WGKGTTVTVSS

SEQ ID NO: 170 - protein, 23 aa, Artificial sequence
  Humanized TRU-016
EIVMTQSPATLSVSPGERATLSC

SEQ ID NO: 171 - protein, 23 aa, Artificial sequence
  Humanized TRU-016
EIVLTQSPATLSLSPGERATLSC

SEQ ID NO: 172 - protein, 23 aa, Artificial sequence
  Humanized TRU-016
DIQMTQSPSSLSASVGDRVTITC

SEQ ID NO: 173 - protein, 23 aa, Artificial sequence
  Humanized TRU-016
DIQMTQSPSSLSASVGDRVTITC

SEQ ID NO: 174 - protein, 23 aa, Artificial sequence
  Humanized TRU-016
DIQMTQSPSSLSASVGDRVTITC

SEQ ID NO: 175 - protein, 23 aa, Artificial sequence
  Humanized TRU-016
NIQMTQSPSAMSASVGDRVTITC

SEQ ID NO: 176 - protein, 23 aa, Artificial sequence
  Humanized TRU-016
DIQMTQSPSSLSASVGDRVTITC

SEQ ID NO: 177 - protein, 23 aa, Artificial sequence
  Humanized TRU-016
AIQLTQSPSSLSASVGDRVTITC

SEQ ID NO: 178 - protein, 23 aa, Artificial sequence
  Humanized TRU-016
DIQLTQSPSFLSASVGDRVTITC

SEQ ID NO: 179 - protein, 23 aa, Artificial sequence
  Humanized TRU-016
AIRMTQSPFSLSASVGDRVTITC

SEQ ID NO: 180 - protein, 23 aa, Artificial sequence
  Humanized TRU-016
AIQMTQSPSSLSASVGDRVTITC

SEQ ID NO: 181 - protein, 23 aa, Artificial sequence
  Humanized TRU-016
DIQMTQSPSTLSASVGDRVTITC

SEQ ID NO: 182 - protein, 15 aa, Artificial sequence
  Humanized TRU-016
WYQQKPGQAPRLLIY

SEQ ID NO: 183 - protein, 15 aa, Artificial sequence
  Humanized TRU-016
WYQQKPGQAPRLLIY

SEQ ID NO: 184 - protein, 15 aa, Artificial sequence
  Humanized TRU-016
WYQQKPGKAPKLLIY

SEQ ID NO: 185 - protein, 15 aa, Artificial sequence
  Humanized TRU-016
WYQQKPGKVPKLLIY

SEQ ID NO: 186 - protein, 15 aa, Artificial sequence
  Humanized TRU-016
WYQQKPGKAPKRLIY

SEQ ID NO: 187 - protein, 15 aa, Artificial sequence
  Humanized TRU-016
WFQQKPGKVPKHLIY

SEQ ID NO: 188 - protein, 15 aa, Artificial sequence
  Humanized TRU-016
WFQQKPGKAPKSLIY

SEQ ID NO: 189 - protein, 15 aa, Artificial sequence
  Humanized TRU-016
WYQQKPGKAPKLLIY

SEQ ID NO: 190 - protein, 15 aa, Artificial sequence
  Humanized TRU-016
WYQQKPGKAPKLLIY

SEQ ID NO: 191 - protein, 15 aa, Artificial sequence
  Humanized TRU-016
WYQQKPAKAPKLFIY

SEQ ID NO: 192 - protein, 15 aa, Artificial sequence
  Humanized TRU-016
WYQQKPGKAPKLLIY

SEQ ID NO: 193 - protein, 15 aa, Artificial sequence
  Humanized TRU-016
WYQQKPGKAPKLLIY

SEQ ID NO: 194 - protein, 32 aa, Artificial sequence
  Humanized TRU-016
GIPARFSGSGSGTEFTLTISSLQSEDFAVYYC

SEQ ID NO: 195 - protein, 32 aa, Artificial sequence; identical to SEQ ID NO: 74
  Humanized TRU-016

SEQ ID NO: 196 - protein, 32 aa, Artificial sequence
  Humanized TRU-016
GVPSRFSGSGSGTDFTLTISSLQPEDFATYYC

SEQ ID NO: 197 - protein, 32 aa, Artificial sequence
  Humanized TRU-016
GVPSRFSGSGSGTDFTLTISSLQPEDVATYYC

SEQ ID NO: 198 - protein, 32 aa, Artificial sequence
  Humanized TRU-016
GVPSRFSGSGSGTEFTLTISSLQPEDFATYYC

SEQ ID NO: 199 - protein, 32 aa, Artificial sequence; identical to SEQ ID NO: 198
  Humanized TRU-016

SEQ ID NO: 200 - protein, 32 aa, Artificial sequence; identical to SEQ ID NO: 196
  Humanized TRU-016

SEQ ID NO: 201 - protein, 32 aa, Artificial sequence; identical to SEQ ID NO: 196
  Humanized TRU-016

SEQ ID NO: 202 - protein, 32 aa, Artificial sequence; identical to SEQ ID NO: 198
  Humanized TRU-016

SEQ ID NO: 203 - protein, 32 aa, Artificial sequence
  Humanized TRU-016
GVPSRFSGSGSGTDYTLTISSLQPEDFATYYC

SEQ ID NO: 204 - protein, 32 aa, Artificial sequence; identical to SEQ ID NO: 196
  Humanized TRU-016

SEQ ID NO: 205 - protein, 32 aa, Artificial sequence
  Humanized TRU-016
GVPSRFSGSGSGTEFTLTISSLQPDDFATYYC

SEQ ID NO: 206 - protein, 10 aa, Artificial sequence
  Humanized TRU-016
FGQGTKVEIK

SEQ ID NO: 207 - protein, 10 aa, Artificial sequence
  Humanized TRU-016
FGQGTKLEIK

SEQ ID NO: 208 - protein, 10 aa, Artificial sequence
  Humanized TRU-016
FGPGTKVDIK

SEQ ID NO: 209 - protein, 10 aa, Artificial sequence
  Humanized TRU-016
FGGGTKVEIK

SEQ ID NO: 210 - protein, 10 aa, Artificial sequence
  Humanized TRU-016
FGQGTRLEIK

SEQ ID NO: 211 - protein, 7 aa, Artificial sequence
  Humanized TRU-016
SVGPMDY

SEQ ID NO: 212 - protein, 7 aa, Artificial sequence
  Humanized TRU-016
SVGPFDY

SEQ ID NO: 213 - protein, 7 aa, Artificial sequence
  Humanized TRU-016
SVGPMDV

SEQ ID NO: 214 - protein, 7 aa, Artificial sequence
  Humanized TRU-016
SVGPFDS

SEQ ID NO: 215 - protein, 7 aa, Artificial sequence
  Humanized TRU-016
SVGPFDP

SEQ ID NO: 216 - protein, 7 aa, Artificial sequence
  Humanized TRU-016
SVGPFQH

SEQ ID NO: 217 - protein, 7 aa, Artificial sequence
  Humanized TRU-016
SVGPFDV

SEQ ID NO: 218 - protein, 7 aa, Artificial sequence
  Humanized TRU-016
SVGPFDI

SEQ ID NO: 219 - protein, 7 aa, Artificial sequence
  Humanized TRU-016
SVGPFDL

SEQ ID NO: 220 - protein, 9 aa, Artificial sequence
  Humanized TRU-016
QHHSDNPWT

SEQ ID NO: 221 - DNA, 1530 bp, Artificial sequence
  TRU-016 polynucleotide
aagcttgccgccatggaagccccagctcagcttctcttcctcctgctactctggctcccagataccaccggagaggtgcagctggtgcagtctggagcagaggtgaaaaagcccggagagtctctgaagatttcctgtaagggctccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctttcgactcctggggccagggcaccctggtcactgtctcctctgggggtggaggctctggtggcggtggctctggcggaggtggatccggtggcggcggatctggcgggggtggctctgaaattgtgttgacacagtctccagccaccctgtctttgtctccaggcgaaagagccaccctctcctgccgagcaagtgaaaatgtttacagctacttagcctggtaccaacagaaacctggccaggctcctaggctcctcatctattttgcaaaaaccttagcagaaggaattccagccaggttcagtggcagtggctccgggacagacttcactctcaccatcagcagcctagagcctgaagattttgcagtttattactgtcaacatcattccgataatccgtggacattcggccaagggaccaaggtggaaatcaaaggtgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatctaga

SEQ ID NO: 222 - protein, 503 aa, Artificial sequence
  TRU-016 polypeptide
MEAPAQLLFLLLLWLPDTTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDSWGQGTLVTVSSGGGGSGGGGSGGGGSGGGGSGGGGSEIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2008/0279850 A1
(43) Pub. Date: Nov. 13, 2008

(54) B-Cell Reduction Using CD37-Specific and CD20-Specific Binding Molecules

(75) Inventors: William Brady, Bothell, WA (US); Martha S. Hayden-Ledbetter, Shoreline, WA (US); Jeffrey A. Ledbetter, Shoreline, WA (US); Sandy A. Simon, Seattle, WA (US); Peter A. Thompson, Bellevue, WA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: TRUBION PHARMACEUTICALS, INC., Seattle, WA (US)

(21) Appl. No.: 12/101,701

(22) Filed: Apr. 11, 2008

Related U.S. Application Data
(63) Continuation-in-part of application No. 11/493,132, filed on Jul. 25, 2006.
(60) Provisional application No. 60/702,499, filed on Jul. 25, 2005.
(60) Provisional application No. 60/800,595, filed on May 16, 2006.

(51) Int. Cl. A61K 39/395; C07K 16/18; C12N 15/11; C12N 15/00; C12N 5/06; C12P 21/04; A61P 37/00

(52) U.S. Cl. 424/133.1; 424/138.1; 530/387.3; 536/23.53; 435/320.1; 435/365; 435/69.1

(57) ABSTRACT

The present invention generally provides methods for B-cell reduction in an individual using CD37-specific binding molecules. In particular, the invention provides methods for B-cell reduction using CD37-specific binding molecules alone, or a combination of CD37-specific binding molecules and CD20-specific binding molecules, in some instances a synergistic combination. The invention further provides materials and methods for treatment of diseases involving aberrant B-cell activity. In addition, the invention provides humanized CD37-specific binding molecules.

The present application is a continuation-in-part of U.S. patent application Ser. No. 11/493,132, which was filed Jul. 25, 2006, which claims benefit under 35 U.S.C. § 119 of U.S. Patent Application No. 60/702,499, which was filed Jul. 25, 2005, and U.S. Patent Application No. 60/800,595, which was filed May 16, 2006, each of which is incorporated herein by reference in its entirety.

### FIELD OF THE INVENTION

The present invention generally provides methods for B-cell reduction in an individual using CD37-specific binding molecules. In particular, the invention provides methods for B-cell reduction using CD37-specific binding molecules alone, or a combination of CD37-specific binding molecules and CD20-specific binding molecules, in some instances a synergistic combination. The invention further provides materials and methods for treatment of diseases involving aberrant B-cell activity.

### BACKGROUND OF THE INVENTION

In its usual role, the human immune system protects the body from damage from foreign substances and pathogens. One way in which the immune system protects the body is by production of specialized cells called B lymphocytes or B-cells. B-cells produce antibodies that bind to, and in some cases mediate destruction of, a foreign substance or pathogen.

In some instances though, the human immune system and specifically the B lymphocytes of the human immune system go awry and disease results. There are numerous cancers that involve uncontrolled proliferation of B-cells. There are also numerous autoimmune diseases that involve B-cell production of antibodies that, instead of binding to foreign substances and pathogens, bind to parts of the body. Such antibodies are sometimes called autoantibodies. In addition, there are numerous autoimmune and inflammatory diseases that involve B-cells in their pathology, for example, through inappropriate B-cell antigen presentation to T-cells, or through other pathways involving B-cells. For example, autoimmune-prone mice deficient in B-cells do not develop autoimmune kidney disease, vasculitis or autoantibodies. See Shlomchik et al., J. Exp. Med., 180:1295-306 (1994). Interestingly, these same autoimmune-prone mice which possess B-cells but are deficient in immunoglobulin production, do develop autoimmune diseases when induced experimentally as described by Chan et al., J. Exp. Med., 189:1639-48 (1999), indicating that B-cells play an integral role in development of autoimmune disease.

B-cells can be identified by molecules on their cell surface. CD20 was the first human B-cell lineage-specific surface molecule identified by a monoclonal antibody. It is a non-glycosylated, hydrophobic 35 kDa B-cell transmembrane phosphoprotein that has both its amino and carboxy ends situated inside the cell. See, Einfeld et al., EMBO J., 7:711-17 (1998). CD20 is expressed by all normal mature B-cells, but is not expressed by precursor B-cells or plasma cells. Natural ligands for CD20 have not been identified, and the function of CD20 in B-cell biology is still incompletely understood.

Another B-cell lineage-specific cell surface molecule is CD37. CD37 is a heavily glycosylated 40-52 kDa protein that belongs to the tetraspanin transmembrane family of cell surface antigens. It traverses the cell membrane four times forming two extracellular loops and exposing its amino and carboxy ends to the cytoplasm. CD37 is highly expressed on normal antibody-producing (slg+)B-cells, but is not expressed on pre-B-cells or plasma cells. The expression of CD37 on resting and activated T cells, monocytes and granulocytes is low and there is no detectable CD37 expression on NK cells, platelets or erythrocytes. See, Belov et al., Cancer Res., 61(11):4483-4489 (2001); Schwartz-Albiez et al., J. Immunol., 140(3): 905-914 (1988); and Link et al., J. Immunol., 137(9): 3013-3018 (1988). Besides normal B-cells, almost all malignancies of B-cell origin are positive for CD37 expression, including CLL, NHL, and hairy cell leukemia [Moore et al., Journal of Pathology, 152: 13-21 (1987); Merson and Brochier, Immunology Letters, 19: 269-272 (1988); and Faure et al., American Journal of Dermatopathology, 12 (3): 122-133 (1990)]. CD37 participates in regulation of B-cell function, since mice lacking CD37 were found to have low levels of serum IgG1 and to be impaired in their humoral response to viral antigens and model antigens. It appears to act as a nonclassical costimulatory molecule or by directly influencing antigen presentation via complex formation with MHC class II molecules. See Knobeloch et al., Mol. Cell. Biol., 20(15):5363-5369 (2000). CD37 also seems to play a role in TCR signaling. See Van Spriel et al., J. Immunol., 172: 2953-2961 (2004).

Research and drug development has occurred based on the concept that B-cell lineage-specific cell surface molecules such as CD37 or CD20 can themselves be targets for antibodies that would bind to, and mediate destruction of, cancerous and autoimmune disease-causing B-cells that have CD37 or CD20 on their surfaces. Termed “immunotherapy,” antibodies made (or based on antibodies made) in a non-human animal that bind to CD37 or CD20 were given to a patient to deplete cancerous or autoimmune disease-causing B-cells.

One antibody to CD37 has been labeled with ¹³¹I and tested in clinical trials for therapy of NHL. See Press et al., J. Clin. Oncol., 7(3): 1027-1038 (1989); Bernstein et al., Cancer Res. (Suppl.), 50: 1017-1021 (1990); Press et al., Front. Radiat. Ther. Oncol., 24: 204-213 (1990); Press et al., Adv. Exp. Med. Biol., 303: 91-96 (1991) and Brown et al., Nucl. Med. Biol., 24: 657-663 (1997). The antibody, MB-1, is a murine IgG1 monoclonal antibody that lacks Fc effector functions such as antibody-dependent cellular cytotoxicity (ADCC) and MB-1 did not inhibit tumor growth in an in vivo xenograft model unless it had been labeled with an isotope (Buchsbaum et al., Cancer Res., 52(83): 6476-6481 (1992). Favorable biodistribution of ¹³¹I-MB-1 was seen in lymphoma patients who had lower tumor burdens (<1 kg) and therapy of these patients resulted in complete tumor remissions lasting from 4 to 11 months (Press et al., 1989 and Bernstein et al. 1990).

In addition, an immunoconjugate composed of the drug adriamycin linked to G28-1, another anti-CD37 antibody, has been evaluated in mice and showed effects through internalization and intracellular release of the drug. See Braslawsky et al., Cancer Immunol. Immunother., 33(6): 367-374 (1991).

Various groups have investigated the use of anti-CD20 antibodies to treat B-cell related diseases. One treatment consists of anti-CD20 antibodies prepared in the form of radionuclides for treating B-cell lymphoma (e.g., ¹³¹I-labeled anti-CD20 antibody), as well as a ⁸⁹Sr-labeled form for the palliation of bone pain caused by prostate and breast cancer metastases [Endo, Gan To Kagaku Ryoho, 26: 744-748 (1999)].

Another group developed a chimeric monoclonal antibody specific for CD20, consisting of heavy and light chain variable regions of mouse origin fused to human IgG1 heavy chain and human kappa light chain constant regions. The chimeric antibody reportedly retained the ability to bind to CD20 and the ability to mediate ADCC and to fix complement. See, Liu et al., J. Immunol. 139:3521-26 (1987). Yet another chimeric anti-CD20 antibody was made from IDEC hybridoma C2B8 and was named rituximab. The mechanism of anti-tumor activity of rituximab is thought to be a combination of several activities, including ADCC, complement fixation, and triggering of signals that promote apoptosis in malignant B-cells, although the large size of the chimeric antibody prevents optimal diffusion of the molecule into lymphoid tissues that contain malignant B-cells, thereby limiting its anti-tumor activities. ADCC is a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. Complement fixation, or complement-dependent cytotoxicity (CDC) is the ability of a molecule to lyse a target in the presence of complement. The complement activation pathway is initiated by the binding of the first component of the complement system (C1q) to a molecule (e.g. an antibody) complexed with a cognate antigen. The large size of rituximab prevents optimal diffusion of the molecule into lymphoid tissues that contain malignant B-cells, thereby limiting these anti-tumor activities.

Rituximab, typically administered in 4 weekly infusions, is currently used to treat low-grade or follicular B-cell non-Hodgkin's lymphoma [McLaughlin et al., Oncology, 12: 1763-1777 (1998); Leget et al., Curr. Opin. Oncol., 10: 548-551 (1998)] and in relapsed stage III/IV follicular lymphoma [White et al., Pharm. Sci. Technol. Today, 2: 95-101 (1999)]. Other disorders treatable with rituximab include follicular centre cell lymphoma (FCC), mantle cell lymphoma (MCL), diffuse large cell lymphoma (DLCL), and small lymphocytic lymphoma (SLL) [Nguyen et al., Eur J. Haematol., 62:76-82 (1999)]. Rituximab administered in weekly infusions is also used to treat CLL [Lin et al., Sem Oncol., 30:483-92 (2003)].

Anti-CD20 antibodies have also been used to treat patients suffering from autoimmune diseases associated with B-cell production of autoantibodies. For example, rituximab has demonstrated significant clinical benefit in depleting CD20+ B-cells in patients with multiple autoimmune/inflammatory diseases including RA [Edwards, N Engl J. Med., 350:2546-2548 (2004); Cambridge et al., Arthritis Rheum., 48:2146-54 (2003)]. RA patients received continued doses of methotrexate (MTX) and a 4 dose course of rituximab infusion (Edwards, supra). These patients showed improved American College of Rheumatology (ACR) responses compared to control groups.

In a trial for the treatment of systemic lupus erythematosus (SLE) [Leandro et al., Arthritis Rheum., 46:2673-2677 (2002)], patients were administered two infusions of high dose rituximab, and demonstrated B-cell reduction and improved disease state. In a second study of B-cell reduction in SLE [Looney et al., Arthritis Rheum., 50:2580-2589 (2004)], patients were given a single infusion of 100 mg/m2 (low dose), a single infusion of 375 mg/m2 (intermediate dose), or as 4 infusions (1 week apart) of 375 mg/m2 (high dose) rituximab. These patients demonstrated B-cell reduction and improved disease scores, but the treatment did not alter the level of autoantibody. Trials of rituximab have also been carried out in Waldenstrom's macroglobulinemia [Treon et al., Immunother., 24:272-279 (2000)], where patients showed increased hematocrit (HCT) and platelet (PLT) counts after 4 infusions of rituximab.

Recent reports of rituximab treatment in patients suffering from multiple sclerosis, an autoimmune disease affecting the central nervous system, indicate that a course of treatment depletes peripheral B-cells but has little effect on B-cells in cerebrospinal fluid. See Monson et al., Arch. Neurol., 62: 258-264 (2005).

Additional publications concerning the use of rituximab include: Stashi et al. “Rituximab chimeric anti-CD20 monoclonal antibody treatment for adults with chronic idiopathic thrombocytopenic purpura” Blood 98:952-957 (2001); Matthews, R. “Medical Heretics” New Scientist (7 Apr., 2001); Leandro et al. “Clinical outcome in 22 patients with rheumatoid arthritis treated with B lymphocyte depletion” Ann Rheum Dis 61:833-888 (2002); Leandro et al. “Lymphocyte depletion in rheumatoid arthritis: early evidence for safety, efficacy and dose response. Arthritis and Rheumatism 44(9): S370 (2001); Leandro et al. “An open study of B lymphocyte depletion in systemic lupus erythematosus”, Arthritis Rheum. 46:2673-2677 (2002); Edwards et al., “Sustained improvement in rheumatoid arthritis following a protocol designed to deplete B lymphocytes” Rheumatology 40:205-211 (2001); Edwards et al. “B-lymphocyte depletion therapy in rheumatoid arthritis and other autoimmune disorders” Biochem. Soc. Trans. 30(4):824-828 (2002); Edwards et al. “Efficacy and safety of rituximab, a B-cell targeted chimeric monoclonal antibody: A randomized, placebo controlled trial in patients with rheumatoid arthritis. Arthritis Rheum. 46: S197 (2002); Levine et al., “IgM antibody-related polyneuropathies: B-cell depletion chemotherapy using rituximab” Neurology 52: 1701-1704 (1999); DeVita et al. “Efficacy of selective B-cell blockade in the treatment of rheumatoid arthritis” Arthritis Rheum 46:2029-2033 (2002); Hidashida et al. “Treatment of DMARD-Refractory rheumatoid arthritis with rituximab.” Presented at the Annual Scientific Meeting of the American College of Rheumatology; October 24-29; New Orleans, La. 2002; Tuscano, J. “Successful treatment of Infliximab-refractory rheumatoid arthritis with rituximab” Presented at the Annual Scientific Meeting of the American College of Rheumatology; October 24-29; New Orleans, La. 2002.

Problems associated with rituximab therapy remain. For example, the majority of cancer patients treated with rituximab relapse, generally within about 6-12 months, and fatal infusion reactions within 24 hours of rituximab infusion have been reported. These fatal reactions followed an infusion reaction complex that included hypoxia, pulmonary infiltrates, acute respiratory distress syndrome, myocardial infarction, ventricular fibrillation or cardiogenic shock. Acute renal failure requiring dialysis with instances of fatal outcome has also been reported in the setting of tumor lysis syndrome following treatment with rituximab, as have severe mucocutaneous reactions, some with fatal outcome. Additionally, high doses of rituximab are required for intravenous injection because the molecule is large, approximately 150 kDa, and, as noted above, diffusion into the lymphoid tissues where many tumor cells reside is limited.

Because normal mature B-cells also express CD37 and CD20, normal B-cells are depleted by anti-CD37 (Press et al., 1989) or anti-CD20 antibody therapy [Reff et al., Blood, 83:435-445 (1994)]. After treatment is completed, however, normal B-cells can be regenerated from CD37- and CD20-negative B-cell precursors; therefore, patients treated with anti-CD37 or anti-CD20 therapy do not experience significant immunosuppression.

Monoclonal antibody technology and genetic engineering methods have led to development of immunoglobulin molecules for diagnosis and treatment of human diseases. Protein engineering has been applied to improve the affinity of an antibody for its cognate antigen, to diminish problems related to immunogenicity, and to alter an antibody's effector functions. The domain structure of immunoglobulins is amenable to engineering, in that the antigen binding domains and the domains conferring effector functions may be exchanged between immunoglobulin classes and subclasses. Immunoglobulin structure and function are reviewed, for example, in Harlow et al., Eds., Antibodies: A Laboratory Manual, Chapter 14, Cold Spring Harbor Laboratory, Cold Spring Harbor (1988). An extensive introduction as well as detailed information about all aspects of recombinant antibody technology can be found in the textbook “Recombinant Antibodies” (John Wiley & Sons, NY, 1999). A comprehensive collection of detailed antibody engineering lab Protocols can be found in R. Kontermann and S. Dübel (eds.), “The Antibody Engineering Lab Manual” (Springer Verlag, Heidelberg/N.Y., 2000).

Recently, smaller immunoglobulin molecules have been constructed to overcome problems associated with whole immunoglobulin therapy. Single chain Fv (scFv) comprise an antibody heavy chain variable domain joined via a short linker peptide to an antibody light chain variable domain [Huston et al., Proc. Natl. Acad. Sci. USA, 85: 5879-5883 (1988)]. In addition to variable regions, each of the antibody chains has one or more constant regions. Light chains have a single constant region domain. Thus, light chains have one variable region and one constant region. Heavy chains have several constant region domains. The heavy chains in IgG, IgA, and IgD antibodies have three constant region domains, which are designated CH1, CH2, and CH3, and the heavy chains in IgM and IgE antibodies have four constant region domains, CH1, CH2, CH3 and CH4. Thus, heavy chains have one variable region and three or four constant regions.

The heavy chains of immunoglobulins can also be divided into three functional regions: the Fd region (a fragment comprising V.sub.H and CH₁, i.e., the two N-terminal domains of the heavy chain), the hinge region, and the Fc region (the “fragment crystallizable” region, derived from constant regions and formed after pepsin digestion). The Fd region in combination with the light chain forms an Fab (the “fragment antigen-binding”). Because an antigen will react stereochemically with the antigen-binding region at the amino terminus of each Fab the IgG molecule is divalent, i.e., it can bind to two antigen molecules. The Fc contains the domains that interact with immunoglobulin receptors on cells and with the initial elements of the complement cascade. Thus, the Fc fragment is generally considered responsible for the effector functions of an immunoglobulin, such as complement fixation and binding to Fc receptors.

Because of the small size of scFv molecules, they exhibit very rapid clearance from plasma and tissues and more effective penetration into tissues than whole immunoglobulin. An anti-tumor scFv showed more rapid tumor penetration and more even distribution through the tumor mass than the corresponding chimeric antibody [Yokota et al., Cancer Res., 52, 3402-3408 (1992)]. Fusion of an scFv to another molecule, such as a toxin, takes advantage of the specific antigen-binding activity and the small size of an scFv to deliver the toxin to a target tissue. [Chaudary et al., Nature, 339:394 (1989); Batra et al., Mol. Cell. Biol., 11:2200 (1991)].

Despite the advantages of scFv molecules, several drawbacks to their use exist. While rapid clearance of scFv may reduce toxic effects in normal cells, such rapid clearance may prevent delivery of a minimum effective dose to the target tissue. Manufacturing adequate amounts of scFv for administration to patients has been challenging due to difficulties in expression and isolation of scFv that adversely affect the yield. During expression, scFv molecules lack stability and often aggregate due to pairing of variable regions from different molecules. Furthermore, production levels of scFv molecules in mammalian expression systems are low, limiting the potential for efficient manufacturing of scFv molecules for therapy [Davis et al, J. Biol. Chem., 265:10410-10418 (1990); Traunecker et al., EMBO J, 10: 3655-3659 (1991). Strategies for improving production have been explored, including addition of glycosylation sites to the variable regions [Jost, C. R. U.S. Pat. No. 5,888,773, Jost et al, J. Biol. Chem., 69: 26267-26273 (1994)].

Another disadvantage to using scFv for therapy is the lack of effector function. An scFv without the cytolytic functions, ADCC and complement dependent-cytotoxicity (CDC), associated with the constant region of an immunoglobulin may be ineffective for treating disease. Even though development of scFv technology began over 12 years ago, currently no scFv products are approved for therapy.

Alternatively, it has been proposed that fusion of an scFv to another molecule, such as a toxin, could take advantage of the specific antigen-binding activity and the small size of an scFv to deliver the toxin to a target tissue. Chaudary et al., Nature 339:394 (1989); Batra et al., Mol. Cell. Biol. 11:2200 (1991). Conjugation or fusion of toxins to scFvs has thus been offered as an alternative strategy to provide potent, antigen-specific molecules, but dosing with such conjugates or chimeras can be limited by excessive and/or non-specific toxicity due to the toxin moiety of such preparations. Toxic effects may include supraphysiological elevation of liver enzymes and vascular leak syndrome, and other undesired effects. In addition, immunotoxins are themselves highly immunogenic upon administration to a host, and host antibodies generated against the immunotoxin limit potential usefulness for repeated therapeutic treatments of an individual.

Other engineered fusion proteins, termed small, modular immunopharmaceutical (SMIP™) products, are described in co-owned US Patent Publications 2003/133939, 2003/0118592, and 2005/0136049, and co-owned International Patent Publications WO02/056910, WO2005/037989., and WO2005/017148, which are all incorporated by reference herein. SMIP products are novel binding domain-immunoglobulin fusion proteins that feature a binding domain for a cognate structure such as an antigen, a counterreceptor or the like; an IgG1, IGA or IgE hinge region polypeptide or a mutant IgG1 hinge region polypeptide having either zero, one or two cysteine residues; and immunoglobulin CH2 and CH3 domains. SMIP products are capable of ADCC and/or CDC.

Although there has been extensive research carried out on antibody-based therapies, there remains a need in the art for improved methods to treat diseases associated with aberrant B-cell activity. The methods of the present invention described and claimed herein provide such improved methods as well as other advantages.

### SUMMARY OF THE INVENTION

The present invention provides methods for reducing B-cells using CD37-specific binding molecules. In some methods of the invention, use of combinations of CD37-specific binding molecules (one or more CD37-specific binding molecules) and CD20-specific binding molecules (one or more CD20-specific binding molecules) results in increased B-cell reduction. In some of these methods, the combinations are synergistic. In a related aspect, the invention provides a method of treating an individual having, or suspected of having, a disease associated with aberrant B-cell activity.

The present invention also provides humanized CD37-specific binding molecules (e.g., humanized TRU-016 constructs) and methods for reducing B-cells using these molecules. In some embodiments of the methods of the invention, uses of combinations of humanized TRU-016 constructs with one or more CD20-specific binding molecules is contemplated. In another aspect, the invention provides methods of treating individuals having, or suspected of having, a disease associated with aberrant B-cell activity. Related aspects of the invention are drawn to methods of preventing any such disease and methods of ameliorating a symptom associated with such a disease comprising administering a dose of a humanized CD37-specific binding molecule effective to treat or prevent such disease, or to ameliorate a symptom of such disease.

“Aberrant B-cell activity” refers to B-cell activity that deviates from the normal, proper, or expected course. For example, aberrant B-cell activity may include inappropriate proliferation of cells whose DNA or other cellular components have become damaged or defective. Aberrant B-cell activity may include cell proliferation whose characteristics are associated with a disease caused by, mediated by, or resulting in inappropriately high levels of cell division, inappropriately low levels of apoptosis, or both. Such diseases may be characterized, for example, by single or multiple local abnormal proliferations of cells, groups of cells or tissue(s), whether cancerous or non-cancerous, benign or malignant. Aberrant B-cell activity may also include aberrant antibody production, such as production of autoantibodies, or overproduction of antibodies typically desirable when produced at normal levels. It is contemplated that aberrant B-cell activity may occur in certain subpopulations of B-cells and not in other subpopulations. Aberrant B-cell activity may also include inappropriate stimulation of T-cells, such as by inappropriate B-cell antigen presentation to T-cells or by other pathways involving B-cells.

“Treatment” or “treating” refers to either a therapeutic treatment or prophylactic/preventative treatment. A therapeutic treatment may improve at least one symptom of disease in an individual receiving treatment or may delay worsening of a progressive disease in an individual, or prevent onset of additional associated diseases.

A “therapeutically effective dose” or “effective dose” of a specific binding molecule or compound refers to that amount of the compound sufficient to result in amelioration of one or more symptoms of the disease being treated. When applied to an individual active ingredient, administered alone, a therapeutically effective dose refers to that ingredient alone. When applied to a combination, a therapeutically effective dose refers to combined amounts of the active ingredients that result in the therapeutic effect, whether administered serially or simultaneously. The invention specifically contemplates that one or more specific binding molecules may be administered according to methods of the invention, each in an effective dose.

“An individual having, or suspected of having, a disease associated with aberrant B-cell activity” is an individual in whom a disease or a symptom of a disorder may be caused by aberrant B-cell activity or B-cell proliferation, may be exacerbated by aberrant B-cell activity, or may be relieved by regulation of B-cell activity. Examples of such diseases are a B-cell malignancy or B-cell cancer (for example, B-cell lymphoma, a B-cell leukemia or a B-cell myeloma), a disease characterized by autoantibody production or a disease characterized by inappropriate T-cell stimulation caused by inappropriate B-cell antigen presentation to T-cells or caused by other pathways involving B-cells.

In one exemplary aspect, an individual treated by methods of the invention demonstrates a response to treatment that is better than, or improved relative to, the response to treatment with rituximab. A response which is improved over treatment with rituximab refers to a clinical response wherein treatment by a method of the invention results in a clinical response in a patient that is better than a clinical response in a patient receiving rituximab therapy, such as rituximab. An improved response is assessed by comparison of clinical criteria well-known in the art and described herein. Exemplary criteria include, but are not limited to, duration of B cell depletion, reduction in B cell numbers overall, reduction in B cell numbers in a biological sample, reduction in tumor size, reduction in the number of tumors existing and/or appearing after treatment, and improved overall response as assessed by patients themselves and physicians, e.g., using an International Prognostic Index. The improvement may be in one or more than one of the clinical criteria. An improved response with the method of the invention may be due to an inadequate response to previous or current treatment with rituximab, for example, because of toxicity and/or inadequate efficacy of the rituximab treatment.

B-cell malignancies or B-cell cancers include B-cell lymphomas [such as various forms of Hodgkin's disease, non-Hodgkins lymphoma (NHL) or central nervous system lymphomas], leukemias [such as acute lymphoblastic leukemia (ALL), chronic lymphocytic leukemia (CLL), Hairy cell leukemia and chronic myoblastic leukemia] and myelomas (such as multiple myeloma). Additional B cell cancers include small lymphocytic lymphoma, B-cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, intravascular large B-cell lymphoma, primary effusion lymphoma, Burkitt lymphoma/leukemia, B-cell proliferations of uncertain malignant potential, lymphomatoid granulomatosis, and post-transplant lymphoproliferative disorder.

Burkitt's lymphoma (or “Burkitt's B cell malignancy”, or “Burkitt's tumor”, or “Malignant lymphoma, Burkitt's type”) is a cancer of the lymphatic system (in particular, B lymphocytes). It is named after Denis Parsons Burkitt, a surgeon who first described the disease in 1956 while working in equatorial Africa, and it is associated with c-myc gene translocation. One aspect of the invention includes exemplary responses with CD37-directed therapies, including SMIP-016 and TRU-016, to a non-Burkitt's B cell malignancy.

Non-Burkitt's B cell malignancies include, but are not limited to, B-cell chronic lymphocytic leukemia (CLL)/small lymphocytic lymphoma, B-cell prolymphocytic leukemia, an acute lymphoblastic leukemia (ALL), lymphoplasmacytic lymphoma (including, but not limited to, Waldenstrom's macroglobulinemia), marginal zone lymphomas (including, but not limited to, splenic marginal zone B-cell lymphoma, nodal marginal zone lymphoma, and extranodal marginal zone B-cell lymphoma of mucosa-associated lymphoid tissue (MALT) type), hairy cell leukemia, plasma cell myeloma/plasmacytoma, follicular lymphoma, mantle cell lymphoma (MCL), diffuse large cell B-cell lymphoma, transforming large B cell lymphoma, mediastinal large B-cell lymphoma, intravascular large B-cell lymphoma, primary effusion lymphoma, and non-Burkitt's non-Hodgkins lymphoma (NHL).

Burkitt's lymphoma can be divided into three main clinical variants: the endemic, the sporadic and the immunodeficiency-associated variants.

The endemic variant occurs in equatorial Africa. It is the most common malignancy of children in this area. Children affected with the disease often also had chronic malaria which is believed to have reduced resistance to the Epstein-Barr virus and allowed it to take hold. The endemic variant characteristically involves the jaw or other facial bone, distal ileum, cecum, ovaries, kidney or the breast.

The sporadic variant of Burkitt's lymphoma (also known as “non-African”) is another form of non-Hodgkin lymphoma found outside of Africa. The tumor cells have a similar appearance to the cancer cells of classical African or endemic Burkitt lymphoma. Again it is believed that impaired immunity provides an opening for development of the Epstein-Barr virus. Non-Hodgkins, which includes Burkitt's, accounts for 30-50% of childhood lymphoma. The jaw is less commonly involved in the sporadic variant as compared to the endemic variant. The ileo-cecal region is the common site of involvement in the sporadic variant.

Immunodeficiency-associated Burkitt's lymphoma is usually associated with HIV infection or occurs in the setting of post-transplant patients who are taking immunosuppressive drugs. Actually, Burkitt's lymphoma can be one of the initial manifestations of AIDS.

By morphology (i.e. microscopic appearance) or immunophenotype, it is almost impossible to differentiate these three clinical variants. Immunodeficiency-associated Burkitt lymphoma may demonstrate more plasmacytic appearance or more pleomorphism, but these features are not specific.

Disorders characterized by autoantibody production are often considered autoimmune diseases. Autoimmune diseases include, but are not limited to: arthritis, rheumatoid arthritis, juvenile rheumatoid arthritis, osteoarthritis, polychondritis, psoriatic arthritis, psoriasis, dermatitis, polymyositis/dermatomyositis, inclusion body myositis, inflammatory myositis, toxic epidermal necrolysis, systemic scleroderma and sclerosis, CREST syndrome, responses associated with inflammatory bowel disease, Crohn's disease, ulcerative colitis, respiratory distress syndrome, adult respiratory distress syndrome (ARDS), meningitis, encephalitis, uveitis, colitis, glomerulonephritis, allergic conditions, eczema, asthma, conditions involving infiltration of T cells and chronic inflammatory responses, atherosclerosis, autoimmune myocarditis, leukocyte adhesion deficiency, systemic lupus erythematosus (SLE), subacute cutaneous lupus erythematosus, discoid lupus, lupus myelitis, lupus cerebritis, juvenile onset diabetes, multiple sclerosis, allergic encephalomyelitis, neuromyelitis optica, rheumatic fever, Sydenham's chorea, immune responses associated with acute and delayed hypersensitivity mediated by cytokines and T-lymphocytes, tuberculosis, sarcoidosis, granulomatosis including Wegener's granulomatosis and Churg-Strauss disease, agranulocytosis, vasculitis (including hypersensitivity vasculitis/angiitis, ANCA and rheumatoid vasculitis), aplastic anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, central nervous system (CNS) inflammatory disorders, multiple organ injury syndrome, myasthenia gravis, antigen-antibody complex mediated diseases, anti-glomerular basement membrane disease, anti-phospholipid antibody syndrome, allergic neuritis, Behcet disease, Castleman's syndrome, Goodpasture's syndrome, Lambert-Eaton Myasthenic Syndrome, Reynaud's syndrome, Sjorgen's syndrome, Stevens-Johnson syndrome, solid organ transplant rejection, graft versus host disease (GVHD), pemphigoid bullous, pemphigus, autoimmune polyendocrinopathies, seronegative spondyloarthropathies, Reiter's disease, stiff-man syndrome, giant cell arteritis, immune complex nephritis, IgA nephropathy, IgM polyneuropathies or IgM mediated neuropathy, idiopathic thrombocytopenic purpura (ITP), thrombotic thrombocytopenic purpura (TTP), Henoch-Schonlein purpura, autoimmune thrombocytopenia, autoimmune disease of the testis and ovary including autoimmune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes also referred to as insulin-dependent diabetes mellitus (IDDM) and Sheehan's syndrome; autoimmune hepatitis, lymphoid interstitial pneumonitis (HIV), bronchiolitis obliterans (non-transplant) vs NSIP, Guillain-Barre' Syndrome, large vessel vasculitis (including polymyalgia rheumatica and giant cell (Takayasu's) arteritis), medium vessel vasculitis (including Kawasaki's disease and polyarteritis nodosa), polyarteritis nodosa (PAN) ankylosing spondylitis, Berger's disease (IgA nephropathy), rapidly progressive glomerulonephritis, primary biliary cirrhosis, Celiac sprue (gluten enteropathy), cryoglobulinemia, cryoglobulinemia associated with hepatitis, amyotrophic lateral sclerosis (ALS), coronary artery disease, familial Mediterranean fever, microscopic polyangiitis, Cogan's syndrome, Whiskott-Aldrich syndrome and thromboangiitis obliterans.

Rheumatoid arthritis (RA) is a chronic disease characterized by inflammation of the joints, leading to swelling, pain, and loss of function. Patients having RA for an extended period usually exhibit progressive joint destruction, deformity, disability and even premature death.

Crohn's disease and a related disease, ulcerative colitis, are the two main disease categories that belong to a group of illnesses called inflammatory bowel disease (IBD). Crohn's disease is a chronic disorder that causes inflammation of the digestive or gastrointestinal (GI) tract. Although it can involve any area of the GI tract from the mouth to the anus, it most commonly affects the small intestine and/or colon. In ulcerative colitis, the GI involvement is limited to the colon.

Crohn's disease may be characterized by antibodies against neutrophil antigens, i.e., the “perinuclear anti-neutrophil antibody” (pANCA), and Saccharomyces cervisiae, i.e. the “anti-Saccharomyces cervisiae antibody” (ASCA). Many patients with ulcerative colitis have the pANCA antibody in their blood, but not the ASCA antibody, while many Crohn's patients exhibit ASCA antibodies, and not pANCA antibodies. One method of evaluating Crohn's disease is using the Crohn's disease Activity Index (CDAI), based on 18 predictor variables scores collected by physicians. CDAI values of 150 and below are associated with quiescent disease; values above that indicate active disease, and values above 450 are seen with extremely severe disease [Best et al., “Development of a Crohn's disease activity index.” Gastroenterology 70:439-444 (1976)]. However, since the original study, some researchers use a ‘subjective value’ of 200 to 250 as an healthy score. Systemic Lupus Erythematosus (SLE) is an autoimmune disease caused by recurrent injuries to blood vessels in multiple organs, including the kidney, skin, and joints. In patients with SLE, a faulty interaction between T cells and B-cells results in the production of autoantibodies that attack the cell nucleus. There is general agreement that autoantibodies are responsible for SLE, so new therapies that deplete the B-cell lineage, allowing the immune system to reset as new B-cells are generated from precursors, would offer hope for long lasting benefit in SLE patients.

Multiple sclerosis (MS) is also an autoimmune disease. It is characterized by inflammation of the central nervous system and destruction of myelin, which insulates nerve cell fibers in the brain, spinal cord, and body. Although the cause of MS is unknown, it is widely believed that autoimmune T cells are primary contributors to the pathogenesis of the disease. However, high levels of antibodies are present in the cerebral spinal fluid of patients with MS, and some theories predict that the B-cell response leading to antibody production is important for mediating the disease.

Autoimmune thyroid disease results from the production of autoantibodies that either stimulate the thyroid to cause hyperthyroidism (Graves' disease) or destroy the thyroid to cause hypothyroidism (Hashimoto's thyroiditis). Stimulation of the thyroid is caused by autoantibodies that bind and activate the thyroid stimulating hormone (TSH) receptor. Destruction of the thyroid is caused by autoantibodies that react with other thyroid antigens. Sjogren's syndrome is an autoimmune disease characterized by destruction of the body's moisture-producing glands.

Immune thrombocytopenic purpura (ITP) is caused by autoantibodies that bind to blood platelets and cause their destruction. Myasthenia Gravis (MG) is a chronic autoimmune neuromuscular disorder characterized by autoantibodies that bind to acetylcholine receptors expressed at neuromuscular junctions leading to weakness of the voluntary muscle groups.

Psoriasis, is characterized by autoimmune inflammation in the skin and also associated with arthritis in 30% of cases, scleroderma, inflammatory bowel disease, including Crohn's disease and ulcerative colitis, Also contemplated is the treatment of idiopathic inflammatory myopathy (IIM), including dermatomyositis (DM) and polymyositis (PM). Inflammatory myopathies have been categorized using a number of classification schemes. Miller's classification schema (Miller, Rheum Dis Clin North Am. 20:811-826, 1994) identifies 2 idiopathic inflammatory myopathies (IIM), polymyositis (PM) and dermatomyositis (DM).

Polymyositis and dermatomyositis are chronic, debilitating inflammatory diseases that involve muscle and, in the case of DM, skin. These disorders are rare, with a reported annual incidence of approximately 5 to 10 cases per million adults and 0.6 to 3.2 cases per million children per year in the United States (Targoff, Curr Probl Dermatol. 1991, 3:131-180). Idiopathic inflammatory myopathy is associated with significant morbidity and mortality, with up to half of affected adults noted to have suffered significant impairment (Gottdiener et al., Am J. Cardiol. 1978, 41:1141-49). Miller (Rheum Dis Clin North Am. 1994, 20:811-826 and Arthritis and Allied Conditions, Ch. 75, Eds. Koopman and Moreland, Lippincott Williams and Wilkins, 2005) sets out five groups of criteria used to diagnose IIM, i.e., Idiopathic Inflammatory Myopathy Criteria (IIMC) assessment, including muscle weakness, muscle biopsy evidence of degeneration, elevation of serum levels of muscle-associated enzymes, electromagnetic triad of myopathy, evidence of rashes in dermatomyositis, and also includes evidence of autoantibodies as a secondary criteria.

IIM associated factors, including muscle-associated enzymes and autoantibodies include, but are not limited to, creatine kinase (CK), lactate dehydrogenase, aldolase, C-reactive protein, aspartate aminotransferase (AST), alanine aminotransferase (ALT), and antinuclear autoantibody (ANA), myositis-specific antibodies (MSA), and antibody to extractable nuclear antigens.

A “binding molecule” according to the invention can be, for example, a protein (a “protein” may be polypeptide or peptide), nucleic acid, carbohydrate, lipid, or small molecule compound that binds to a target. A type of proteinaceous binding molecule contemplated by the invention is an antibody or an antibody fragment that retains binding activity. A binding molecule may be modified according to methods standard in the art to improve its binding affinity, diminish its immunogenicity, alter its effector functions and/or improve its availability in the body of an individual. Such modifications may include, for example, amino acid sequence modifications or expression as a fusion protein. Such fusion proteins are also binding molecules according to the invention. An exemplary binding molecule of the invention is a small modular immunopharmaceutical (SMIP™).

A binding molecule that is “specific” for a target binds to that target with a greater affinity than any other target. For example, a CD37-specific binding molecule binds to CD37 with a greater affinity than to any other target and a CD20-specific binding molecule binds to CD20 with a greater affinity than to any other target. Binding molecules of the invention may have affinities for their targets of a Ka of greater than or equal to about 10⁴ M⁻¹, preferably of greater than or equal to about 10⁵ M⁻¹, more preferably of greater than or equal to about 10⁶ M⁻¹ and still more preferably of greater than or equal to about 10⁷ M⁻¹. Affinities of even greater than about 10⁷ M⁻¹ are still more preferred, such as affinities equal to or greater than about 10⁷ M⁻¹, about 10⁸ M⁻¹, and about 10⁹ M⁻¹, and about 10¹⁰ M⁻¹. Affinities of binding molecules according to the present invention can be readily determined using conventional techniques, for example those described by Scatchard et al., Ann. N.Y. Acad. Sci. 51:660 (1949).

Certain CD37-specific binding molecules contemplated by the invention have affinities for CD37 of about 0.5 to about 10 nM. Certain CD20-specific binding molecules contemplated by the invention have affinities for CD20 of about 1 to about 30 nM.

Another characteristic of certain CD37-binding molecules and CD20-binding molecules contemplated by the invention is they exhibit a half life in circulation of about 7 to about 30 days.

CD37-specific antibodies that characterized the CD37 antigen in the Thrid HLDA Workshop were HD28, G28-1, HH1, BI14, WR17 and F93G6. See, Ling and MacLennan, pp. 302-335 in Leucocyte Typing III. White Cell Differentiation Antigens, Oxford University Press (1987). Other CD37-specific antibodies that have been described include RFB-7, Y29/55, MB-1, M-B371, M-B372 and IPO-24. See, Moldenhaurer, J. Biol., Regul. Homeost. Agents, 14: 281-283 (2000) which states that all these antibodies recognize only one CD37 epitope. Schwartz-Albiez et al., 14: 905-914 (1988) indicates that the epitope is situated in the carbohydrate moiety of CD37. Another CD37-specific antibody is S-B3 (Biosys).

Patents and patent publications describing CD20 antibodies include U.S. Pat. Nos. 5,776,456, 5,736,137, 6,399,061, and 5,843,439, as well as US patent application Nos. US 2002/0197255A1 and US 2003/0021781A1 (Anderson et al.); U.S. Pat. No. 6,455,043B1 and WO00/09160 (Grillo-Lopez, A.); WO00/27428 (Grillo-Lopez and White); WO00/27433 (Grillo-Lopez and Leonard); WO00/44788 (Braslawsky et al.); WO01/10462 (Rastetter, W.); WO01/10461 (Rastetter and White); WO01/10460 (White and Grillo-Lopez); US appln No. US2002/0006404 and WO02/04021 (Hanna and Hariharan); US appln No. US2002/0012665 A1 and WO01/74388 (Hanna, N.); US appln No. US2002/0009444A1, and WO01/80884 (Grillo-Lopez, A.); WO01/97858 (White, C.); US appln No. US2002/0128488A1 and WO02/34790 (Reff, M.); WO02/060955 (Braslawsky et al.); WO02/096948 (Braslawsky et al.); WO02/079255 (Reff and Davies); U.S. Pat. No. 6,171,586B1, and WO98/56418 (Lam et al.); WO98/58964 (Raju, S.); WO99/22764 (Raju, S.); WO99/51642, U.S. Pat. No. 6,194,551B1, U.S. Pat. No. 6,242,195B1, U.S. Pat. No. 6,528,624B1 and U.S. Pat. No. 6,538,124 (Idusogie et al.); WO00/42072 (Presta, L.); WO00/67796 (Curd et al.); WO01/03734 (Grillo-Lopez et al.); US appln No. US 2002/0004587A1 and WO01/77342 (Miller and Presta); US appln No. US2002/0197256 (Grewal, I.); U.S. Pat. Nos. 6,090,365B1, 6,287,537B1, 6,015,542, 5,843,398, and 5,595,721, (Kaminski et al.); U.S. Pat. Nos. 5,500,362, 5,677,180, 5,721,108, and 6,120,767 (Robinson et al.); U.S. Pat. No. 6,410,391B1 (Raubitschek et al.); U.S. Pat. No. 6,224,866B1 and WO00/20864 (Barbera-Guillem, E.); WO01/13945 (Barbera-Guillem, E.); WO00/67795 (Goldenberg); WO00/74718 (Goldenberg and Hansen); WO00/76542 (Golay et al.); WO01/72333 (Wolin and Rosenblatt); U.S. Pat. No. 6,368,596B1 (Ghetie et al.); US Appln No. US2002/0041847A1, (Goldenberg, D.); US Appln no. US2003/0026801A1 (Weiner and Hartmann); WO02/102312 (Engleman, E.), each of which is expressly incorporated herein by reference. See, also, U.S. Pat. No. 5,849,898 and EP appln No. 330,191 (Seed et al.); U.S. Pat. No. 4,861,579 and EP332,865A2 (Meyer and Weiss); and WO95/03770 (Bhat et al.).

Rituximab has been approved for human clinical use as Rituxan®. Rituxan® is considered to be a CD20-specific binding molecule of the invention.

Small, modular immunopharmaceuticals (SMIPs) are considered to be one type of binding molecules of the invention. Methods for making SMIPs have been described previously in co-owned U.S. application Ser. No. 10/627,556 and US Patent Publ. 20030133939, 20030118592, and 20050136049, which are incorporated herein by reference in their entirety. SMIPs are novel binding domain-immunoglobulin fusion proteins that generally feature a binding domain for a cognate structure such as an antigen, a counterreceptor or the like, an IgG1, IGA or IgE hinge region polypeptide or a mutant IgG1 hinge region polypeptide having either zero, one or two cysteine residues, and immunoglobulin CH2 and CH3 domains. In one embodiment, the binding domain molecule has one or two cysteine (Cys) residues in the hinge region. In a related embodiment, when the binding domain molecule comprises two Cys residues, the first Cys, which is involved in binding between the heavy chain and light chain, is not deleted or substituted with an amino acid.

The binding domain of molecules useful in methods of the invention are contemplated as having one or more binding regions, such as variable light chain and variable heavy chain binding regions derived from one or more immunoglobulin superfamily members, such as an immunoglobulin. These regions, moreover, are typically separated by linker peptides, which may be any linker peptide known in the art to be compatible with domain or region joinder in a binding molecule. Exemplary linkers are linkers based on the Gly₄Ser linker motif, such as (Gly₄Ser)ₙ, where n=1-5. The molecules for use in the methods of the invention also contain sufficient amino acid sequence derived from a constant region of an immunoglobulin to provide an effector function, preferably ADCC and/or CDC. Thus, the molecules will have a sequence derived from a CH2 domain of an immunoglobulin or CH2 and CH3 domains derived from one or more immunoglobulins. SMIPs are capable of ADCC and/or CDC but are compromised in their ability to form disulfide-linked multimers.

The invention includes humanized CD37-specific SMIP polypeptides that exhibit at least 80 percent identity to the polypeptide set forth in SEQ ID NO: 2, wherein the humanized CD37-specific SMIP polypeptide binds CD37. In one aspect, the humanized CD37-specific SMIP polypeptides comprise any amino acid sequence selected from the group consisting of SEQ ID NOS: 6, 8, 10, 12, 14, 16, 18, 20, 22, 24, 26, 28, 30, 32, 34, 36, 38, 40, 42, 44, 46, 48, 50, 52, 54, 56, 58, 60, 80, 82, 84, 86, 88, and 222. In another aspect, the humanized CD37-specific SMIP polypeptides comprise at least one amino acid modification in a complementarity-determining region (CDR) selected from the group consisting of: light chain CDR1, heavy chain CDR1, light chain CDR2, heavy chain CDR2, light chain CDR3, and heavy chain CDR3.

In one embodiment, the invention includes a humanized CD37-specific SMIP polypeptide, wherein CDR1 of the light chain comprises the amino acid sequence of SEQ ID NO: 61 (RASENVYSYLA). The invention also includes a humanized CD37-specific SMIP polypeptide, wherein CDR1 of the light chain comprises the amino acid sequence of SEQ ID NO: 62 (RTSENVYSYLA). The invention further includes a humanized CD37-specific SMIP polypeptide, wherein CDR1 of the heavy chain comprises the amino acid sequence of SEQ ID NO: 63 (GYMNM).

In another embodiment, the invention includes a humanized CD37-specific SMIP polypeptide, wherein CDR2 of the light chain comprises the amino acid sequence of SEQ ID NO: 64 (FAKTLAE). The invention also includes a humanized CD37-specific SMIP polypeptide, wherein CDR2 of the heavy chain comprises the amino acid sequence of SEQ ID NO: 65 (NIDPYYGGTTTYNRKFKG).

In a further embodiment, the invention includes a humanized CD37-specific SMIP polypeptide, wherein CDR3 of the light chain comprises the amino acid sequence of SEQ ID NO: 66 (QHHSDNPWT). The invention further includes a humanized CD37-specific SMIP polypeptide, wherein CDR3 of the heavy chain comprises the amino acid sequence of SEQ ID NO: 67 (SVGPFDY). The invention further includes a humanized CD37-specific SMIP polypeptide, wherein CDR3 of the heavy chain comprises the amino acid sequence of SEQ ID NO: 68 (SVGPFDS). The invention also includes a humanized CD37-specific SMIP polypeptide, wherein CDR3 of the heavy chain comprises the amino acid sequence of SEQ ID NO: 69 (SVGPMDY).

In another aspect, the invention includes a humanized CD37-specific SMIP polypeptide comprising at least one, at least two, or at least three sequence(s) of the light chain CDR amino acid sequences selected from the group consisting of SEQ ID NOS: 61, 62, 64, and 66. In yet another embodiment, the invention includes a humanized CD37-specific SMIP polypeptide comprising a light chain CDR1 amino acid sequence of SEQ ID NOS: 61 or 62, or a variant thereof in which one or two amino acids of SEQ ID NOS: 61 or 62 has been changed; a light chain CDR2 amino acid sequence of SEQ ID NO: 64, or a variant thereof in which one or two amino acids of SEQ ID NO: 64 has been changed; and a light chain CDR3 amino acid sequence of SEQ ID NO: 66, or a variant thereof in which one or two amino acids of SEQ ID NO: 66 has been changed.

In still another aspect, the invention includes a humanized CD37-specific SMIP polypeptide comprising at least one, at least two, or at least three of the heavy chain CDR amino acid sequences selected from the group consisting of SEQ ID NOS: 63, 65, and 67-69. In a further embodiment, the invention includes a humanized CD37-specific SMIP polypeptide comprising a heavy chain CDR1 amino acid sequence of SEQ ID NO: 63, or a variant thereof in which one or two amino acids of SEQ ID NO: 63 has been changed; a heavy chain CDR2 amino acid sequence of SEQ ID NO: 65, or a variant thereof in which one or two amino acids of SEQ ID NO: 65 has been changed; and a heavy chain CDR3 amino acid sequence selected from the group consisting of SEQ ID NOS: 67-69, or a variant thereof in which one or two amino acids of any one of SEQ ID NOS: 67-69 has been changed.

The invention also includes humanized CD37-specific SMIP polypeptides comprising at least one amino acid modification in a framework region (FR) selected from the group consisting of: light chain FR1, heavy chain FR1, light chain FR2, heavy chain FR2, light chain FR3, heavy chain FR3, light chain FR4, and heavy chain FR4. In one embodiment, the invention includes a humanized CD37-specific SMIP polypeptide, wherein the first framework region (FR1) of the light chain comprises the amino acid sequence of SEQ ID NO: 70 (EIVLTQSPATLSLSPGERATLSC). In another embodiment, the invention includes a humanized CD37-specific SMIP polypeptide, wherein FR1 of the heavy chain comprises the amino acid sequence of SEQ ID NO: 71 (EVQLVQSGAEVKKPGESLKISCKGSGYSFT). In still another embodiment, the invention includes a humanized CD37-specific SMIP polypeptide, wherein FR2 of the light chain comprises the amino acid sequence of SEQ ID NO: 72 (WYQQKPGQAPRLLIY). In a further embodiment, the invention includes a humanized CD37-specific SMIP polypeptide, wherein FR2 of the heavy chain comprises the amino acid sequence of SEQ ID NO: 73 (WVRQMPGKGLEWMG). In yet another embodiment, the invention includes a humanized CD37-specific SMIP polypeptide, wherein FR3 of the light chain comprises the amino acid sequence of SEQ ID NO: 74 (GIPARFSGSGSGTDFTLTISSLEPEDFAVYYC). In yet another embodiment, the invention includes a humanized CD37-specific SMIP polypeptide, wherein FR3 of the heavy chain comprises the amino acid sequence of SEQ ID NO: 75 (QVTISADKSISTAYLQWSSLKASDTAMYYCAR). In yet another embodiment, the invention includes a humanized CD37-specific SMIP polypeptide, wherein FR4 of the light chain comprises the amino acid sequence of SEQ ID NO: 76 (FGQGTKVEIK). In yet another embodiment, the invention includes a humanized CD37-specific SMIP polypeptide, wherein FR4 of the heavy chain comprises the amino acid sequence of SEQ ID NO: 77 (WGQGTLVTVSS). In yet another embodiment, the invention includes a humanized CD37-specific SMIP polypeptide, wherein FR4 of the heavy chain comprises the amino acid sequence of SEQ ID NO: 78 (WGRGTLVTVSS).

The invention further includes humanized CD37-specific SMIP polypeptides comprising at least one, at least two, or at least three sequence(s) of the light chain FR amino acid sequences selected from the group consisting of SEQ ID NOS: 70, 72, 74, and 76. In one embodiment, the invention includes a humanized CD37-specific SMIP polypeptide comprising a light chain FR1 amino acid sequence of SEQ ID NO: 70, or a variant thereof in which one or two amino acids of SEQ ID NO: 70 has been changed; a light chain FR2 amino acid sequence of SEQ ID NO: 72, or a variant thereof in which one or two amino acids of SEQ ID NO: 72 has been changed; a light chain FR3 amino acid sequence of SEQ ID NO: 74, or a variant thereof in which one or two amino acids of SEQ ID NO: 74 has been changed; and a light chain FR4 amino acid sequence of SEQ ID NO: 76, or a variant thereof in which one or two amino acids of SEQ ID NO: 76 has been changed.

In addition, the invention includes humanized CD37-specific SMIP polypeptides comprising at least one, at least two, or at least three sequence(s) of the heavy chain FR amino acid sequences selected from the group consisting of SEQ ID NOS: 71, 73, 75, 77, and 78. In one embodiment, the invention includes a humanized CD37-specific SMIP polypeptide comprising a heavy chain FR1 amino acid sequence of SEQ ID NO: 71, or a variant thereof in which one or two amino acids of SEQ ID NO: 71 has been changed; a heavy chain FR2 amino acid sequence of SEQ ID NO: 73, or a variant thereof in which one or two amino acids of SEQ ID NO: 73 has been changed; a heavy chain FR3 amino acid sequence of SEQ ID NO: 75, or a variant thereof in which one or two amino acids of SEQ ID NO: 75 has been changed; and a heavy chain FR4 amino acid sequence of SEQ ID NOS: 77 or 78, or a variant thereof in which one or two amino acids of SEQ ID NOS: 77 or 78 has been changed.

The invention also includes an isolated nucleic acid molecule comprising a nucleotide sequence encoding a humanized CD37-specific SMIP polypeptide that exhibits at least 80 percent identity to the polypeptide set forth in SEQ ID NO: 2, wherein the humanized CD37-specific SMIP polypeptide binds CD37. Such an isolated nucleic acid molecule may comprise a nucleotide sequence selected from the group consisting of: SEQ ID NOS: 5, 7, 9, 11, 13, 15, 17, 19, 21, 23, 25, 27, 29, 31, 33, 35, 37, 39, 41, 43, 45, 47, 49, 51, 53, 55, 57, 59, 79, 81, 83, 85, 87, and 221. In one embodiment, the invention includes vectors that comprise these nucleic acid molecules and host cells that comprise the vectors.

The invention also includes processes of producing the polypeptides described herein, comprising culturing the host cells under suitable conditions to express the polypeptides, and optionally isolating the polypeptides from the culture.

In yet another aspect, the invention includes compositions comprising the humanized CD37-specific SMIP polypeptides of the invention and a pharmaceutically acceptable carrier.

The invention further includes using the CD37-specific SMIP or CD37-specific binding molecules described herein in any of the methods of the invention. Such methods include the use of any of the CD37-specific SMIP or CD37-specific binding molecule comprising an amino acid sequence selected from the group consisting of SEQ ID NOS: 6, 8, 10, 12, 14, 16, 18, 20, 22, 24, 26, 28, 30, 32, 34, 36, 38, 40, 42, 44, 46, 48, 50, 52, 54, 56, 58, 60, 80, 82, 84, 86, 88, and 222.

In some embodiments of the invention, the CD37-specific binding molecules or CD37-specific SMIPs are not radiolabeled.

In yet another aspect, the invention includes kits for reducing B-cells comprising the compositions of the invention; and protocols for using the kits to reduce B cells. Such kits may further comprise one or more CD20-specific binding molecule(s). The invention contemplates that such a CD20-specific binding molecule is TRU-015.

The invention also includes humanized CD37-specific SMIP polypeptides comprising a CDR1, a CDR2, and a CDR3, that exhibits at least 80 percent identity to the polypeptide set forth in SEQ ID NO: 2. Such CD37-specific SMIP polypeptides may further comprise a human framework domain separating each of CDR1, CDR2, and CDR3.

In another aspect, the invention includes a humanized CD37-specific SMIP polypeptide that exhibits at least 80 percent identity to the polypeptide set forth in SEQ ID NO: 2, wherein the humanized CD37-specific SMIP polypeptide binds CD37 and comprises a hinge region polypeptide comprising an amino acid sequence selected from the group consisting of SEQ ID NOS: 90, 92, 94, 96, 98, 100, 102, 104, 106, 108 110, 112, 114, 115, 116, 118, 120, 122, 124, 126 and 127.

The invention also contemplates a humanized CD37-specific SMIP polypeptide that exhibits at least 80 percent identity to the polypeptide set forth in SEQ ID NO: 2, wherein the humanized CD37-specific SMIP polypeptide binds CD37 and comprises a linker comprising (Gly4Ser)n, wherein n is 1, 2, 3, 4, 5, or 6.

In still a further aspect, the invention includes a humanized CD37-specific SMIP polypeptide, wherein CDR1 of the light chain comprises the amino acid sequence selected from the group consisting of SEQ ID NOS: 128 (RTSQNVYSYLA), 129 (RTSESVYSYLA), 130 (RASQSVYSYLA), 131 (RASQSVSSYLA) and 132 (RASQSVSYYLA). In another embodiment, the invention includes a humanized CD37-specific SMIP polypeptide, wherein CDR1 of the heavy chain comprises the amino acid sequence selected from the group consisting of SEQ ID NOS: 133 (SYMNM) and 134 (SYWIG). In a further embodiment, the invention includes a humanized CD37-specific SMIP polypeptide, wherein CDR2 of the light chain comprises the amino acid sequence selected from the group consisting of SEQ ID NOS: 135 (AASSLQS), 136 (GASTRAT) and 137 (DASNRAT). In still another embodiment, the invention includes a humanized CD37-specific SMIP polypeptide, wherein CDR2 of the heavy chain comprises the amino acid sequence selected from the group consisting of SEQ ID NOS: 138 (IIYPGDSDTRYSPSFQG) and 139 (RIDPSDSYTNYSPSFQG).

The invention also includes a humanized CD37-specific SMIP polypeptide, wherein CDR3 of the light chain comprises the amino acid sequence of SEQ ID NO: 220 (QHHSDNPWT). In another embodiment, the invention includes a humanized CD37-specific SMIP polypeptide, wherein CDR3 of the heavy chain comprises the amino acid sequence selected from the group consisting of SEQ ID NOS: 211 (SVGPMDY), 212 (SVGPFDY), 213 (SVGPMDV), 214 (SVGPFDS), 215 (SVGPFDP), 216 (SVGPFQH), 217 (SVGPFDV), 218 (SVGPFDI) and 219 (SVGPFDL).

In still a further aspect, the invention includes CD37-specific SMIP polypeptides with alternative framework regions. In one aspect, the invention includes a humanized CD37-specific SMIP polypeptide, wherein FR1 of the light chain comprises the amino acid sequence selected from the group consisting of SEQ ID NOS: 170-181. In another aspect, the invention includes a humanized CD37-specific SMIP polypeptide, wherein FR1 of the heavy chain comprises the amino acid sequence selected from the group consisting of SEQ ID NOS: 140-146. In a still further aspect, the invention includes a humanized CD37-specific SMIP polypeptide, wherein FR2 of the light chain comprises the amino acid sequence selected from the group consisting of SEQ ID NOS: 182-193. In yet another aspect, the invention includes a humanized CD37-specific SMIP polypeptide, wherein FR2 of the heavy chain comprises the amino acid sequence selected from the group consisting of SEQ ID NOS: 147-153. In an additional aspect, the invention includes a humanized CD37-specific SMIP polypeptide, wherein FR3 of the light chain comprises the amino acid sequence selected from the group consisting of SEQ ID NOS: 194-205. In yet another aspect, the invention includes a humanized CD37-specific SMIP polypeptide, wherein FR3 of the heavy chain comprises the amino acid sequence selected from the group consisting of SEQ ID NOS: 154-160. In a further aspect, the invention includes a humanized CD37-specific SMIP polypeptide, wherein FR4 of the light chain comprises the amino acid sequence selected from the group consisting of SEQ ID NOS: 206-210. In yet another aspect, the invention includes a humanized CD37-specific SMIP polypeptide, wherein FR4 of the heavy chain comprises the amino acid sequence selected from the group consisting of SEQ ID NOS: 161-169.

Exemplary CD37-specific SMIPs useful in the invention include, but are not limited to: G28-1 scFv (SSS-S)H WCH2 WCH3, consists of a G28-1 single chain Fv in which all three cysteine residues in the connection or hinge regions are mutated to serine residues, and wild type CH2 and CH3 domains; G28-1 scFv IgAH WCH2 WCH3, comprising an IgA hinge and WT IgG1 domains; G28-1 scFv VHL11S (SSS-S)H WCH2CH3 in which all three cysteine residues in the connection or hinge regions are mutated to serine residues and the leucine at position 11 of the heavy chain variable region is substituted with a serine; G28-1 scFv VH L11S (CSS-S)H WCH2 CH3, in which cysteine residues were substituted at the second and third positions with serine; G28-1 scFv VHL11S (CSC-S)H WCH2 CH3, in which cysteine residues were substituted at the second position with serine; G28-1 scFv VH11S (SSC-P)H WCH2 WCH3 (referred to as TRU-016 herein), in which the first and second cysteine residues in the connection or hinge regions are mutated to serine residues and the leucine at position 11 of the heavy chain variable region is substituted with a serine; G28-1 scFv VH11 S(SCS-S)H WCH2 WCH3, in which the first and third cysteine residues in the hinge regions are mutated to serine residues; G28-1 scFv VHL11 S(CCS-P)H WCH2 WCH3, in which the third cysteine residue in the hinge region is substituted with a serine; G28-1 scFv VHL11S (SCC-P)H WCH2 WCH3, in which the first cysteine is substituted with a serine; G28-1 scFv VH L11S mIgE CH2 CH3 CH4, comprising mouse IgE CH 2-4 regions in which the leucine at position 11 of the heavy chain variable region is substituted with a serine; G28-1 scFv VH L11S mIgA WIgACH2 T4CH₃, comprising a mouse IgA hinge with a wild type IgA CH2 and a truncated IgA CH3 domain lacking the 4 carboxy amino acids GTCY; G28-1 scFv VHL11S hIgE CH2 CH3 CH4, comprising IgE CH regions in which the leucine at position 11 of the heavy chain variable region is substituted with a serine; and G28-1 scFv VHL11S hIgAH WIgACH2 TCH3, comprising an IgA hinge, a wild type IgA CH2 and a truncated IgA CH2 and a truncated IgA CH3 domain lacking the 4 carboxy amino acids GTCY.

Exemplary CD20-specific SMIPs useful in the invention include SMIPs derived from the anti-CD20 monoclonal antibody 2H7 described in US Patent Publ. 2003133939. and 20030118592. The SMIPs include 2H7scFv-1g or a derivative thereof. Derivatives includes CytoxB-MHWTG1C, which has a human IgG1 Fc domain and a mutant IgG1 hinge domain; CytoxB-MHMG1C, which comprises a mutated Fc domain; MG1H/MG1C, which comprises an Fc receptor with a mutated leucine residue 234; CytoxB-IgAHWTHG1C, comprising a portion of the human IgA hinge fused to wild-type human Fc domain; 2H7 scFv-llama IgG1, comprising the llama IgG1 hinge and CH2CH3 regions, 2H7 scFv-llama IgG2, comprising the llama IgG2 hinge and CH2CH3 regions; 2H7 scFv-llama IgG3, comprising the llama IgG3 hinge and CH2CH3 regions.

2H7 scFv MTH(SSS) WTCH2CH3, in which all three cysteine residues in the connection or hinge regions are mutated to serine residues, and wild type CH2 and CH3 domains; 2H7 scFv MTH (SSC), in which the first two cysteine residues were substituted with serine residues; 2H7 scFv MTH (SCS), in which the first and third cysteines were substituted with serine residues; 2H7 scFv MTH(CSS) WTCH2CH3, in which cysteine residues were substituted at the second and third positions with serine; 2H7 scFv VH11 SER IgG MTH(SSS) WTCH2CH3, in which the leucine at position 11 in the heavy chain variable region is substituted with serine; 2H7 scFv IgA hinge-IgG1 CH2-CH3, comprising an IgA hinge region and WT IgG1 domains; 2H7 scFv IgA hinge-CH2-CH3, comprising IgA hinge, CH2-3 regions; 2H7 IgAWH IgACH2-T4-CH3, comprising an IgA hinge, a wild type IgA CH2 and a truncated IgA CH3 domain lacking the 4 carboxy amino acids GTCY. Derivatives with mutations in the IgG CH3 region include 2H7 scFv MTH WTCH2 MTCH3 Y405, in which phenylalanine residue at position 405 (numbering according to Kabat et al. supra) was substituted with tyrosine; 2H7 scFv MTH WTCH2 MTCH3 A405, in which phenylalanine position at 405 was substituted with an alanine; scFv MTH WTCH2 MTCH3 A407, in which tyrosine residue at position 407 was substituted with an alanine; scFv MTH WTCH2 MTCH3 Y405A407, comprising the two mutations; and scFv MTH WTCH2 MTCH3 A405A407 comprising two mutations. 2H7 scFv MTH(CCS) WTCH2CH3 is a construct with the third cysteine residue in the IgG1 hinge region substituted with a serine residue. The 2H7 scFv IgG MTH(SSS) MTCH2WTCH3 SMIP comprises mutant hinge (MT (SSS)) and a mutant CH2 domain in which the proline at residue 238 (according to Ward et al.,) was substituted with a serine.

2H7scFv-Ig derivatives also include 2H7 scFv mutants with point mutations in the variable heavy chain region. The following constructs all comprise mutations in which the leucine at position 11 in the heavy chain variable region is substituted with serine: 2H7 scFv VH11SER IgG MTH (SSS-S) WTCH2CH3, 2H7scFv VHL11S (CSS-S)H WCH2 WCH3, comprising a mutated hinge region as set out above; 2H7scFv VHL11S (CSC-S)H WCH2 WCH3 comprising a mutated hinge region as set out above; 2H7 scFv VHL11S IgAH IgACH₂ T4CH3, comprises the IgA hinge, WT IgA CH2 and truncated IgA CH3; 2H7 scFv VHL11S IgECH2 CH3 CH4, comprising the IgE CH 2-4 regions; 2H7 VHL11S scFv (SSS-S) IgECH3CH4, comprising a mutated hinge region and IgE CH3 and CH4 regions; 2H7 scFv VH L11S mIgE CH2 CH3 CH4, comprises mouse IgE regions; 2H7 scFv VH L11S mIgAH WIGACH2 T4CH3 comprises the mutations described above and a mouse IgA constant region consisting of a wild type CH2 region and a mutated CH3 region; 2H7 scFv VH L11S (SSS-S)H K322S CH2 WCH3 comprises a mutation in the human IgG1 CH2 region at residue 322, where lysine was changed to serine; 2H7 scFv VH L11S(CSS-S)H K322S CH2 WCH3 comprises a mutated hinge region as described above, and a mutated CH2 region as previously described; 2H7 scFv VH L11S (SSS-S)H P331S CH2 WCH3, comprises a mutated hinge region as described above, and a mutated CH2 region in which proline at residue 331 was changed to a serine; 2H7 scFv VH L11S(CSS-S)H P331S CH2 WCH3 comprises a mutated hinge region and a proline to serine mutation at residue 331 in the CH2 region; 2H7 scFv VH L11S (SSS-S) H T256N CH2 WCH3, comprises a mutated hinge region and a threonine to asparagine mutation at residue 256 in the CH2 region; 2H7 scFv VH L11 S (SSS-S)H RTPE/QNAK (255-258) CH2 WCH3, comprises a mutated hinge region and a series of mutations in which residues 255-258 have been mutated from arginine, threonine, proline, glutamic acid to glutamine, asparagines, alanine and lysine, respectively; 2H7 scFv VH L11S (SSS-S)H K290Q CH2 WCH3, comprises a mutated hinge regions and a lysine to glutamine change at position 290; 2H7 scFv VH L11S (SSS-S)H A339P CH₂ WCH3, comprises a mutated hinge region and an alanine to proline change at position 339; SMIP 2H7 scFv (SSS-S)H P238SCH2 WCH3, comprises a mutated hinge region and an proline to serine change at position 238 in CH2, which is the same as 2H7 scFv IgG MTH (SSS) MTCH2WTCH3. 2H7 scFv IgAH IGAHCH2 T18CH3 comprises a wild type IgA hinge and CH2 region and a CH3 region with an18 amino acid truncation at the carboxy end.

A binding molecule of the invention may comprise a native or engineered extracellular domain from another protein which improves the binding molecule activity. In one embodiment, the extracellular domain is selected from the group consisting of CD154 and CTLA4.

A “synergistic combination” of CD37-specific binding molecules and CD20-specific binding molecules is a combination that has an effect that is greater than the sum of the effects of the binding molecules when administered alone.

In one aspect of the invention, the binding molecules are administered in one or more pharmaceutical compositions. To administer the binding molecules to human or test animals, it is preferable to formulate the binding molecules in a composition comprising one or more pharmaceutically acceptable carriers. The phrase “pharmaceutically or pharmacologically acceptable” refer to molecular entities and compositions that do not produce allergic, or other adverse reactions when administered using routes well-known in the art, as described below. “Pharmaceutically acceptable carriers” include any and all clinically useful solvents, dispersion media, coatings, antibacterial and antifungal agents, isotonic and absorption delaying agents and the like.

In addition, compounds may form solvates with water or common organic solvents. Such solvates are contemplated as well.

The binding molecule compositions may be administered orally, topically, transdermally, parenterally, by inhalation spray, vaginally, rectally, or by intracranial injection. The term parenteral as used herein includes subcutaneous injections, intravenous, intramuscular, intracisternal injection, or infusion techniques. Administration by intravenous, intradermal, intramusclar, intramammary, intraperitoneal, intrathecal, retrobulbar, intrapulmonary injection and or surgical implantation at a particular site is contemplated as well. Generally, compositions are essentially free of pyrogens, as well as other impurities that could be harmful to the recipient. Injection, especially intravenous, is preferred.

Pharmaceutical compositions of the present invention containing binding molecules used in a method of the invention may contain pharmaceutically acceptable carriers or additives depending on the route of administration. Examples of such carriers or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form of the present invention.

Formulation of the pharmaceutical composition will vary according to the route of administration selected (e.g., solution, emulsion). An appropriate composition comprising the antibody to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers

A variety of aqueous carriers, e.g., water, buffered water, 0.4% saline, 0.3% glycine, or aqueous suspensions may contain the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate.

The binding molecule compositions can be lyophilized for storage and reconstituted in a suitable carrier prior to use. This technique has been shown to be effective with conventional immunoglobulins. Any suitable lyophilization and reconstitution techniques can be employed. It will be appreciated by those skilled in the art that lyophilization and reconstitution can lead to varying degrees of antibody activity loss and that use levels may have to be adjusted to compensate.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above.

The concentration of binding molecule in these formulations can vary widely, for example from less than about 0.5%, usually at or at least about 1% to as much as 15 or 20% by weight and will be selected primarily based on fluid volumes, viscosities, etc., in accordance with the particular mode of administration selected. Thus, a typical pharmaceutical composition for parenteral injection could be made up to contain 1 mL sterile buffered water, and 50 mg of antibody. A typical composition for intravenous infusion could be made up to contain 250 mL of sterile Ringer's solution, and 150 mg of antibody. Actual methods for preparing parenterally administrable compositions will be known or apparent to those skilled in the art and are described in more detail in, for example, Remington's Pharmaceutical Science, 15th ed., Mack Publishing Company, Easton, Pa. (1980). An effective dosage of antibody is within the range of 0.01 mg to 1000 mg per kg of body weight per administration.

The pharmaceutical compositions may be in the form of a sterile injectable aqueous, oleaginous suspension, dispersions or sterile powders for the extemporaneous preparation of sterile injectable solutions or dispersions. The suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. The carrier can be a solvent or dispersion medium containing, for example, water, ethanol, polyol (for example, glycerol, propylene glycol, and liquid polyethylene glycol, and the like), suitable mixtures thereof, vegetable oils, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

In all cases the form must be sterile and must be fluid to the extent that easy syringability exists. The proper fluidity can be maintained, for example, by the use of a coating, such as lecithin, by the maintenance of the required particle size in the case of dispersion and by the use of surfactants. It must be stable under the conditions of manufacture and storage and must be preserved against the contaminating action of microorganisms, such as bacteria and fungi. The prevention of the action of microorganisms can be brought about by various antibacterial an antifungal agents, for example, parabens, chlorobutanol, phenol, sorbic acid, thimerosal, and the like. In many cases, it will be desirable to include isotonic agents, for example, sugars or sodium chloride. Prolonged absorption of the injectable compositions can be brought about by the use in the compositions of agents delaying absorption, for example, aluminum monostearate and gelatin.

Compositions useful for administration may be formulated with uptake or absorption enhancers to increase their efficacy. Such enhancers include for example, salicylate, glycocholate/linoleate, glycholate, aprotinin, bacitracin, SDS, caprate and the like. See, e.g., Fix (J. Pharm. Sci., 85:1282-1285, 1996) and Oliyai and Stella (Ann. Rev. Pharmacol. Toxicol., 32:521-544, 1993).

In addition, the properties of hydrophilicity and hydrophobicity of the compositions contemplated for use in the invention are well balanced, thereby enhancing their utility for both in vitro and especially in vivo uses, while other compositions lacking such balance are of substantially less utility. Specifically, compositions contemplated for use in the invention have an appropriate degree of solubility in aqueous media which permits absorption and bioavailability in the body, while also having a degree of solubility in lipids which permits the compounds to traverse the cell membrane to a putative site of action. Thus, antibody compositions contemplated are maximally effective when they can be delivered to the site of target antigen activity.

In one aspect, methods of the invention include a step of administration of a binding molecule composition.

Methods of the invention are performed using any medically-accepted means for introducing a therapeutic directly or indirectly into a mammalian individual, including but not limited to injections, oral ingestion, intranasal, topical, transdermal, parenteral, inhalation spray, vaginal, or rectal administration. The term parenteral as used herein includes subcutaneous, intravenous, intramuscular, and intracisternal injections, as well as catheter or infusion techniques. Administration by, intradermal, intramammary, intraperitoneal, intrathecal, retrobulbar, intrapulmonary injection and or surgical implantation at a particular site is contemplated as well.

In one embodiment, administration is performed at the site of a cancer or affected tissue needing treatment by direct injection into the site or via a sustained delivery or sustained release mechanism, which can deliver the formulation internally. For example, biodegradable microspheres or capsules or other biodegradable polymer configurations capable of sustained delivery of a composition (e.g., a soluble polypeptide, antibody, or small molecule) can be included in the formulations of the invention implanted near the cancer.

Therapeutic compositions may also be delivered to the patient at multiple sites. The multiple administrations may be rendered simultaneously or may be administered over a period of time. In certain cases it is beneficial to provide a continuous flow of the therapeutic composition. Additional therapy may be administered on a period basis, for example, hourly, daily, weekly or monthly.

Binding molecule compositions of the invention may comprise one, or may comprise more than one, binding molecules. Also contemplated by the present invention is the administration of binding molecule compositions in conjunction with a second agent. Second agents contemplated by the invention are listed in paragraphs below.

A second agent may be a B-cell-associated molecule. Other B-cell-associated molecules contemplated by the invention include binding molecules which bind to B-cell surface molecules that are not CD37 or CD20. B-cell-associated molecules, include but are not limited to, CD19 (B-lymphocyte antigen CD19, also referred to as B-lymphocyte surface antigen B4, or Leu-12), CD21, CD22 (B-cell receptor CD22, also referred to as Leu-14, B-lymphocyte cell adhesion molecule, or BL-CAM), CD23, CD40 (B-cell surface antigen CD40, also referred to as Tumor Necrosis Factor receptor superfamily member 5, CD40L receptor, or Bp50), CD80 (T lymphocyte activation antigen CD80, also referred to as Activation B7-1 antigen, B7, B7-1, or BB1), CD86 (T lymphocyte activation antigen CD86, also referred to as Activation B7-2 antigen, B70, FUN-1, or BU63), CD137 (also referred to as Tumor Necrosis Factor receptor superfamily member 9), CD152 (also referred to as cytotoxic T-lymphocyte protein 4 or CTLA-4), L6 (Tumor-associated antigen L6, also referred to as Transmembrane 4 superfamily member 1, Membrane component surface marker 1, or M3S1), CD30 (lymphocyte activation antigen CD30, also referred to as Tumor Necrosis Factor receptor superfamily member 8, CD30L receptor, or Ki-1), CD50 (also referred to as Intercellular adhesion molecule-3 (ICAM3), or ICAM-R), CD54 (also referred to as Intercellular adhesion molecule-1 (ICAM1), or Major group rhinovirus receptor), B7-H1 (ligand for an immunoinhibitory receptor expressed by activated T cells, B-cells, and myeloid cells, also referred to as PD-L1; see Dong, et al., “B7-H1, a third member of the B7 family, co-stimulates T-cell proliferation and interleukin-10 secretion,” Nat. Med., 5:1365-1369 (1999), CD134 (also referred to as Tumor Necrosis Factor receptor superfamily member 4, OX40, OX40L receptor, ACT35 antigen, or TAX-transcriptionally activated glycoprotein 1 receptor), 41 BB (4-1 BB ligand receptor, T-cell antigen 4-1 BB, or T-cell antigen ILA), CD153 (also referred to as Tumor Necrosis Factor ligand superfamily member 8, CD30 ligand, or CD30-L), CD154 (also referred to as Tumor Necrosis Factor ligand superfamily member 5, TNF-related activation protein, TRAP, or T cell antigen Gp39) and Toll receptors. The above list of construct targets and/or target antigens is exemplary only and is not exhaustive.

Cytokines and growth factors are second agents contemplated by the invention and include, without limitation, one or more of TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, G-CSF, Meg-CSF, GM-CSF, thrombopoietin, stem cell factor, and erythropoietin. Pharmaceutical compositions in accordance with the invention may also include other known angiopoietins, for example Ang-1, Ang-2, Ang-4, Ang-Y, and/or the human angiopoietin-like polypeptide, and/or vascular endothelial growth factor (VEGF). Growth factors for use in pharmaceutical compositions of the invention include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor α, cytokine-induced neutrophil chemotactic factor 1, cytokine-induced neutrophil chemotactic factor 2a, cytokine-induced neutrophil chemotactic factor 2β, β endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor α1, glial cell line-derived neutrophic factor receptor α2, growth related protein, growth related protein α, growth related protein β, growth related protein y, heparin binding epidermal growth factor, hepatocyte growth factor, hepatocyte growth factor receptor, insulin-like growth factor β, insulin-like growth factor receptor, insulin-like growth factor II, insulin-like growth factor binding protein, keratinocyte growth factor, leukemia inhibitory factor, leukemia inhibitory factor receptor α, nerve growth factor, nerve growth factor receptor, neurotrophin-3, neurotrophin-4, placenta growth factor, placenta growth factor 2, platelet derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor, stem cell factor receptor, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, vascular endothelial growth factor, and chimeric proteins and biologically or immunologically active fragments thereof.

Examples of chemotherapeutic agents contemplated as second agents include, but are not limited to, alkylating agents, such as nitrogen mustards (e.g., mechlorethamine, cyclophosphamide, ifosfamide, melphalan, and chlorambucil); nitrosoureas (e.g., carmustine (BCNU), lomustine (CCNU), and semustine (methyl-CCNU)); ethyleneimines and methyl-melamines (e.g., triethylenemelamine (TEM), triethylene thiophosphoramide (thiotepa), and hexamethylmelamine (HMM, altretamine)); alkyl sulfonates (e.g., buslfan); and triazines (e.g., dacabazine (DTIC)); antimetabolites, such as folic acid analogs (e.g., methotrexate, trimetrexate, and pemetrexed (multi-targeted antifolate)); pyrimidine analogs (such as 5-fluorouracil (5-FU), fluorodeoxyuridine, gemcitabine, cytosine arabinoside (AraC, cytarabine), 5-azacytidine, and 2,2′-difluorodeoxycytidine); and purine analogs (e.g, 6-mercaptopurine, 6-thioguanine, azathioprine, 2′-deoxycoformycin (pentostatin), erythrohydroxynonyladenine (EHNA), fludarabine phosphate, 2-chlorodeoxyadenosine (cladribine, 2-CdA)); Type I topoisomerase inhibitors such as camptothecin (CPT), topotecan, and irinotecan; natural products, such as epipodophylotoxins (e.g., etoposide and teniposide); and vinca alkaloids (e.g., vinblastine, vincristine, and vinorelbine); anti-tumor antibiotics such as actinomycin D, doxorubicin, and bleomycin; radiosensitizers such as 5-bromodeozyuridine, 5-iododeoxyuridine, and bromodeoxycytidine; platinum coordination complexes such as cisplatin, carboplatin, and oxaliplatin; substituted ureas, such as hydroxyurea; and methylhydrazine derivatives such as N-methylhydrazine (M1H) and procarbazine.

Non-limiting examples of chemotherapeutic agents, radiotherapeutic agents and other active and ancillary agents are also shown in Table 1.

TABLE 1

Alkylating agents
Nitrogen mustards
mechlorethamine
cyclophosphamide
ifosfamide
melphalan
chlorambucil
Nitrosoureas
carmustine (BCNU)
lomustine (CCNU)
semustine (methyl-CCNU)
Ethylenemine/Methyl-melamine
thriethylenemelamine (TEM)
triethylene thiophosphoramide
(thiotepa)
hexamethylmelamine
(HMM, altretamine)
Alkyl sulfonates
busulfan
Triazines
dacarbazine (DTIC)
Antimetabolites
Folic Acid analogs
methotrexate
Trimetrexate
Pemetrexed
(Multi-targeted antifolate)
Pyrimidine analogs
5-fluorouracil
fluorodeoxyuridine
gemcitabine
cytosine arabinoside
(AraC, cytarabine)
5-azacytidine
2,2′-difluorodeoxy-cytidine
Purine analogs
6-mercaptopurine
6-thioguanine
azathioprine
2′-deoxycoformycin
(pentostatin)
erythrohydroxynonyl-adenine (EHNA)
fludarabine phosphate
2-chlorodeoxyadenosine
(cladribine, 2-CdA)
Type I Topoisomerase Inhibitors
camptothecin
topotecan
irinotecan
Biological response modifiers
G-CSF
GM-CSF
Differentiation Agents
retinoic acid derivatives
Hormones and antagonists
Adrenocorticosteroids/antagonists
prednisone and equivalents
dexamethasone
ainoglutethimide
Progestins
hydroxyprogesterone caproate
medroxyprogesterone acetate
megestrol acetate
Estrogens
diethylstilbestrol
ethynyl estradiol/equivalents
Antiestrogen
tamoxifen
Androgens
testosterone propionate
fluoxymesterone/equivalents
Antiandrogens
flutamide
gonadotropin-releasing
hormone analogs
leuprolide
Nonsteroidal antiandrogens
flutamide
Natural products
Antimitotic drugs
Taxanes
paclitaxel
Vinca alkaloids
vinblastine (VLB)
vincristine
vinorelbine
Taxotere ® (docetaxel)
estramustine
estramustine phosphate
Epipodophylotoxins
etoposide
teniposide
Antibiotics
actimomycin D
daunomycin (rubido-mycin)
doxorubicin (adria-mycin)
mitoxantroneidarubicin
bleomycin
splicamycin (mithramycin)
mitomycinC
dactinomycin
aphidicolin
Enzymes
L-asparaginase
L-arginase
Radiosensitizers
metronidazole
misonidazole
desmethylmisonidazole
pimonidazole
etanidazole
nimorazole
RSU 1069
EO9
RB 6145
SR4233
nicotinamide
5-bromodeozyuridine
5-iododeoxyuridine
bromodeoxycytidine
Miscellaneous agents
Platinium coordination complexes
cisplatin
Carboplatin
oxaliplatin
Anthracenedione
mitoxantrone
Substituted urea
hydroxyurea
Methylhydrazine derivatives
N-methylhydrazine (MIH)
procarbazine
Adrenocortical suppressant
mitotane (o,p′-DDD)
ainoglutethimide
Cytokines
interferon (α, β, γ)
interleukin-2
Photosensitizers
hematoporphyrin derivatives
Photofrin ®
benzoporphyrin derivatives
Npe6
tin etioporphyrin (SnET2)
pheoboride-a
bacteriochlorophyll-a
naphthalocyanines
phthalocyanines
zinc phthalocyanines
Radiation
X-ray
ultraviolet light
gamma radiation
visible light
infrared radiation
microwave radiation

Second agents contemplated by the invention for treatment of autoimmune diseases are referred to as immunosuppressive agents, which act to suppress or mask the immune system of the individual being treated. Immunosuppressive agents include, for example, non-steroidal anti-inflammatory drugs (NSAIDs), analgesics, glucocorticoids, disease-modifying antirheumatic drugs (DMARDs) for the treatment of arthritis, or biologic response modifiers. Compositions in the DMARD description are also useful in the treatment of many other autoimmune diseases aside from RA.

Exemplary NSAIDs are chosen from the group consisting of ibuprofen, naproxen, naproxen sodium, Cox-2 inhibitors such as Vioxx and Celebrex, and sialylates. Exemplary analgesics are chosen from the group consisting of acetaminophen, oxycodone, tramadol of proporxyphene hydrochloride. Exemplary glucocorticoids are chosen from the group consisting of cortisone, dexamethasone, hydrocortisone, methylprednisolone, prednisolone, or prednisone. Exemplary biological response modifiers include, but are not limited to, molecules directed against cell surface markers (e.g., CD4, CD5, etc.), cytokine inhibitors, such as the TNF antagonists (e.g. etanercept (Enbrel), adalimumab (Humira) and infliximab (Remicade)), chemokine inhibitors and adhesion molecule inhibitors. The biological response modifiers include monoclonal antibodies as well as recombinant forms of molecules. Exemplary DMARDs include, but are not limited to, azathioprine, cyclophosphamide, cyclosporine, methotrexate, penicillamine, leflunomide, sulfasalazine, hydroxychloroquine, Gold [oral (auranofin) and intramuscular] and minocycline.

It is contemplated the binding molecule composition and the second agent may be given simultaneously in the same formulation. Alternatively, the agents are administered in a separate formulation but concurrently, with concurrently referring to agents given within 30 minutes of each other.

In another aspect, the second agent is administered prior to administration of the binding molecule composition. Prior administration refers to administration of the second agent within the range of one week prior to treatment with the antibody, up to 30 minutes before administration of the antibody. It is further contemplated that the second agent is administered subsequent to administration of the binding molecule composition. Subsequent administration is meant to describe administration from 30 minutes after antibody treatment up to one week after antibody administration.

It is further contemplated that when the binding molecule is administered in combination with a second agent, wherein the second agent is a cytokine or growth factor, or a chemotherapeutic agent, the administration may also include use of a radiotherapeutic agent or radiation therapy. The radiation therapy administered in combination with an antibody composition is administered as determined by the treating physician, and at doses typically given to patients being treated for cancer.

The amounts of binding molecule in a given dose will vary according to the size of the individual to whom the therapy is being administered as well as the characteristics of the disorder being treated. In exemplary treatments, it may be necessary to administer about 1 mg/day, about 5 mg/day, about 10 mg/day, about 20 mg/day, about 50 mg/day, about 75 mg/day, about 100 mg/day, about 150 mg/day, about 200 mg/day, about 250 mg/day, about 500 mg/day or about 1000 mg/day. The doses may also be administered based on weight of the patient, at a dose of about 0.01 to about 50 mg/kg. In a related embodiment, the binding molecule may be administered in a dose range of about 0.015 to about 30 mg/kg. In an additional embodiment, the binding molecule is administered in a dose of about 0.015, about 0.05, about 0.15, about 0.5, about 1.5, about 5, about 15 or about 30 mg/kg.

These compositions may be administered in a single dose or in multiple doses. Standard dose-response studies, first in animal models and then in clinical testing, reveal optimal dosages for particular disease states and patient populations.

The administration of the binding molecule composition decreases the B-cell population by at least 20% after a single dose of treatment. In one embodiment, the B-cell population is decreased by at least about 20, about 30, about 40, about 50, about 60, about 70, about 80, about 90 or about 100%. B-cell reduction is defined as a decrease in absolute B-cell count below the lower limit of the normal range. B-cell recovery is defined as a return of absolute B-cell count to either of the following: 70% of subject's baseline value or normal range.

The administration of CD20-specific binding molecules also results in enhanced apoptosis in particular B-cell subsets. Apoptosis refers to the induction of programmed cell death of a cell, manifested and assessed by DNA fragmentation, cell shrinkage, cell fragmentation, formation of membrane vesicles, or alteration of membrane lipid composition as assessed by annexin V staining.

Further, the administration of binding molecule compositions of the invention results in desired clinical effects in the disease or disorder being treated. For example, in patients affected by rheumatoid arthritis, in one aspect the administration improves the patient's condition by a clinically significant amount [e.g., achieves the American College of Rheumatology Preliminary Detection of Improvement (ACR20)], and/or an improvement of 20% in tender and swollen joint and 20% improvement in ⅗ remaining ACR measures (Felson et al., Arthritis Rheum. 1995, 38:727-35). Biological measures for improvement in an RA patient after administration of CD37-specific and CD20-specific binding molecules include measurement of changes in cytokine levels, measured via protein or RNA levels. Cytokines of interest include, but are not limited to, TNF-α, IL-1, interferons, Blys, and APRIL. Cytokine changes may be due to reduced B cell numbers or decreased activated T cells. In RA patients, markers relevant to bone turnover (bone resorption or erosion) are measured before and after administration of CD20-specific binding molecules. Relevant markers include, but are not limited to, alkaline phosphatase, osteocalcin, collagen breakdown fragments, hydroxyproline, tartrate-resistant acid phosphatase, and RANK ligand (RANKL). Other readouts relevant to the improvement of RA include measurement of C reactive protein (CRP) levels, erythrocyte sedimentation rate (ESR), rheumatoid factor, CCP (cyclic citrullinated peptide) antibodies and assessment of systemic B cell levels and lymphocyte count via flow cytometry. Specific factors can also be measured from the synovium of RA patients, including assessment of B cell levels in synovium from synovium biopsy, levels of RANKL and other bone factors and cytokines set out above.

In a related aspect, the effects of combination administration on other diseases is measured according to standards known in the art. For example, it is contemplated that Crohn's disease patients treated according to the invention achieve an improvement in Crohn's Disease Activity Index (CDAI) in the range of about 50 to about 70 units, wherein remission is at 150 units (Simonis et al, Scand. J. Gastroent. 1998, 33:283-8). A score of 150 or 200 is considered normal, while a score of 450 is considered a severe disease score. It is further desired that administration of the CD37-specific and CD20-specific binding molecules results in a reduction in perinuclear anti-neutrophil antibody (pANCA) and anti-Saccharomyces cervisiae antibody (ASCA) in individuals affected by inflammatory bowel disease.

It is further contemplated that adult and juvenile myositis patients treated according to the invention achieve an improvement in core set of evaluations, such as 3 out of 6 of the core set measured improved by approximately 20%, with not more than 2 of the core measurements worse by approximately 25% (see Rider et al., Arthritis Rheum. 2004, 50:2281-90).

It is further contemplated that SLE patients treated according to the invention achieve an improvement in Systemic Lupus Activity Measure (SLAM) or SLE Disease Activity Index (SLEDAI) score of at least 1 point (Gladman et al, J Rheumatol 1994, 21:1468-71) (Tan et al., Arthritis Rheum. 1982, 25:1271-7). A SLAM score of >5, or SLEDAI score>2, is considered clinically active disease. A response to treatment may be defined as improvement or stabilization over the in 2 disease activity measures (the SLE Disease Activity Index [SLEDAI] and the Systemic Lupus Activity Measure) and 2 quality of life measures (patient's global assessment and the Krupp Fatigue Severity Scale) (Petri et al., Arthritis Rheum. 2004, 50:2858-68.) It is further contemplated that administration of the binding molecule to SLE patients results in a reduction in anti-double-stranded DNA antibodies. Alternatively, improvement may be gauged using the British Isles Lupus Assessment Group Criteria (BILAG).

It is further contemplated that multiple sclerosis patients treated according to the invention achieve an improvement in clinical score on the Kurtzke Expanded Disability status scale (EDSS) (Kurtzke, F., Neurology 1983, 33:1444-52) of at least 0.5, or a delay in worsening of clinical disease of at least 1.0 on the Kurtzke scale (Rudick et al., Neurology 1997, 49:358-63).

It is further contemplated that patients suffering from IIM receiving CD37-specific and CD20-specific binding molecules achieve a reduction in at least one of five criteria set out in the Idiopathic Inflammatory Myopathy Criteria (IIMC) assessment (Miller, F., supra). It is further contemplated that administration to IIM patients results in a reduction in IIM associated factors selected from the group consisting of creatine kinase (CK), lactate dehydrogenase, aldolase, C-reactive protein, aspartate aminotransferase (AST), alanine aminotransferase (ALT), and antinuclear autoantibody (ANA), myositis-specific antibodies (MSA), and antibody to extractable nuclear antigens. Alternatively, patients meet 3 out of 6 of the criteria set out in Rider et al., Arthritis Rheum., 50(7):2281-2290 (2004), with worsening in no more than 2 criteria.

In some embodiments, patients suffering from a B cell cancer receive treatment according to the invention and demonstrate an overall beneficial response to the treatment, based on clinical criteria well-known and commonly used in the art, and as described below, such as a decrease in tumor size, decrease in tumor number and/or an improvement in disease symptoms.

Exemplary clinical criteria are provided by the U.S. National Cancer Institute (NCI), which has divided some of the classes of cancers into the clinical categories of “indolent” and “aggressive” lymphomas. Indolent lymphomas include follicular cell lymphomas, separated into cytology “grades,” diffuse small lymphocytic lymphoma/chronic lymphocytic leukemia (CLL), lymphoplasmacytoid/Waldenstrom's Macroglobulinemia, Marginal zone lymphoma and Hairy cell leukemia. Aggressive lymphomas include diffuse mixed and large cell lymphoma, Burkitt's lymphoma/diffuse small non-cleaved cell lymphoma, Lymphoblastic lymphoma, Mantle cell lymphoma and AIDS-related lymphoma. In some cases, the International Prognostic Index (IPI) is used in cases of aggressive and follicular lymphoma. Factors to consider in the IPI include age (<60 years of age versus>60 years of age), serum lactate dehydrogenase (levels normal versus elevated), performance status (0 or 1 versus 2-4) (see definition below), disease stage (I or II versus III or IV), and extranodal site involvement (0 or 1 versus 2-4). Patients with 2 or more risk factors have less than a 50% chance of relapse-free and overall survival at 5 years.

Performance status in the aggressive IPI is defined as follows: Grade Description: 0 Fully active, able to carry on all pre-disease performance without restriction; 1 Restricted in physically strenuous activity but ambulatory and able to carry out work of a light or sedentary nature, e.g., light house work, office work; 2 Ambulatory and capable of all selfcare but unable to carry out any work activities, up to and about more than 50% of waking hours; 3 Capable of only limited selfcare, confined to bed or chair more than 50% of waking hours; 4 Completely disabled, unable to carry on any selfcare, totally confined to bed or chair; and, 5 Dead. (See., The International Non-Hodgkin's Lymphoma Prognostic Factors Project. A predictive model for aggressive non-Hodgkin's lymphoma. N. Engl. J. Med. 329:987-94, 1993.)

Typically, the grade of lymphoma is clinically assessed using the criterion that low-grade lymphoma usually presents as a nodal disease and is often indolent or slow-growing. Intermediate- and high-grade disease usually presents as a much more aggressive disease with large extranodal bulky tumors.

The Ann Arbor classification system is also used to measure progression of tumors, especially non-Hodgkins lymphomas. In this system, stages I, II, III, and IV of adult NHL can be classified into A and B categories depending on whether the patient has well-defined generalized symptoms (B) or not (A). The B designation is given to patients with the following symptoms: unexplained loss of more than 10% body weight in the 6 months prior to diagnosis, unexplained fever with temperatures above 38° C. and drenching night sweats. Definitions of the stages are as follows: Stage I-involvement of a single lymph node region or localized involvement of a single extralymphatic organ or site. Stage II-involvement of two or more lymph node regions on the same side of the diaphragm or localized involvement of a single associated extralymphatic organ or site and its regional lymph nodes with or without other lymph node regions on the same side of the diaphragm. Stage III-involvement of lymph node regions on both sides of the diaphragm, possibly accompanying localized involvement of an extralymphatic organ or site, involvement of the spleen, or both. Stage 1V-disseminated (multifocal) involvement of one or more extralymphatic sites with or without associated lymph node involvement or isolated extralymphatic organ involvement with distant (non-regional) nodal involvement. For further details, see The International Non-Hodgkin's Lymphoma Prognostic Factors Project: A predictive model for aggressive non-Hodgkin's lymphoma, New England J. Med. (1993) 329:987-994.

In one aspect, a therapeutic effect of the methods according to the invention is determined by the level of response, for example a partial response is defined as tumor reduction to less than one-half of its original size. A complete response is defined as total elimination of disease confirmed by clinical or radiological evaluation. In one embodiment, the individual receiving treatment according to the invention demonstrates at least a partial response to treatment.

According to the Cheson criteria for assessing NHL developed in collaboration with the National Cancer Institute (Cheson et al., J Clin Oncol. 1999, 17:1244; Grillo-Lopez et al., Ann Oncol. 2000, 11:399-408), a complete response is obtained when there is a complete disappearance of all detectable clinical and radiographic evidence of disease and disease-related symptoms, all lymph nodes have returned to normal size, the spleen has regressed in size, and the bone marrow is cleared of lymphoma.

An unconfirmed complete response is obtained when a patient shows complete disappearance of the disease and the spleen regresses in size, but lymph nodes have regressed by more than 75% and the bone marrow is indeterminate. An unconfirmed complete response meets and exceeds the criteria for partial response. An overall response is defined as a reduction of at least 50 percent in overall tumor burden.

Similar criteria have been developed for various other forms of cancers or hyperproliferative diseases and are readily available to a person of skill in the art. See, e.g., Cheson et al., Clin Adv Hematol Oncol. 2006, 4:4-5, which describes criteria for assessing CLL; Cheson et al., J Clin Oncol. 2003, 21:4642-9, which describes criteria for AML; Cheson et al., Blood 2000, 96:3671-4, which describes criteria for myelodysplastic syndromes.

In another aspect, a therapeutic response in patients having a B cell cancer is manifest as a slowing of disease progression compared to patients not receiving therapy. Measurement of slowed disease progression or any of the above factors may be carried out using techniques well-known in the art, including bone scan, CT scan, gallium scan, lymphangiogram, MRI, PET scans, ultrasound, and the like.

It will also be apparent that dosing may be modified if traditional therapeutics are administered in combination with therapeutics of the invention.

As an additional aspect, the invention includes kits which comprise one or more compounds or compositions useful in the methods of the invention packaged in a manner which facilitates their use to practice methods of the invention. In a simplest embodiment, such a kit includes a compound or composition described herein as useful for practice of a method of the invention packaged in a container such as a sealed bottle or vessel, with a label affixed to the container or included in the package that describes use of the compound or composition to practice the method of the invention. Preferably, the compound or composition is packaged in a unit dosage form. The kit may further include a device suitable for administering the composition according to a preferred route of administration or for practicing a screening assay. The kit may include a label that describes use of the binding molecule composition(s) in a method of the invention.

The present invention also comprises articles of manufacture. Such articles comprise CD37-specific binding molecules or CD37-specific and CD20-specific binding molecules, optionally together with a pharmaceutical carrier or diluent, and at least one label describing a method of use of the binding molecules according to the invention. Such articles of manufacture may also optionally comprise at least one second agent for administration in connection with the binding molecules.

The present invention also calls for use of a composition comprising a CD37-specific binding molecule or CD37-specific and CD20-specific binding molecules in the manufacture of a medicament for the treatment or prophylaxis of a disease involving aberrant B-cell activity.

The invention includes the identification of the pharmacological properties of TRU-016, which were determined from studies performed with TRU-016 or its chimeric (mouse/human) progenitor, the SMIP-016 protein, as discussed previously herein. Comparative studies demonstrated that the TRU-016 was indistinguishable from SMIP-016 protein in terms of its strong binding specificity for B cells, its ability to kill B cell targets (without impacting T cells, NK cells, or monocytes) via apoptosis or Fc-dependent cell mediated cytotoxicity (FcDCC), and its efficacy in vivo in xenograft immunodeficient mouse models of Burkitt's lymphoma (Raji, Ramos, or Daudi cells lines) and follicular lymphoma (DOHH2).

Dose-response experiments identified effective doses of TRU-016 as well as doses that had minimal to no activity in vivo. Depending on the model, effective doses ranged from about 5.6 to about 9 mg/kg per injection, whereas doses of about 0.2 mg/kg to about 1.3 mg/kg per injection showed little to no significant anti-tumor activity. However, these doses should not be construed in any way to be limiting for the purposes of the invention. One of ordinary skill in the art will be able to determine a range of effective doses during the course of treatment depending on the tumor type, the age and weight of the patient, the stage of disease and other variables.

TRU-016 has several properties that are clinically beneficial when used to treat malignant human B cell tumors. First, because TRU-016 delivers its signal via interaction with CD37 rather than CD20, TRU-016 offers the possibility for therapeutic benefit even when CD20 is lost or removed from the surface of the targeted B cells (as has been reported for CLL) (Kennedy et al., Blood 101: 1071-1079, 2003; Jilani et al., Blood 102: 35514-3520, 2003). Second, TRU-016 has led to increased anti-tumor activity when combined with other therapeutic drugs used for B cell tumors. TRU-016 treatment of human B cell tumor lines increased apoptosis additively or synergistically in combination with chemotherapeutic drugs and when combined with rituximab, anti-CD37 SMIP treatment in vivo led to an increased anti-tumor activity in a xenograft model. In CLL, TRU-016 may work well in combination by mediating an apoptotic signal induced through a caspase-independent mechanism that is distinct from other therapeutic agents. Thus, TRU-016 as a single agent may provide clinical benefit when anti-CD20 or chemotherapy fail due to inactivation of the apoptotic pathway induced by these drugs.

TRU-016 supports strong FcDCC, but lacks the ability to induce CDC, against B cell targets. As it has been proposed that CDC is a major mechanism that contributes to “tumor lysis syndrome” associated with anti-CD20 (rituximab) treatment in CLL and diffuse large B-cell lymphomas (DLBCL) (van der Kolk et al., Br. J. Haematol. 115: 807-811, 2001; Gutierrez et al., Leuk. & Lymph. 47: 111-115, 2006) it is possible that patients receiving TRU-016 will experience less of this toxicity.

Although CD37 has been reported to be expressed at very low levels on T cells and monocytes, exposure of these cells to even relatively high concentrations of TRU-016 did not lead to their depletion from cultures of unfractionated peripheral blood mononuclear cells, nor did it induce or inhibit proliferation of T cells.

Accordingly, TRU-016 binds specifically to CD37, a B cell antigen found at high levels on normal and malignant human B cells. TRU-016 induces death of primary malignant CLL cells in vitro and of human B cell tumor xenografts in vivo, including in a model in which CD20-directed treatment failed over time. Therefore, TRU-016 is a promising therapeutic agent for treating CD37+ B cell malignancies and administration of TRU-016 in a human clinical setting for the treatment of patients with B cell lymphoma and leukemia who fail or relapse with the current standard of care is indicated.

The invention also includes methods of treating an individual having or suspected of having a relapse of a disease associated with aberrant B-cell activity, comprising administering to the individual one or more CD37-specific binding molecules described herein. Likewise, the invention includes methods of treating an individual who becomes resistant to CD20-directed therapies or who develops rituximab-refractory disease, comprising administering to the individual one or more CD37-specific binding molecules described herein.

In an exemplary aspect, the invention includes methods of treating a non-Burkitt's B cell malignancy, comprising administering to an individual in need thereof one or more CD37-specific binding molecules. Such CD37-specific binding molecules include all of the CD37-specific binding molecules described herein. The non-Burkitt's B cell malignancies include, but are not limited to, B-cell chronic lymphocytic leukemia (CLL)/small lymphocytic lymphoma, B-cell prolymphocytic leukemia, an acute lymphoblastic leukemia (ALL), lymphoplasmacytic lymphoma (including, but not limited to, Waldenstrom's macroglobulinemia), marginal zone lymphomas (including, but not limited to, splenic marginal zone B-cell lymphoma, nodal marginal zone lymphoma, and extranodal marginal zone B-cell lymphoma of mucosa-associated lymphoid tissue (MALT) type), hairy cell leukemia, plasma cell myeloma/plasmacytoma, follicular lymphoma, mantle cell lymphoma (MCL), diffuse large cell B-cell lymphoma, transforming large B cell lymphoma, mediastinal large B-cell lymphoma, intravascular large B-cell lymphoma, primary effusion lymphoma, and non-Burkitt's NHL.

### BRIEF DESCRIPTION OF THE DRAWING

FIG. 1A diagrams the structure of the TRU-016 molecule; FIG. 1B shows the results of SDS-PAGE analysis, demonstrating that the expressed protein migrates at a Mr of approximately 110 kDa under nonreducing conditions, and approximately 52 kDa when subjected to reducing conditions; and FIG. 1C shows that the TRU-016 molecule demonstrates high level, specific binding to human peripheral blood B lymphocytes, and a much lower level of binding to other subpopulations of cells in the non-B cell lymphocyte gate (CD1 g negative population) when analyzed by flow cytometry.

FIG. 2A-E shows binding inhibition by different CD37 targeted reagents.

FIG. 3A demonstrates FITC C1q binding to TRU-016 molecular forms incubated with Ramos B Cells in normal human serum with and without cobra venom factor (CVF); FIG. 3B shows CDC activity of TRU-016 molecular forms incubated with Ramos B Cells in normal human serum with and without CVF; and FIG. 3C shows CDC activity of TRU-016 molecular forms incubated with Ramos B cells and human or rabbit complement.

FIG. 4 shows the HPLC size exclusion chromatography (SEC) traces obtained from GPC purification of the TRU-016, plotting absorbance versus retention time for the different fractions collected.

FIG. 5A shows the binding properties of SEC fractions; FIG. 5B shows the CDC activity of SEC fractions; and FIG. 5C shows the ADCC activity of SEC fractions.

FIG. 6 shows the CDC activity of TRU-015, rituxan, TRU-016, or a combination thereof on Ramos B cells.

FIG. 7 shows that the effect of TRU-016 on CDC activity of TRU-015 on DHL-4 B cells.

FIG. 8 shows the effect of TRU-016 on the CDC activity of TRU-015 and rituxan.

FIG. 9 shows the effect of TRU-016 on TRU-015 in a CDC assay.

FIG. 10 shows the effect of TRU-016 on rituxan in a CDC assay.

FIG. 11 shows the interaction of TRU-015 and TRU-016 in an ADCC assay using BJAB cells.

FIG. 12 shows the interaction of TRU-015 and TRU-016 in an ADCC assay using Daudi cells.

FIG. 13 shows the interaction of TRU-015 and TRU-016 in an ADCC assay using Ramos cells.

FIG. 14 shows the effect of rituxan, TRU-016, and a combination thereof on the specific killing of BJAB cells.

FIG. 15 shows the effect of rituxan, TRU-016, and a combination thereof on the specific killing of BJAB cells.

FIG. 16 shows the effect of TRU-015, TRU-016, and a combination thereof on the specific killing of BJAB cells.

FIG. 17 shows the effect of TRU-015, TRU-016, and a combination thereof on the specific killing of BJAB cells.

FIG. 18A-D shows that TRU-016 dimer forms do not mediate CDC alone, but potentiate the CDC activity of Rituximab in vitro.

FIG. 19A-B demonstrates that protein A purified TRU-016 induces apoptosis of Ramos and Daudi cells, while dimer forms require crosslinking.

FIG. 20 shows that TRU-016 preferentially depletes normal B cells from PBMC cultures.

FIG. 21 demonstrates the efficacy of TRU-016 compared to huIgG, rituxan, and the combination treatment of TRU-016 and rituxan on tumor volume in animals.

FIGS. 22A and B shows that TRU-016 dimer forms exhibit significant anti-tumor activity, as measured by effect on tumor volume and percent survival in a mouse xenograft tumor model.

FIG. 23 demonstrates that TRU-016 dimers do not augment CDC activity resulting from treatment with MHCII, CD19, CD80/86, or CD45 specific reagents.

FIG. 24 shows the percent survival of mice with Ramos tumors (up to 90 days) after treatment with TRU-016, rituximab, or a combination thereof.

FIGS. 25 and 26 show the percent survival of mice with Daudi tumors (up to 90 days) after treatment with TRU-016 or rituximab.

FIG. 27 shows that TRU-016 effectively reduced relative cell viability in cells treated with fludarabine, thereby potentiating the cytotoxic effect of fludarabine alone.

FIG. 28 shows that TRU-016 induced greater cell toxicity than herceptin or rituximab in rituximab-resistant cell lines.

FIG. 29 shows that TRU-016 induced tyrosine phosphorylation in CD19+ primary CLL B cells.

FIG. 30A shows the consensus amino acid sequence of humanized TRU-016 construct no. 019001 (SEQ ID NO: 6) and TRU-016 (SEQ ID NO: 2) with Kabat numbering; FIG. 30B shows amino acid sequence alignments of three humanized TRU-16 constructs (019001, 019008, and 109009).

FIG. 31 shows the DNA and amino acid sequence alignments of three humanized constructs of TRU-016 (019001, 019041, and 019044).

FIG. 32 shows the FASTA formatted sequence alignments of the same three humanized constructs of TRU-016 (019001, 019041, and 019044).

FIG. 33 demonstrates that TRU-016 acts in synergy with standard chemotherapeutic agents to kill mantle cell lymphoma (MCL) cells, Rec-1 cells.

FIG. 34 shows that TRU-016 was statistically superior to Rituxan in the in vivo treatment of an animal model of follicular lymphoma as shown by survival rate (FIG. 34A) and tumor-free percentage (FIG. 34B).

### EXAMPLES

Additional aspects and details of the invention will be apparent from the following examples, which are intended to be illustrative rather than limiting. Example 1 describes the production of a CD37-specific binding molecule; Example 2 demonstrates that TRU-016 and various CD37-specific antibodies recognize the same or overlapping epitopes; Example 3 shows that TRU-016 is deficient in binding C1q and activating the classical complement activation pathway; Example 4 demonstrates activity and binding of TRU-016 multimers; Example 5 describes the production of a CD20-specific binding molecule; Example 6 shows that combinations of TRU-016 with TRU-015 or rituxan synergistically increase apoptosis in B cells; Example 7 shows that combinations of TRU-016 with CD20-specific antibodies or SMIPs synergistically increase CDC; Example 8 demonstrates that TRU-016 augments the ADCC and the CDC activity of CD20-specific antibodies and SMIPS; Example 9 demonstrates that TRU-016 induces apoptosis in B cells; Example 10 shows that combinations of a CD37-specific SMIP with a CD20-specific antibody synergistically reduce tumor volume in a murine tumor xenograft model; Example 11 shows that a CD37-specific SMIP alone also reduces tumor volume in a murine tumor xenograft model; Example 12 demonstrates that TRU-016 does not affect the CDC activity of other B cell surface receptors; Example 13 demonstrates that TRU-016 does not augment the CDC activity of various targeted receptors, including MHCII, CD19, CD80/86, and CD40; Example 14 provides additional data showing that TRU-016 increases survival in vivo in mice with tumors; Example 15 demonstrates that TRU-016 potentiates fludarabine-induced cell death in CLL cells in vitro; Example 16 shows that TRU-016 induces direct cytotoxicity in rituximab-resistant cells; Example 17 shows that TRU-016 induces tyrosine phosphorylation in CD19+ primary CLL B cells; Example 18 provides humanized TRU-016 molecules; Example 19 shows a dose response study of TRU-016 in an established subcutaneous human tumor (DOHH2) xenograft model in mice; Example 20 demonstrates the efficacy of TRU-016 and rituxan as single agents in the human tumor (DOHH2) xenograft model; Example 21 provides results from an in vitro evaluation of the effect of TRU-016 in combination with chemotherapeutic agents; Example 22 sets out a study of TRU-016 in the treatment of various refractory B cell diseases; and Example 23 describes the use of TRU-016 in the treatment of relapse or in rituximab-refractory disease.

### Example 1

### Production of a CD37-Specific Binding Molecule

CD37-specific SMIPs are described in co-owned U.S. application Ser. No. 10/627,556 and U.S. Patent Publication Nos. 2003/133939, 2003/0118592 and 2005/0136049. An exemplary SMIP, TRU-016, is produced as described below.

TRU-016 [G28-1 scFv VH11S (SSC-P)H WCH2 WCH3] is a recombinant single chain protein that binds to the CD37 antigen. The binding domain was based on the G28-1 antibody sequence previously disclosed in the patent publications listed in the preceding paragraph, which disclosure is incorporated herein by reference. The binding domain is connected to the effector domain, the CH2 and CH3 domains of human IgG1, through a modified hinge region. TRU-016 exists as a dimer in solution and the dimer has a theoretical molecular weight of approximately 106,000 daltons.

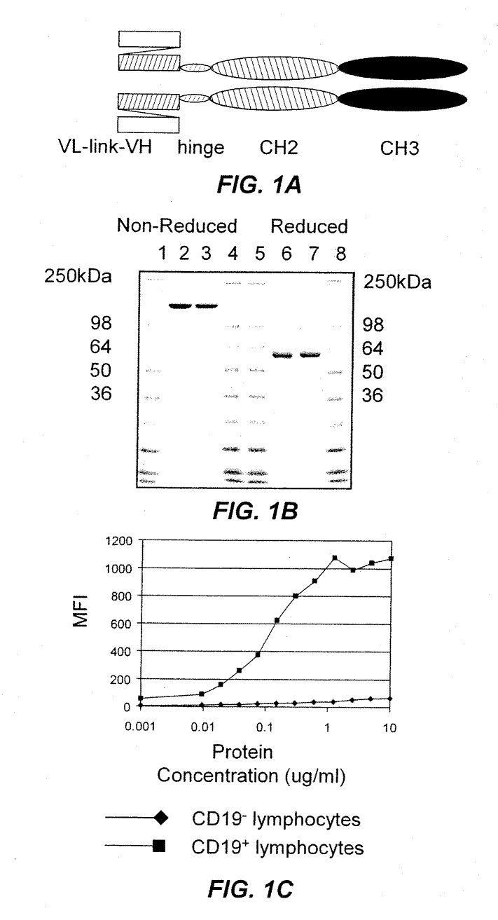

Total RNA from the G28-1 hybridoma was isolated using Trizol RNA (Gibco) reagent according to the manufacturer's instructions. cDNA was prepared using 5 μg RNA, random primers and Superscript II Reverse Transcriptase (GIBCO BRL). The variable domains were cloned using pools of degenerate primers for the different murine VK or VH gene families. The variable domains from the G28-1 hybridoma were cloned into PCR 2.1 TOPO cloning vectors (Invitrogen) and DNA from transformants with correct size inserts was sequenced. Heavy and light chain variable regions from correct clones were then used as templates for sewing PCR amplification of a G28-1 scFv joined together in the VL-VH orientation with a 15 aa (gly4ser)₃ linker. The anti-CD37 scFv was attached to a modified human IgG1 hinge, CH2, and CH3 domains (see FIG. 1A). In order to ensure adequate expression by mammalian cells, modifications of the variable regions were selected that allowed significant increases in expression by mammalian cells. Specifically, a leucine was changed to a serine at position 11 of the scFV. The predicted mature peptide is 473 amino acids long.

The polynucleotide sequence encoding TRU-016 and the amino acid sequence of TRU-016 are respectively set out in SEQ ID NOs: 1 and 2.

TRU-016 was produced by recombinant DNA technology in a Chinese hamster ovary (CHO) mammalian cell expression system. Transfected CHO cells that produce the SMIP were cultured in a bioreactor using proprietary media.

TRU-016 SMIPs were purified from CHO culture supernatants by Protein A affinity chromatography. Using dPBS, a 50 mL rProtein A FF sepharose column (GE Healthcare rProtein A Sepharose FF, Catalog #17-0974-04) was equilibrated at 5.0 m/s/min (150 cm/hr) for 1.5 column volumes (CV). The culture supernatant was loaded to the rProtein A Sepharose FF column at a flow rate of 1.7 mls/min using the AKTA Explorer 100 Air (GE healthcare AKTA Explorer 100 Air, Catalog #18-1403-00), capturing the recombinant TRU-016. The column was washed with dPBS for 5 Column Volumes (CV), then 1.0 M NaCl, 20 mM Sodium Phosphate, pH 6.0, and then with 25 mM NaCl, 25 mM NaOAc, pH 5.0. These washing steps removed nonspecifically bound CHO host cell proteins from the rProtein A column that contribute to product precipitation after elution.

The recombinant TRU-016 was eluted from the column with 100 mM Glycine, pH 3.5.10 mL fractions of the eluted product were recovered and the eluted product was then brought to pH 5.0 with 20% of the eluted volume of 0.5 M 2-(N-Morpholino)ethanesulfonic acid (MES) pH6.0. This eluted product was prepared for GPC purification by concentration of the sample to approximately 25 mg/mL TRU-016 and then filter sterilized in preparation for GPC purification.

Purified protein was then subjected to GPC size exclusion chromatography (SEC) to achieve further purification of the TRU-016 (dimer) molecule from higher molecular weight aggregates. Using dPBS, an XK 50/100 column (GE healthcare XK 50/100 empty chromatography column, Catalog #18-8753-01) containing 1 L of Superdex 200 FF sepharose was equilibrated at 12.6 mls/min (38 cm/hr) for 1.5 column volumes (CV). A maximum volume of 54 mls (3% CV) of sample was applied to the column. The column continued to run at 12.6 ml/min and the eluted protein was fractionated in 40 mL fractions. Each fraction was analyzed for product quality using an analytic HPLC, and the eluted fractions were pooled for >95% POI (non-aggregated) TRU-016. This resultant pool was filter sterilized at 0.22 μm. The material was then concentrated and formulated with 20 mM sodium phosphate and 240 mM sucrose, with a resulting pH of 6.0. The composition is filtered before filling into glass vials at a concentration of 10 mg/mL. Each glass vial contains 5 mL of TRU-016 (50 mg/vial).

TRU-016 protein was also subject to SDS-PAGE analysis on 4-20% Novex Tris-glycine gels (Invitrogen, San Diego, Calif.). Samples were loaded using Novex Tris-glycine SDS sample buffer (2×) under reducing (addition of 1/10 volume NuPAGE sample reducing agent) or non-reducing conditions after heating at 95° C. for 3 minutes, followed by electrophoresis at 150V for 90 minutes. Electrophoresis was performed using 1× Novex Tris-Glycine SDS Running Buffer (Invitrogen). Gels were stained after electrophoresis in Coomassie SDS PAGE R-250 stain for 30 minutes with agitation, and destained for at least one hour. The predicted molecular weight of the mature peptide is 51.5 kDa. Under reducing conditions, fusion protein migrates at the expected molecular weight. Under non-reducing conditions, the molecule migrates at approximately 150 kDa (FIG. 1B).

Experiments were also performed to determine that the binding specificity of the parent antibody to the CD37 cell surface receptor is preserved in TRU-016. Human PBMCs were isolated over LSM density gradients and incubated with unconjugated TRU-016 and PE-conjugated anti-human CD19. Cells were washed and incubated with 1:100 FITC GAH IgG (Fc specific) for 45 minutes on ice. Cells were washed and analyzed by two-color flow cytometry on a FACsCalibur instrument using Cell Quest software. Cells were gated for B lymphocytes or non-B lymphocytes by CD19 staining.

With increasing concentrations of TRU-016, the FITC signal on the B lymphocyte (CD1 g positive gate) increased rapidly from 0.01-1.0 μg/ml, until it reached saturation at approximately 1 μg/mL or a mean fluorescence intensity (MFI) of 1000. In contrast, the staining of the non-B lymphocyte population is detectable, but very low, and increases slowly with increasing concentration of scFvIg. Thus, the staining pattern of the G28-1 murine monoclonal antibody is preserved with TRU-016 (FIG. 1C).

The CD37-binding molecules according to the invention describe structures (binding domains derived from antibodies, hinge variants, CH2CH3 regions being the same or different, and various isotypes).

### Example 2

### TRU-016 and Various CD37-Specific Antibodies Bind the Same or Overlapping Epitopes on CD37

Experiments were performed to identify the CD37 epitope bound by TRU-016 and other previously described CD37-specific antibodies.

Unconjugated MB371 (#555457) and FITC-conjugated MB371 (#555456) were obtained from BD Pharmingen (San Jose, Calif.), FITC-conjugated BL14 (#0457) from Immunotech/Beckman Coulter (Fullerton, Calif.), FITC-conjugated NMN46 (#RDI-CBL 136FT) and unconjugated NMN46 (#RDI-CBL 136) from RDI (Flanders, N.J.), FITC-conjugated IPO24 (#186-040) and unconjugated IPO-24 (#186-020) from Ancell Corporation (Bayport, Minn.), FITC-conjugated HHI (#3081) and unconjugated HH1 (#3080) from DiaTec.Com (Oslo, Norway) and FITC-conjugated WR17 (YSRTMCA483F) and unconjugated WR17 (YSRTMCA483S) from Accurate Chemical & Scientific (Westbury, N.Y.). TRU-016 protein was produced as described in Example 1.

TRU-016 was conjugated to FITC at Trubion using a Molecular Probes Fluororeporter FITC Labeling Kit (F6434) according to manufacturer's instructions as follows: TRU-016 protein peak of interest (POI) at 13.5 mg/mL was adjusted to 5 mg/mL with PBS. 1 mg (200 ul) was added to kit tubes with a stirbar, and 1 M NaHCO3 (adjusted to pH 8.5 with 6N NaOH), was added to a final concentration of 0.1 M. 50 ul DMSO was added to 370 ug of FITC and was added to the tubes at molar ratios of 15, 20, 30 and 40 FITC:protein using the following formula to determine the ul of FITC to add: [ul of FITC solution to add=5 mg/mL protein×0.2 mL×389×100× desired molar ratio/Molecular weight of TRU-016 (110,000)].

Reactions were shielded from light and stirred continuously for 75 minutes at room temperature. Reactions were added to spin columns prepared as described in the kit and spun at 1100 g for 5 minutes to buffer exchange into PBS with azide and remove unconjugated FITC. The OD at 280 nM and 494 nM was determined with 2 ul drops on the Nanodrop; the extinction coefficient for TRU-016 was experimentally determined for this instrument by reading dilutions of the starting unconjugated SMIP, the concentration of each of the conjugates was 4.25 mg/ml and the following FITC:protein rations were determined: 2.7 FITC/TRU-016 at a ratio of 15; 3.7 FITC/TRU-016 at a ratio of 20; 4.4 FITC/TRU-016 at a ratio of 30; and 5.1 FITC/TRU-016 at a ratio of 40.

BSA was added to 3 mg/mL to help stabilize the protein. Binding of each fraction was assessed at dilutions ranging from 100-24,300× on Ramos and 3200-25,600 on human PBMC. All bound, but the MR30 ratio was chosen for further use since it gave a high MFI that was well maintained over the titration range used, indicating that binding avidity was least affected in this reaction.

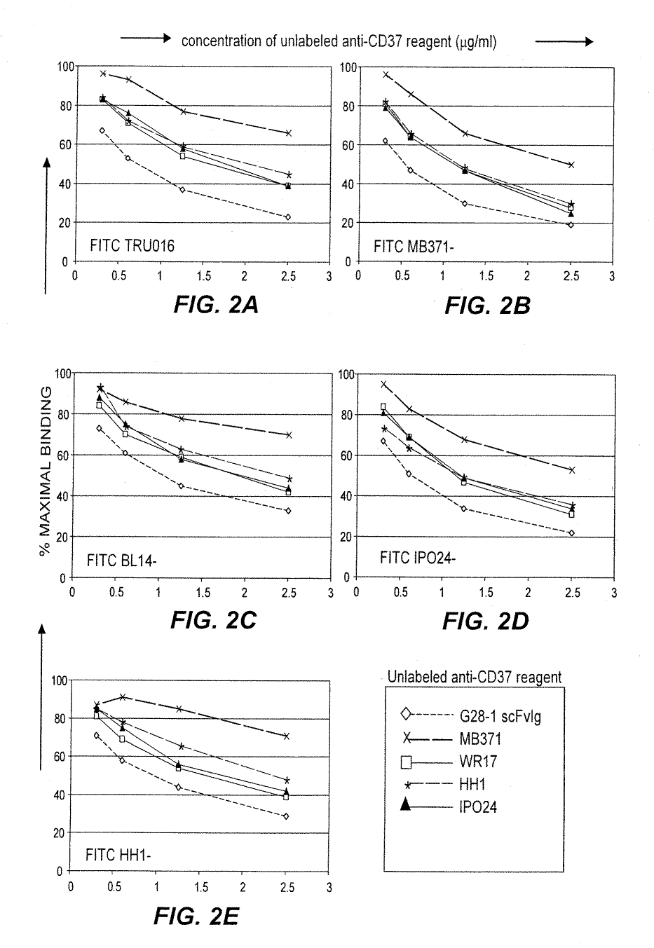

FITC labeled antibody conjugates were titrated from 10 ng/mL to 10 μg/mL in an initial binding study to determine the optimal amounts to use in the blocking studies. The level chosen was just below saturating amounts, and was kept constant in the subsequent assays, while levels of blocking antibody were increased over a 10-fold range. Data were plotted as percent of maximal binding versus concentration of blocking antibody, so that higher levels indicate less efficient blocking, while lower levels indicate more efficient blocking activity. All of the antibodies tested showed blocking activity of the maximal binding observed without unlabeled reagents (FIG. 2).

BJAB-cells, a B lymphoblastoid B-cell line, (courtesy of Ed Clark, University of Washington) were then stained with a panel of various clones of anti-CD37 MAbs, including MB371, BL14, NMN46, IPO24, HH1, WR17, and the TRU-016 SMIP.

For competitive binding assays, 2.5×10⁵ BJAB cells were incubated in 96-well V-bottom plates in staining media (PBS with 2% mouse sera) with the FITC-conjugated anti-CD37 MAbs at 1.25 μg/mL in the presence of unconjugated anti-CD37 MAb at the indicated concentrations (2.5, 1.25, 0.6, or 0.3 μg/ml) or staining media for 45 minutes on ice in the dark. Blocking antibodies and FITC labeled antibody conjugates were added to reactions prior to addition of cells. The cells were then washed 2½ times with PBS and fixed with 1% paraformaldehyde (# 19943, USB, Cleveland, Ohio). The cells were analyzed by flow cytometry using a FACsCalibur instrument and CellQuest software (BD Biosciences, San Jose, Calif.).

For FACs cross blocking assays, 2.5×10⁵ BJAB cells were incubated in 96-well V-bottom plates in staining media (PBS with 2% mouse sera) in the presence of unconjugated anti-CD37 MAb at 5 μg/mL staining media for 45 minutes at room temperature in the dark. FITC-conjugated anti-CD37 MAbs were then added to a final concentration of 2 ug/ml, resulting in a dilution of the unlabelled reagents to 3.3 μg/ml. The reactions were then further incubated for 45 minutes at room temperature in the dark. Reactions were washed 2.5 times with PBS and fixed in 1% paraformaldehyde in PBS (#19943, USB, Cleveland, Ohio). Cells were analyzed by flow cytometry on a FACsCalibur instrument using Cell Quest software (BD Biosciences, San Jose, Calif.).

For cell binding assays, cells were suspended in PBS (#14040-133, Gibco/Invitrogen, Grand Island N.Y.) containing 2% FBS (#16140-071, Gibco/Invitrogen, Grand Island, N.Y.), (staining media) at a concentration of approximately 4×10⁶ cells/mL. Cells were then plated and test samples, diluted in staining media, were then added 1:1 to the final designated concentrations. Reactions were incubated for 45 minutes on ice. Samples were centrifuged and washed 2 times with PBS. FITC goat anti-human IgG (#H10501, CalTag, Burlingame Calif.) was added at a final dilution of 1:50, and incubated 45 minutes on ice. Samples were centrifuged, washed in PBS, then fixed in 200 μl 1% paraformaldehyde in PBS (#19943, USB, Cleveland, Ohio). Cells were analyzed by flow cytometry on a FACs Calibur instrument using Cell Quest software (BD Biosciences, San Jose, Calif.).

Each antibody showed dose dependent inhibition of binding, indicating that all the molecules tested bind to an identical or closely related epitope. A different potency for inhibition of binding was observed for each antibody. TRU-016 SMIP had the highest level of blocking activity of all molecules tested, while HH1 gave an intermediate level of blocking activity, and WR17, IPO24 blocked better than MB371, but showed less effective blocking than the other two unlabeled molecules (FIG. 2).

In addition to analysis of blocking activity, a similar series of experiments was performed in which various CD37 targeted antibodies were tested for their ability to compete with one another for binding to the CD37 receptor. The results from these experiments, like results obtained in the blocking studies for all the molecules tested, indicated that the various CD37 targeted antibodies and TRU-016 have the same or closely overlapping epitopes.

### Example 3

### TRU-016 is Deficient in Binding C1q and Activating the Classical Complement Activation Pathway

Experiments were performed to explore why the TRU-016 dimer peak fails to mediate significant levels of complement dependent killing of B cell targets. One possibility was that TRU-016 dimer shows reduced binding to components of the complement cascade relative to normal human IgG1 antibody. Thus, experiments were performed to determine if TRU-016 activates the classical complement activation pathway by looking for TRU-016 binding to C1q. C1q, is a subunit of the C1 enzyme complex that activates the serum complement system, and is the recognition component of the classical complement activation pathway.

C1q binding studies were performed as previously described (Cragg et al., Blood 2004, 103:2738-2743). Briefly, Ramos B-cells in Iscoves media (#12440-053, Gibco/Invitrogen, Grand Island, N.Y.) with no serum were plated in 96-well V bottom plates at 5×10⁵/well in 100 μl. Cells were incubated with reagents for 15 minutes at 37° C., and normal human serum (NHS, #A113, Quidel Corp., San Diego, Calif.) diluted in Iscoves was then added at a volume of 50 μl to each well for a final concentration of 10, 5, 2.5, or 1.25% human serum. Fifty μl of media was added to the control well. For cobra venom factor (CVF) experiments, CVF was added to human serum complement samples at 20 Units CVF/mL of serum for 90 minutes at 37° C. prior to addition of serum to complement assays, and the dilution of serum by CVF accounted for when making sample dilutions.

The cells plus complement source were incubated for an additional 5 minutes at 37° C., and washed 2 times with cold PBS (#14040-133, Gibco/Invitrogen, Grand Island, N.Y.) via centrifugation and resuspended in 100 μl of PBS. Fifty μl from each well was transferred to a second plate for second step control staining. Both plates were stained for 15 minutes in the dark on ice with either FITC sheep anti-HU C1q (#C7850-06A, US Biological, Swampscott, Mass.) or FITC Sheep IgG (#11904-56P, US Biological, Swampscott, Mass.). Samples were washed, resuspended in cold PBS, and read immediately on a FACsCalibur flow cytometer and analyzed with Cell Quest software (Becton Dickinson, San Jose, Calif.).

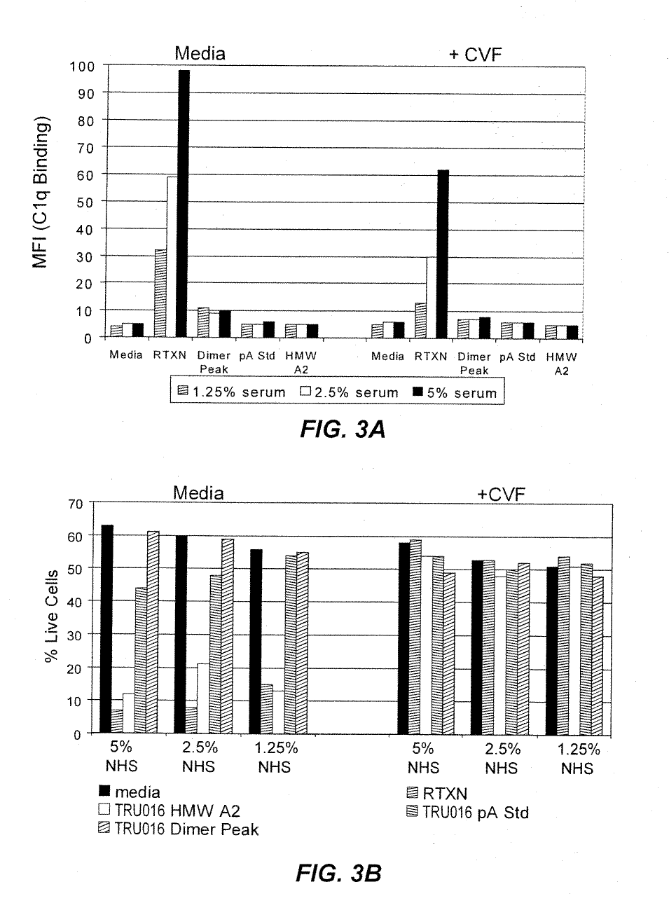

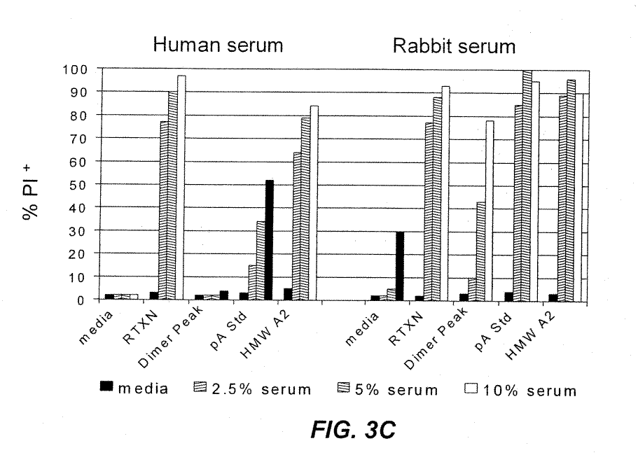

FITC C1q does not bind well to any subfractions of SEC purified TRU-016, although the higher molecular weight (HMW) or A2 aggregate fraction does show more binding than the other forms (FIG. 3A). In contrast, Rituxan showed a significant level of C1q binding, particularly at lower levels of NHS. The presence of CVF failed to completely block this binding, although the MFI levels are reduced significantly compared to media alone.

CDC assays were then performed to compare the ability of the different subfractions of the TRU-016 purified forms and Rituxan to mediate cell killing in the presence or absence of CVF and human serum complement (FIG. 3B). CDC assays were performed using propidium iodide staining to discriminate between live and dead cells after incubations of target cells with antibody, fusion proteins, ascites fluid, TRU-016 molecular forms, or media, and a source of complement such as human serum. Briefly, 3×10⁵ Ramos B-cells were pre-incubated with test reagents for 30-45 minutes at 37° C. prior to addition of complement. The prebound samples were centrifuged, washed, and resuspended in Iscoves with human serum (#A113, Quidel, San Diego, Calif.) at the indicated concentrations, then incubated for 90 minutes at 37° C. Samples were washed and propidium iodide (# P-1 6063, Molecular Probes, Eugene, Oreg.) was added to a final concentration of 0.5 μg/mL in PBS. The cells were incubated with the propidium iodide for 15 minutes at room temperature in the dark and then analyzed by flow cytometry on a FACsCalibur instrument with CellQuest software (Becton Dickinson).

Cell killing mediated by both the A2 fraction of TRU-016 and Rituxan was significantly reduced in the presence of CVF despite its failure to completely block C1q binding (FIG. 3B).

Human and rabbit complement were then compared for their CDC activity in the presence of the TRU-016. The CDC activity of TRU-016 molecular forms incubated with Ramos B cells and human or rabbit complement was measured (FIG. 3C). Ramos B cells were added to wells in serum free media. Rituxan or the dimer, HMW A2, or pA fractions of TRU-016 were added to cells to give a final concentration of 10 μg/ml, and incubated for 15 minutes at 37° C., prior to washing 1.5× in serum free media and addition of normal human serum (NHS) or rabbit complement (Pelfreez) at 10, 5, or 2.5%. Cells plus complement source were incubated 90 minutes at 37° C. Cells were washed once with cold PBS and propidium iodide (Molecular Probes #P3566) added to a final concentration of 0.5 μg/mL in cold PBS. Cells with P1 were incubated in the dark at RT for 15 minutes and analyzed by flow cytometry.

The origin of the complement fraction affects the CDC results obtained (FIG. 3C). Rabbit complement mediated higher levels of CDC than human complement in the presence of TRU-016 molecular forms. Interestingly, the dimer form of the TRU-016 mediated good CDC using rabbit complement, but very low CDC activity in the presence of human complement.

### Example 4

### Activity and Binding of TRU-016 Multimers

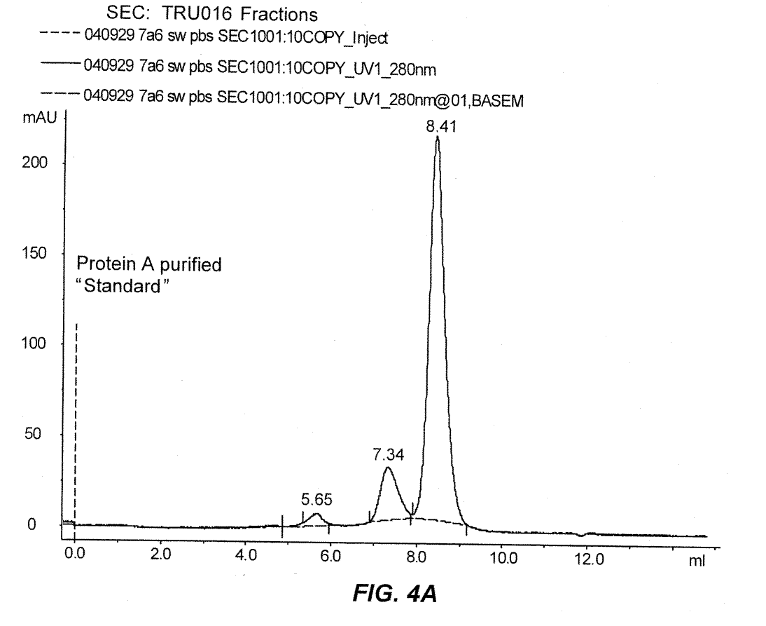

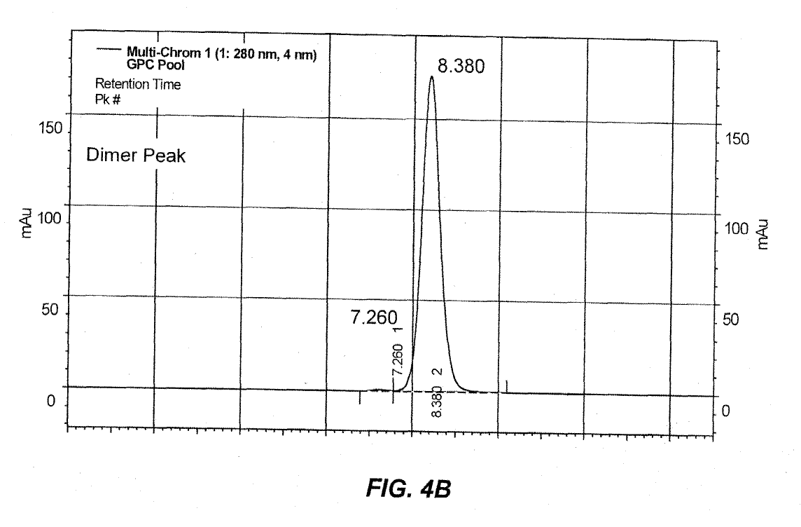

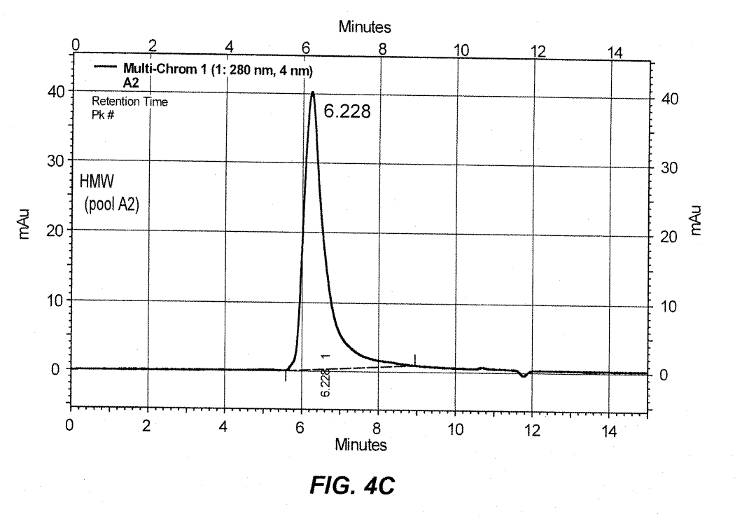

Experiments were performed to examine the biological activity of multimeric forms of TRU-016 (TRU-016 multimers) in solution. First, to determine the size of TRU-016 fusion protein in solution, protein A purified material was analyzed by SEC HPLC and revealed that TRU-016 exists in multiple forms in solution (FIG. 4).

HPLC size exclusion chromatography (SEC) traces were obtained from GPC purification of TRU-016, plotting absorbance versus retention time for the different fractions collected (FIG. 4). TRU-016 was purified from cell culture supernatants initially by affinity chromatography using Protein A Sepharose. The recombinant molecule was eluted from the column with 100 mM glycine, pH 3.5. 10 mL fractions of the eluted product were recovered and the eluted product was then brought to pH 5.0 with 20% of the eluted volume of 0.5 M 2-(N-Morpholino)ethanesulfonic acid (MES) pH6.0. The eluate was prepared for GPC purification by concentration of the sample to approximately 25 mg/mL TRU-016 and then filter sterilized in preparation for GPC purification. Size exclusion chromatography was performed on a GE Healthcare AKTA Explorer 100 Air apparatus, using a GE healthcare XK column and Superdex 200 preparative grade (GE Healthcare).

The HMW or A2 pools exhibited a retention time of approximately 6.23 minutes, while the most prominent form showed a retention time of 8.38 minutes. The reference standard used here (pA standard or std) is protein A purified material containing both dimers and HMW mulitimer forms, as shown in the first panel of FIG. 4. The most prominent form, migrating at a retention time of 8.38 minutes, most likely corresponds to the dimer molecule seen on non-reduced SDS-PAGE, and several minor forms most likely correspond to multimers that associate through non-covalent interactions as they are not evident on nonreducing SDS-PAGE. To separate these different forms of TRU-016, material obtained from protein A sepharose affinity chromatography of culture supernatants was further purified by GPC and HPLC fractionation to isolate the dimer form (identified as dimers” or “dimer peak”) from higher molecular weight multimers (identified as HMW or A2 agg fraction). Each of these three subfractions was then analyzed separately for functional activity in vitro using binding, ADCC, and CDC assays.

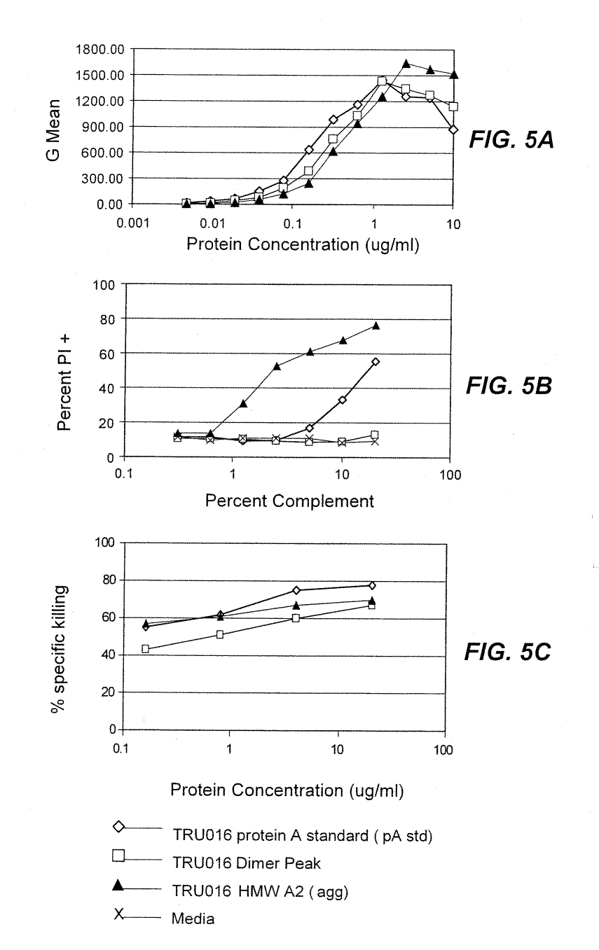

To explore whether the fractions isolated from SEC showed different binding properties, each fraction of TRU 016 SEC was tested for binding to Ramos cells. To determine the binding properties of SEC fractions, cells were suspended in staining media at a concentration of approximately 4×10⁶ cells/mL and then plated at 50 μl/well (2×10⁵ cells/well) in staining media. Serial dilutions of SEC fractions were then added to sequential wells, incubated for 45 minutes, washed, and binding activity was detected using FITC goat anti-human IgG. Samples were fixed in 200 μl 1% paraformaldehyde in PBS. Cells were analyzed by flow cytometry on a FACsCalibur instrument using Cell Quest software (BD Biosciences, San Jose, Calif.) (FIG. 5A).

To determine the CDC activity of SEC fractions, cells were suspended at 5×10⁵ cells/well in 75 μl IMDM. TRU 016 SEC fractions (75 μl) were added to the cells at twice the concentrations indicated. Binding reactions were allowed to proceed for 45 minutes prior to centrifugation and washing in serum free Iscoves. Cells were resuspended in Iscoves with human serum (#A113, Quidel, San Diego, Calif.) at the indicated concentrations. The cells were incubated 60 minutes at 37° C., washed, and resuspended in staining media with 0.5 μg/mL propidium iodide (PI, #P-16063, Molecular Probes, Eugene Oreg.). Samples were incubated 15 minutes at room temperature in the dark prior to analysis by flow cytometry using a FACsCalibur and CellQuest software (Becton Dickinson) (FIG. 5B).

To determine the ADCC activity of SEC fractions, BJAB, Ramos, and Daudi lymphoblastoid B cells (107) cells were labeled with 500 μCi/mL ⁵¹Cr sodium chromate for 2 hours at 37° C. in IMDM/10% FBS. PBMCs were isolated from heparinized, human whole blood by fractionation over Lymphocyte Separation Media (LSM, ICN Biomedical) gradients. Reagent samples were added to RPMI media with 10% FBS and five serial dilutions for each reagent were prepared. For combinations, the reagents were premixed and diluted prior to addition to the wells. The ⁵¹Cr labeled BJAB were added at (2×10⁴ cells/well). The PBMCs were then added at (5×10⁵ cells/well) for a final ratio of 25:1 effectors (PBMC):targets (BJAB). Reactions were set up in quadruplicate wells of a 96 well plate. TRU-016 SEC fractions were added to wells at a final concentration ranging from 10 ng/mL to 20 μg/mL as indicated on the graphs. Each data series plots a different SEC fraction at the titration ranges described. Reactions were allowed to proceed for 6 hours at 37° C. in 5% CO₂ prior to harvesting and counting. CPM released was measured on a Packard TopCounNXT from 50 μl dried culture supernatant. Percent specific killing was calculated by subtracting (cpm [mean of quadruplicate samples] of sample−cpm spontaneous release)/(cpm maximal release-cpm spontaneous release)×100 (FIG. 5C).

FIG. 5A shows the titration curves of the different SEC fractions binding to Ramos cells. All of the fractionated molecules bound to CD37 with similar binding curves except at the highest concentrations tested, where the HMW material exhibited better binding (higher fluorescence intensity) than the pA standard and the dimer peak forms.

Experiments were also performed to determine if the TRU 016 SEC fractions exhibited different levels of functional activity such as CDC and ADCC mediated target cell killing. The graph shown in FIG. 5B indicates that only the purified HMW multimer fraction mediated significant levels of CDC activity against Ramos B cells using human complement. The pA standard exhibited some CDC activity at higher concentrations, while the dimer peak form showed very little or no CDC activity at all concentrations tested.

ADCC assays were performed on serial dilutions of various TRU-016 size fractions using labeled BJAB B cells as targets and human PBMC as effector cells. TRU 016 SEC fractions were present in wells at a final concentration ranging from 10 ng/mL to 20 μg/mL as indicated in the graph shown in FIG. 5C. Each data series plotted a different SEC fraction at the titration ranges described. Data were plotted as % specific killing versus protein concentration. All of the SEC subfractions, including the pA standard, HMW or A2 fraction, and dimer peak, mediated potent, dose-dependent ADCC against BJAB target cells. Similar results were also obtained using Ramos cells as labeled targets (data not shown).

### Example 5

### Production of a CD20-specific Binding Molecule

CD20-specific SMIPs are described in co-owned US Patent Publications 2003/133939, 2003/0118592 and 2005/0136049. Production of an exemplary CD20-specific SMIP, TRU-015, is described below.

TRU-015 is a recombinant (murine/human) single chain protein that binds to the CD20 antigen. The binding domain was based on a publicly available human CD20 antibody sequence. The binding domain is connected to the effector domain, the CH2 and CH3 domains of human IgG1, through a modified CSS hinge region. TRU-015 exists as a dimer in solution and the dimer has a theoretical molecular weight of approximately 106,000 daltons. The nucleotide sequence encoding TRU-015 and the amino acid sequence of TRU-015 are respectively set out in SEQ ID NOs: 3 and 4.

Referring to the amino acid sequence set out in SEQ ID NO: 4, TRU-015 comprises the 2e12 leader peptide cloning sequence from amino acids 1-23; the 2H7 murine anti-human CD20 light chain variable region with a lysine to serine (VHL11S) amino acid substitution at residue 11 in the variable region, which is reflected at position 34; an asp-gly3-ser-(gly4ser)2 linker beginning at residue 129, with the linker having an additional serine at the end to incorporate the SacI restriction site for cassette shuffling; the 2H7 murine anti-human CD20 heavy chain variable region, which lacks a serine residue at the end of the heavy chain region, i.e., changed from VTVSS to VTVS; a human IgG1 Fc domain, including a modified hinge region comprising a (CSS) sequence, and wild type CH2 and CH3 domains.

The CHO cells that produce TRU-015 were cultured in a bioreactor using proprietary media. TRU-015 was purified using a series of chromatography and filtration steps including a virus reduction filter. The material was then concentrated and formulated with 20 mM sodium phosphate and 240 mM sucrose, with a resulting pH of 6.0. The composition is filtered before filling into glass vials at a concentration of 10 mg/mL. Each glass vial contained 5 mL of TRU-015 (50 mg/vial).

### Example 6

### Combinations of TRU-016 with TRU-015 or Rituxan Synergistically Increase Apoptosis in B Cells

Experiments examining the effect of B cell targeted SMIPS on B cell line apoptosis were performed. Each SMIP was tested individually and then in combination. Samples were analyzed at both 24 and 48 hours after initiation of incubation reactions. Annexin/PI Analysis was performed as follows: BJAB (courtesy of Ed Clark, University of Washington), Ramos (ATCC# CRL-1596), and Daudi cells were incubated 24 or 48 hours at 37° C. in 5% CO₂ in Iscoves (Gibco) complete media with 10% FBS at 3×10⁵ cells/mL and 20 μg/mL SMIP protein. In addition, 20 μg/mL goat anti-human IgG was added to reactions in order to cross link reagents on the cell surface. Cells were then stained with Annexin V-FITC and propidium iodide using the BD Pharmigen Apoptosis Detection Kit I (#556547), and processed according to kit instructions. Briefly, cells were washed twice with cold PBS and resuspended in “binding buffer” at 1×10⁶ cells/mL. One hundred microliters of the cells in binding buffer were then stained with 5 μL of Annexin V-FITC and 5 μL of propidium iodide. The cells were gently vortexed and incubated in the dark at room temperature for 15 minutes. Four hundred microliters of binding buffer was then added to each sample. They were then read and analyzed on a FACsCalibur (Becton Dickinson) instrument using Cell Quest software (Becton Dickinson).

Table 2 below shows that in the presence of crosslinking, treatment with TRU-016 had a more significant effect on apoptosis of cell lines than TRU-015 alone, although both molecules when used alone do induce some apoptosis. The increase varies depending on the cell line.

TABLE 2

| Bjab | Annexin V Positive |
|---|---|
| No SMIP | 17.5 |
| CD20 SMIP | 27 |
| CD37 SMIP | 30.6 |
| CD19 SMIP | 29.1 |
| CD20 + CD37 SMIP | 41 |
| CD20 + CD19 SMIP | 37.1 |
| CD37 + CD19 SMIP | 35.3 |

| | | plus GAM |
|---|---|---|
| Ramos | AnnexinV Positive | AnnexinV positive |
| cells alone | 3 | 3.3 |
| CD20 MAb | 1.4 | 3.1 |
| CD37 Mab | 18.3 | 8.7 |
| CD19 MAb | 3.7 | 3.1 |
| CD40 MAb | 3.9 | 8.3 |
| CD20 + CD37 | 32.3 | 35.7 |
| CD20 + CD19 | 5 | 10.5 |
| CD20 + CD40 | 5.7 | 19.4 |
| CD19 + CD37 | 26.9 | 50 |
| CD19 + CD40 | 8.2 | 18.4 |

### Example 7

### Combinations of TRU-016 with CD20-Specific Antibodies or SMIPs Synergistically Increase CDC

Experiments were performed to determine the CDC activity of combinations of TRU-016 with CD20-specific antibodies or SMIPS against B cells. The amount of reagents chosen for combination experiments was 0.5 μg/mL TRU-016 while that of TRU-015 was also 0.5 μg/ml. The concentration of rituxan was usually 0.04-0.06 μg/mL because of its higher activity in single reagent CDC experiments. In some experiments, the concentration of CD20 reagent was held constant at a suboptimal concentration, while the concentration of TRU-016 was varied to explore the minimal levels of CD37 directed reagent required to observe augmentation effects on CDC.

Cells were suspended in Iscoves (#12440-053, Gibco/Invitrogen, Grand Island, N.Y.) at 5×10E5 cells/well in 75 μl. TRU-016 (75 e-1), TRU-015, rituxan, or combinations of these reagents were added to the cells at twice the concentrations indicated. Binding reactions were allowed to proceed for 45 minutes prior to centrifugation and washing in serum free Iscoves. Cells were resuspended in Iscoves with human serum (#A113, Quidel, San Diego, Calif.) at the indicated concentrations. The cells were incubated 60 minutes at 37° C. Cells were washed by centrifugation and resuspended in 125 μl PBS with 2% FBS (#16140-071, Gibco, Invitrogen, Grand Island, N.Y.), staining media. The cells were transferred to FACS cluster tubes (#4410, CoStar, Corning, N.Y.) and 125 μl staining media with 5 μl propidium iodide (PI, #P-16063, Molecular Probes, Eugene Oreg.) was added. Samples were incubated 15 minutes at room temperature in the dark prior to analysis by flow cytometry using a FACsCalibur and CellQuest software (Becton Dickinson).

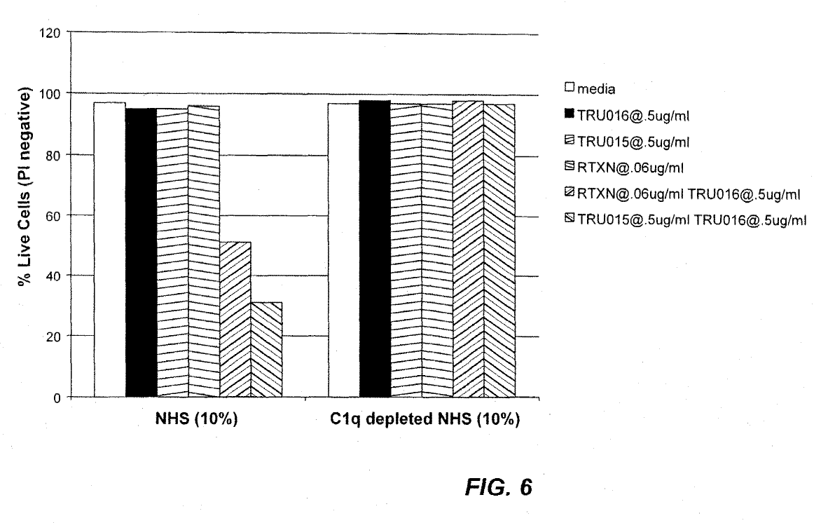

FIG. 6 shows that at suboptimal concentrations for killing as a single agent, TRU-015 and rituxan exhibit high levels of CDC activity when combined with TRU-016. TRU-016 alone fails to mediate CDC unless aggregates are present. Depletion of C1q from the reactions results in the elimination of all CDC activity observed.

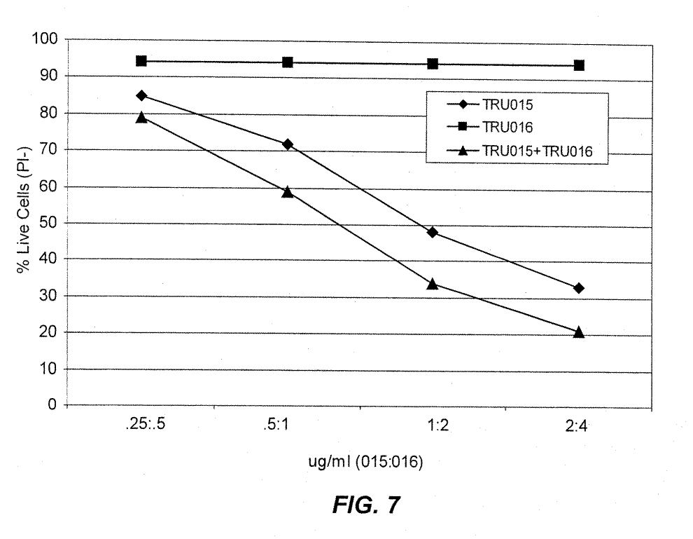

FIG. 7 shows a combination experiment performed on DHL-4 B cells. Addition of TRU-016 to the CDC reactions results in a downward shift to the TRU-015 killing curve, demonstrating more effective killing at each concentration tested even though TRU-016 exhibits little or no activity alone.

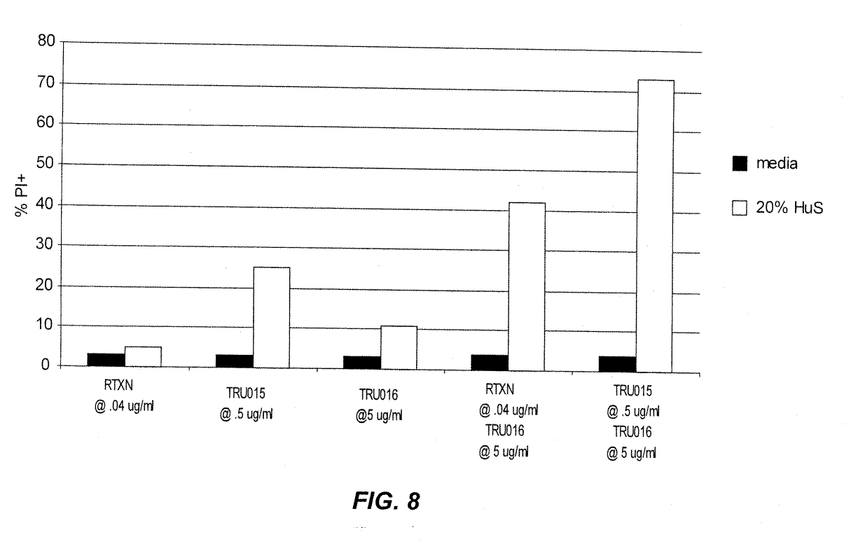

FIG. 8 shows another CDC experiment where the sample reagents were mixed at the following ratios: 0.5 μ/mL TRU-015, 0.5 μg/mL TRU-016, and 0.06 μg/mL rituxan. Again, the single agents are used at suboptimal concentrations in order to see augmentation effects in the presence of TRU-016. For both TRU-015 and rituxan, TRU-016 enhances the level of CDC killing when added to the assays.

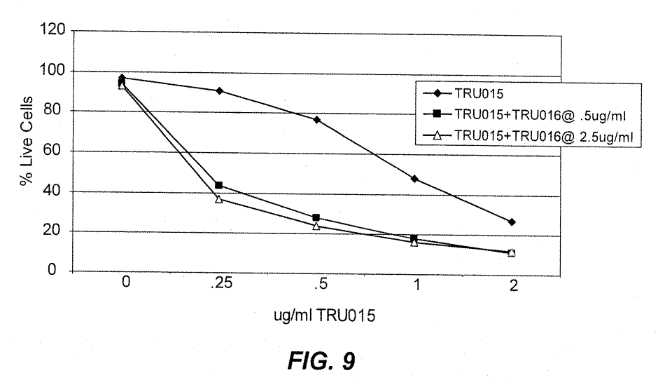

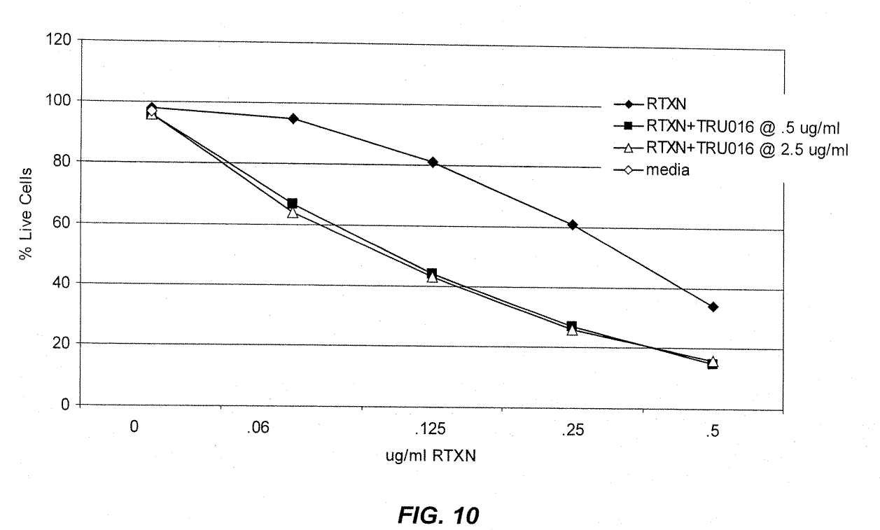

FIGS. 9 and 10 show graphical representations of the data for CDC assays where the concentration of TRU-015 or rituxan was kept constant and TRU-016 concentration was increased. Again, CDC activity was greater when TRU-016 was added to the reactions, but increasing the concentration of TRU-016 to 2.5 μg/mL from 0.5 μg/mL did not significantly increase the CDC-mediated killing in these experiments.

### Example 8

### TRU-016 Augments the ADCC and the CDC Activity of CD20-Specific Antibodies and SMIPs

Experiments were performed to determine if combinations of TRU-016 SMIP with CD20-specific antibodies or SMIPs could augment ADCC and CDC activity against B cell targets.

BJAB, Ramos, and Daudi lymphoblastoid B cells (10E7) cells were labeled with 500 μCi/mL ⁵¹Cr sodium chromate for 2 hours at 37° C. in IMDM/10% FBS. The labeled BJAB cells were washed three times in RPMI/10% FBS and resuspended at 4×10E5 cells/mL in RPMI. Heparinized, human whole blood was obtained from anonymous, in-house donors and PBMC isolated by fractionation over Lymphocyte Separation Media (LSM, ICN Biomedical) gradients. Buffy coats were harvested and washed twice in RPMI/10% FBS prior to resuspension in RPMI/10% FBS at a final concentration of 3×10E6 cells/ml. Cells were counted by trypan blue exclusion using a hemacytometer prior to use in subsequent assays. Reagent samples were added to RPMI media with 10% FBS at 4 times the final concentration and five serial dilutions for each reagent were prepared. For combinations, the reagents were premixed and diluted prior to addition to the wells. These reagents were then added to 96 well U bottom plates at 50 μl/well for the indicated final concentrations. The ⁵¹Cr labeled BJAB were added to the plates at 50 μl/well (2×10E4 cells/well). The PBMCs were then added to the plates at 100 μl/well (3×10E5 cells/well) for a final ratio of 15:1 effectors (PBMC):target (BJAB).

Effectors and targets were added to media alone to measure background killing. The ⁵¹Cr labeled BJAB were added to media alone to measure spontaneous release of ⁵¹Cr and to media with 5% NP40 (#28324, Pierce, Rockford, Ill.) to measure maximal release of 51 Cr. Reactions were set up in quadruplicate wells of a 96-well plate. SMIPs were added to wells at a final concentration ranging from 12 ng/mL to 10 μg/mL as indicated on the graphs. For SMIP combinations, the reagents were mixed prior to addition to the wells. Each data series plots a different single SMIP or combination at the titration ranges described. Reactions were allowed to proceed for 6 hours at 37° C. in 5% CO₂ prior to harvesting and counting. Fifty μl of the supernatant from each well was then transferred to a Luma Plate 96 (#6006633, Perkin Elmer, Boston, Mass.) and dried overnight at room temperature. CPM released was measured on a Packard TopCounNXT. Percent specific killing was calculated by subtracting (cpm {mean of quadruplicate samples} of sample−cpm spontaneous release)/(cpm maximal release-cpm spontaneous release)×100.

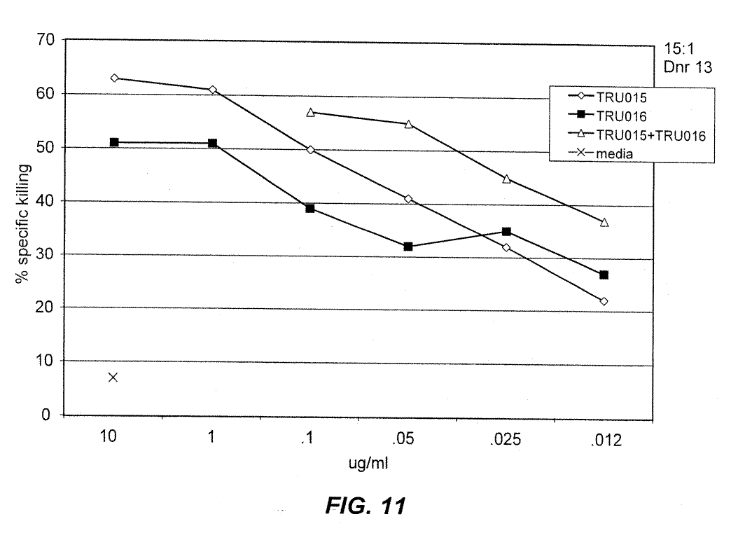

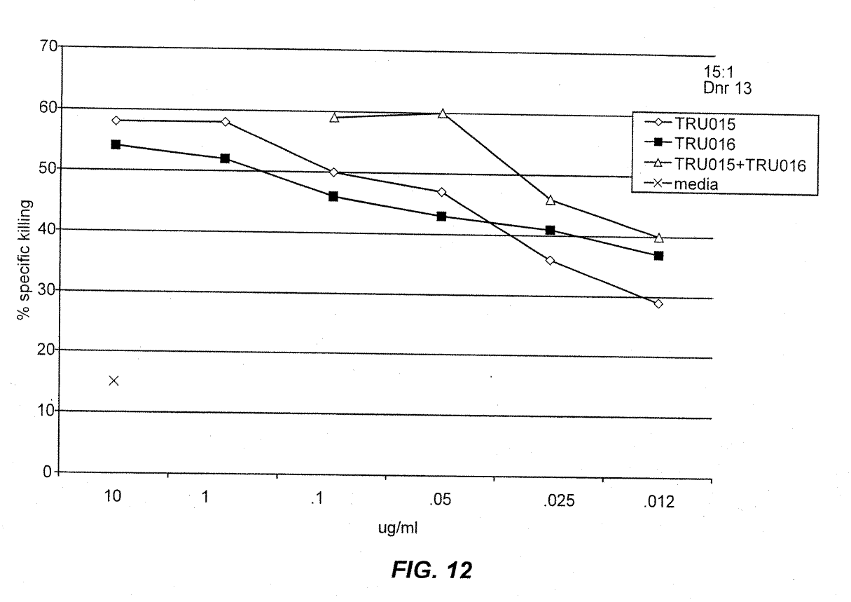

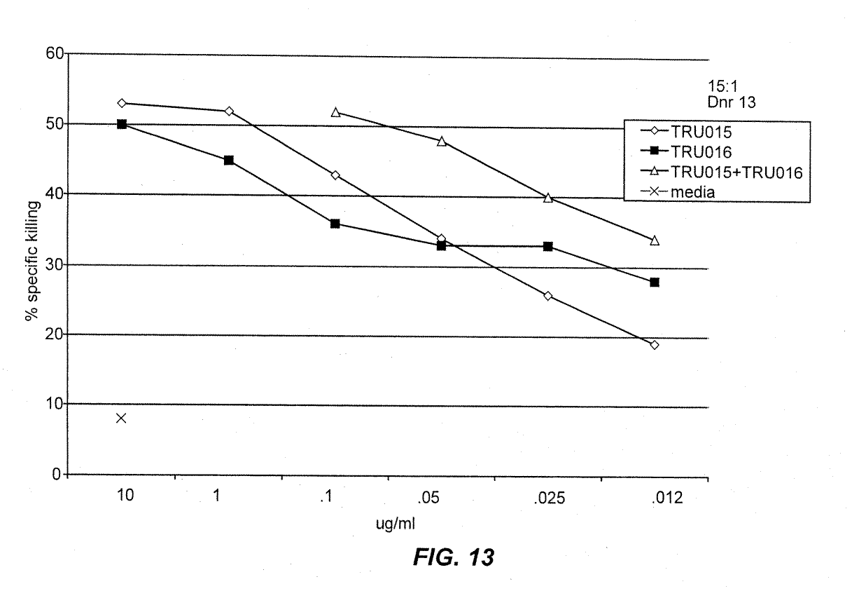

Data were plotted as % specific killing versus SMIP concentration. The effector to target ratio is indicated on each figure, and the target cell line was also indicated. FIGS. 11, 12, and 13 show data for experiments on different cell lines (BJAB, Daudi, and Ramos) where the same donor was used.

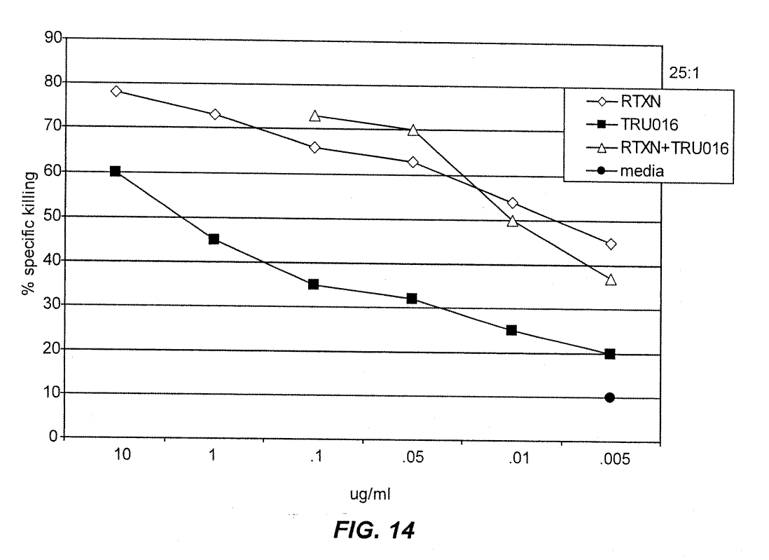

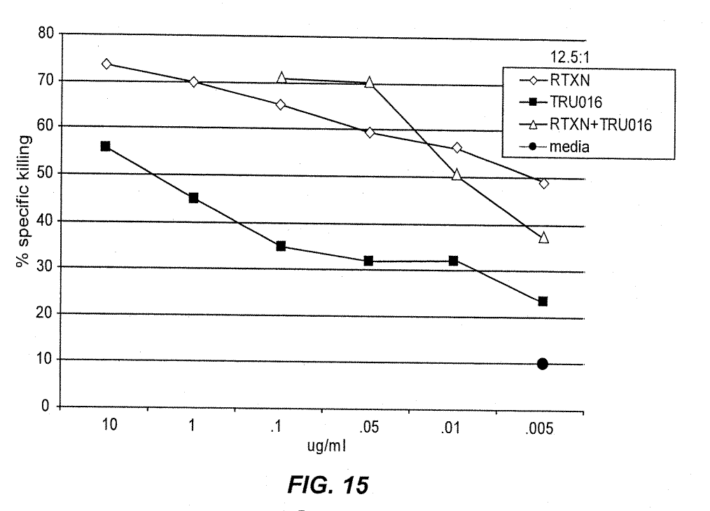

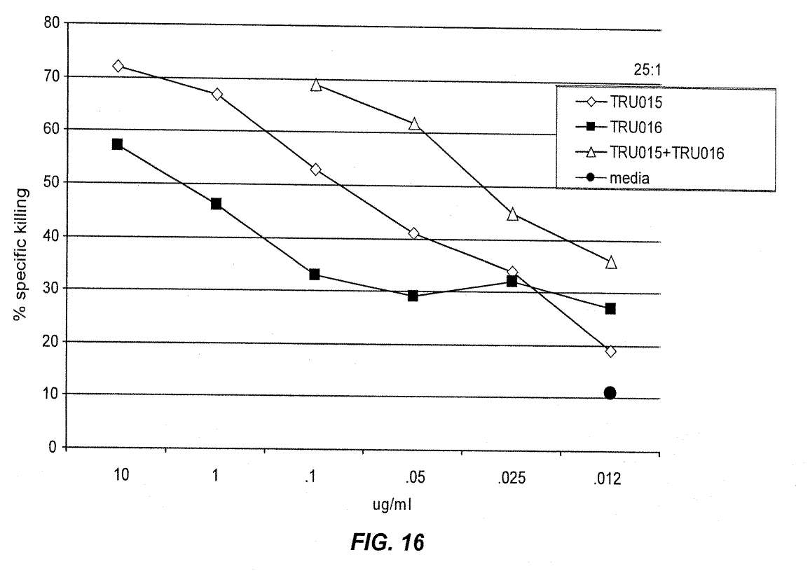

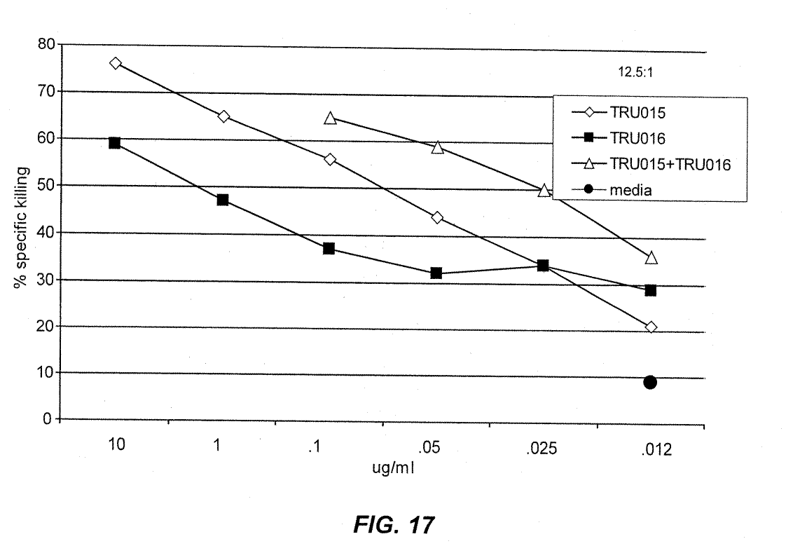

In FIGS. 14 and 15 (rituxan+TRU-016) and FIGS. 16 and 17 (TRU-015+TRU-016) data is presented for experiments in which the target cell line used was BJAB. The specific killing observed for each combination was greater than either single reagent alone at the same concentration, indicating that the CD20 and CD37 targeted SMIPs augment the killing mediated by the other, although the augmentation effect is not completely additive.

Thus, TRU-016 can enhance CD20-specific SMIP or CD20-specific antibody ADCC mediated killing of B cells.

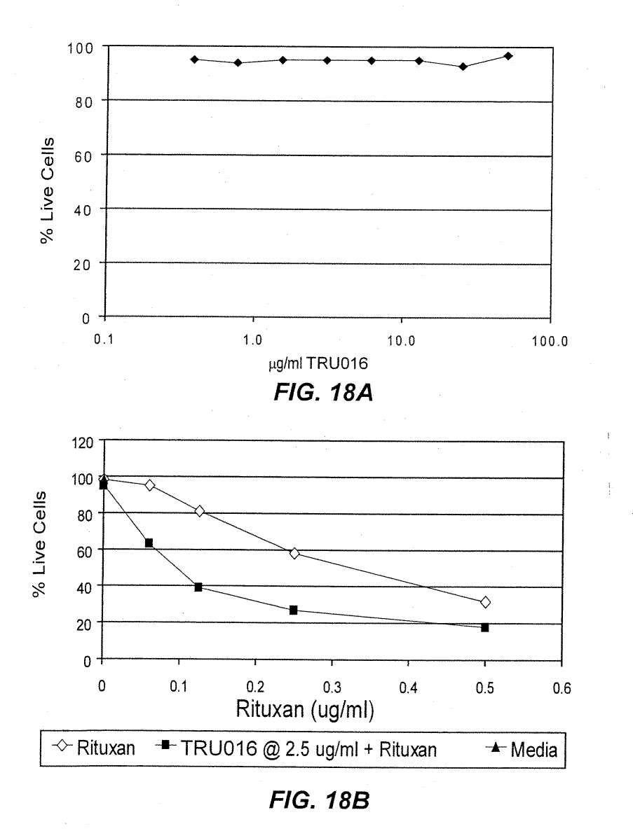

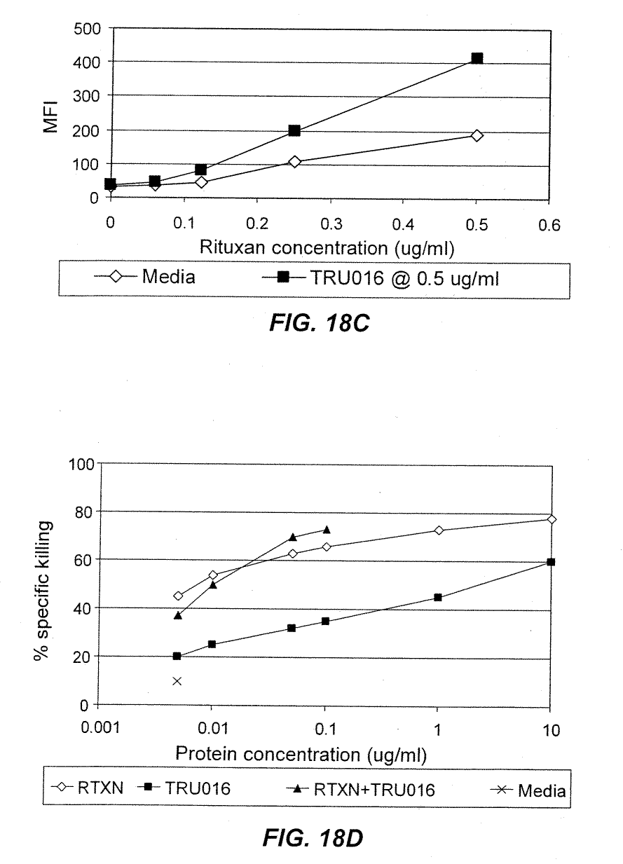

Initial experiments to explore the effects of combinations of TRU-016 with CD20-directed antibodies were designed to determine the relative amounts of each reagent to use so that CDC synergy could be detectable. Ramos cells were suspended in IMDM, and TRU-016, Rituxan, or combinations of these reagents were added to the cells to the final concentrations indicated in FIG. 18. Binding reactions were allowed to proceed for 45 minutes prior to centrifugation and washing in serum free Iscoves. Cells were resuspended in Iscoves with 10% NHS. The cells were incubated 60 minutes at 37° C. In experiments shown in FIG. 18A-C, cells were washed by centrifugation and resuspended in staining media containing 0.5 μg/mL propidium iodide (PI, #P-16063, Molecular Probes, Eugene Oreg.). Samples were incubated 15 minutes at room temperature in the dark prior to analysis by flow cytometry using a FACsCalibur and CellQuest software (Becton Dickinson).

The more highly purified TRU-016 dimer peak is a poor mediator of CDC when used alone, as shown in FIG. 18A by the flat dose-response curve even at high concentrations. Because CD20 directed reagents were efficient inducers of CDC activity, non saturating amounts of the CD20 directed reagents were desirable in combination experiments, so that synergy between the reagents could be detected. From these initial studies, the usual amount of reagent chosen for combination experiments was 0.5 μg/mL or 2 μg/mL TRU-016. The concentration of Rituxan was usually 0.04-0.06 μg/mL because of its higher activity in single reagent CDC experiments. In some experiments, the concentration of CD20 reagent was held constant at a suboptimal concentration, while the concentration of TRU 016 was varied to explore the minimal levels of CD37 directed reagent required to observe augmentation effects on CDC. Thus, TRU-016 alone fails to mediate CDC unless aggregates are present.

FIG. 18B shows a graph of the percentage of live cells (PI negative) observed over the titration range indicated (0.06-0.5 μg/ml) when Rituxan is used alone or in combination with TRU-016 at 2.5 μg/ml. Rituxan, when used at a range of suboptimal doses for killing as a single agent, exhibits higher levels of CDC activity at each concentration when combined with TRU-016 (FIG. 18B). Depletion of C1q from the reactions results in the elimination of all CDC activity observed (FIG. 3B).

In FIG. 18C, samples were also incubated with FITC anti-C1q for 45 minutes on ice prior to analysis by flow cytometry. Lymphocyte gating was on compromised cells. The percentage of cells in this gate increased with increasing Rituxan concentration, and the relative MFI for this population of cells was graphed. FIG. 18C shows the results of a CDC experiment where the sample reagents were mixed at the following ratios: 0.5 μg/mL for TRU-016, and Rituxan concentrations ranging from 0.06 μg/mL to 0.5 μg/mL, and cells stained with PI prior to flow cytometry. The results show a dose dependent increase in MFI with increasing doses of Rituxan. The addition of TRU-016 dimer forms resulted in an additional increase in the MFI at each concentration of Rituxan. A similar series of CDC assays were performed, keeping the concentration of Rituxan constant and increasing the TRU-016 concentration. Again, CDC activity was greater when TRU-016 was added to the Rituxan reactions, but increasing the concentration of TRU-016 to 2.5 μg/mL from 0.5 μg/mL did not significantly increase the CDC mediated killing in these experiments (data not shown).

Rituxan and TRU-016 proteins used alone and in combination with one another were compared for their ADCC activity in vitro using a similar concentration range as that used for the CDC assays. FIG. 18D shows the results of an ADCC assay with labeled Ramos cell targets and human PBMC effector cells at an effector to target ratio of 25:1, using TRU-016 or Rituxan, alone and in combination with one another over the concentration ranges indicated. Similar data were obtained at an effector:target ratio of 12.5:1. Both the TRU-016 dimer form and Rituxan mediate significant levels of ADCC against Ramos cells expressing the CD20 and CD37 target antigens; however, the combination of the two reagents does not result in significant augmentation in the level of killing.

### Example 9

### TRU-016 Induces Apoptosis in B Cells

Experiments examining the effect of TRU-016 on B cell line apoptosis were performed. Initial assays of the effects on apoptosis of TRU-016 molecules targeted to different B cell receptors were performed using protein A purified material that still contained higher order aggregates. After 24 hour treatment with CD37 antibodies or engineered TRU-016 molecules, similar patterns of increased apoptosis were observed in multiple experiments using annexin V positive cell percentages as a measure of apoptotic activity and both Ramos and BJAB cells as binding targets (data not shown).

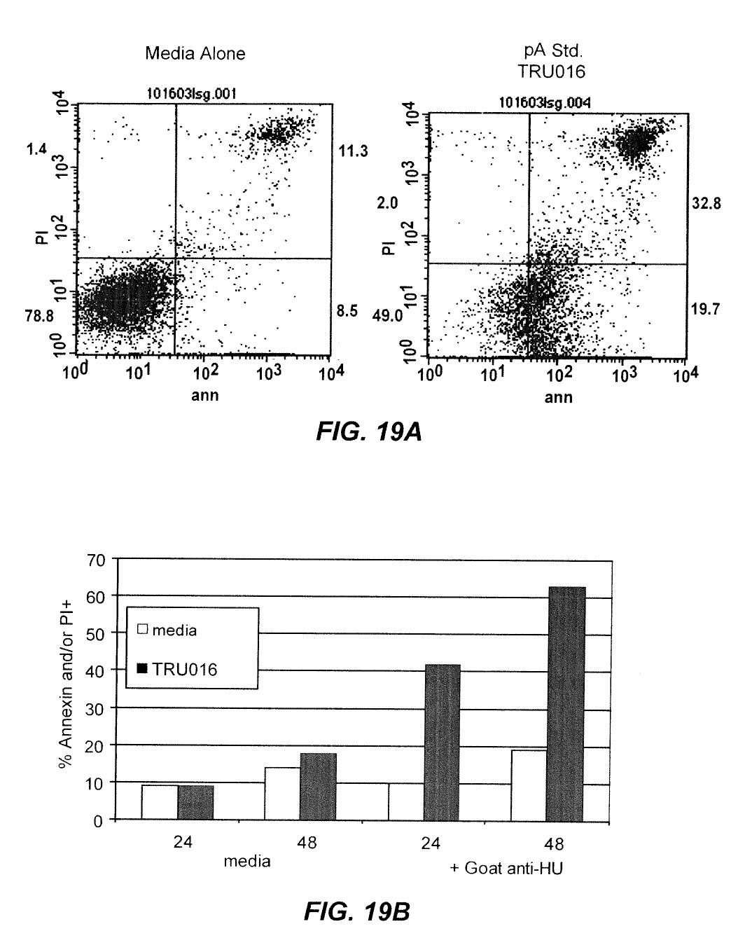

FIG. 19A demonstrate that apoptosis is significantly increased after incubation of B cell lines with unfractionated TRU-016. FIG. 19A shows a dot plot of Annexin V-PI staining of Ramos cells after incubation for 24 hours with the TRU-016 (10 g/mL). The % of annexin V-PI double positive cells increased from 11.3% of the total population to 32.8%, and the % of annexin V positive-PI negative cells increased from 8.5% to 19.7%, indicating that apoptosis is induced after exposure to TRU-016. Similar data were obtained whether Ramos or BJAB cells were used as the binding targets in these assays.

Further experiments examining the effect of TRU-016 on B cell line apoptosis were performed using the more highly purified dimer form of TRU-016 (FIG. 19B). Samples were analyzed at both 24 and 48 hours after initiation of incubation reactions. Annexin/PI analysis was performed on several cell types using 20 μg/mL TRU-016 protein. Because apoptosis was reduced using the dimer form of TRU-016, 20 μg/mL goat anti-human IgG was added to reactions in order to cross link reagents on the cell surface. Cells were then stained with Annexin V-FITC and propidium iodide. The data shown in FIG. 19B demonstrates that the TRU-016 dimer peak induces apoptosis of Daudi cells after 24-48 hours, but that the presence of a crosslinking agent such as anti-human IgG results in a significant increase in the level of CD37 targeted apoptosis.

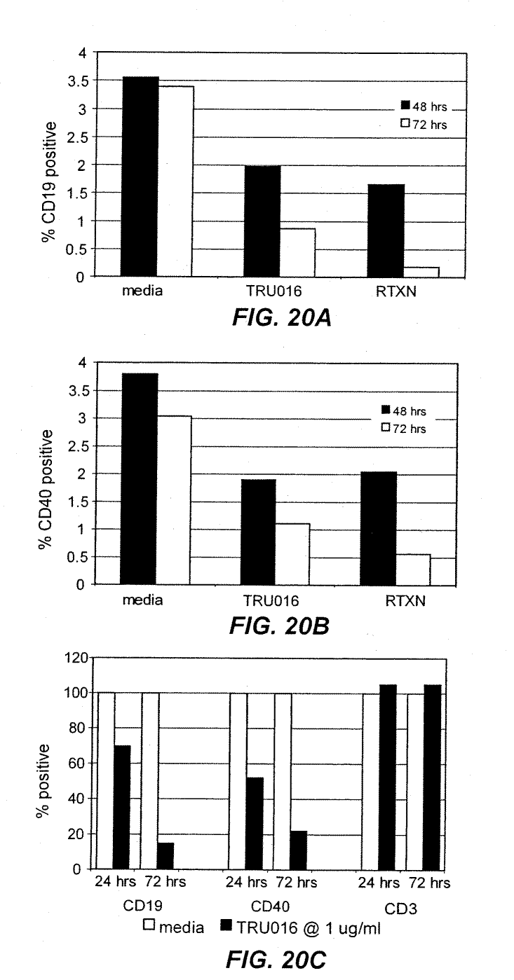

Experiments were also performed to determine the effect of TRU-016 on normal human B cells in culture using human PBMCs. FIGS. 20A and 20B shows results from one such experiment, with columnar graphs of the percentage of CD1 g or CD40 positive lymphocytes (B cells) present in PBMC cultures treated for 48-72 hours with media alone, TRU-016, or Rituxan.

Human PBMCs were isolated from whole blood by LSM density centrifugation. Cells were incubated for 48 or 72 hours with 1 μg/mL of Rituxan or TRU-016. A portion of the incubation reaction was harvested at 48 hours and again at 72 hours after initiation of the experiment. PBMCs were washed and incubated with FITC anti-CD19, FITC anti-CD40, or FITC anti-CD3 for 45 minutes on ice. The percentage of total lymphocytes staining with these reagents was then tabulated and compared to PBMC samples incubated under similar conditions but without test reagents, and stained as for the treated samples. FIGS. 20A and B show columnar graphs of the fraction of the total lymphocyte population (%) which give a positive FACs signal after 48 and 72 hours with the indicated reagents. FIG. 20C shows a composite graph from a similar experiment, showing the percent reduction from the original number of lymphocytes expressing the indicated CD antigen (i.e. CD19, CD40 or CD3 positive) after incubation of PBMCs with TRU-016 (at 1 μg/ml) for 24 and 72 hours.

In the presence of crosslinking, treatment with the TRU-016 dimer form or Rituxan resulted in a reduction in the percentage of B lymphocytes in PBMC cultures as measured by positive staining for CD19 and CD40. Although the percentage of B lymphocytes in culture was low at the outset of the experiment, coculture with Rituxan or TRU-016 decreased the number of CD19 and CD40 positive lymphocytes in the PBMC culture by approximately 1.5-2 fold after 48 hours, and by more than 3 fold after 72 hours. This general pattern of B cell depletion after 48-72 hours was reproducible in all normal PBMC cultures tested, regardless of the initial starting percentage of B lymphocytes in these cultures, which ranged from approximately 3% to as much as 7% of the total lymphocytes, depending on the sample.

FIG. 20C shows a columnar graph of the percentage depletion of B lymphocytes compared to T lymphocytes in short term PBMC cultures incubated with TRU-016 for 24 to 72 hours. These data indicate that the TRU-016 is capable of specific depletion of CD37 positive B lymphocytes from normal peripheral blood cultures, and that the low level of binding by TRU-016 to non-B lymphocytes (FIG. 1C) is insufficient to mediate significant depletion of these lymphocytes from the cell population.

### Example 10

### Combinations of TRU-016 and Rituximab Synergistically Reduce Tumor Volume in a Murine Tumor Xenograft Model

Mouse tumor xenograft studies exploring combination therapies were performed using nude mice (Harlan) and Ramos or Daudi human tumor lines. Ramos or Daudi tumor cells were grown in T 50 flasks in IMDM/10% FBS until they reached 80% confluency. Five million (5×10⁶) cells were used as a tumor inoculum per mouse. Cells were injected subcutaneously in the right flank using PBS in a total volume of 0.1 mL or 5.0×10⁷/mL. Nude mice were allowed to develop tumors and sorted into groups based on tumor size/volume. For each treatment group, 12 mice with a mean tumor volume of approximately 222 mm³ (range=152-296 mm³) were used. Some mean tumor volumes ranging from 237-251 mm³ were also used. Animals were injected intravenously (IV) at days 0, 2, 4, 6, and 8 with one of the following reagents: TRU-016 GPC POI (peak of interest), 200 μg/mouse; rituxan, 200 μg/mouse, or human IgG (control) at 200 or 400 μg/mouse as single reagents, or as the following combinations of reagents: Rituxan+TRU-016 at 100 μg each per mouse; or Rituxan+TRU-016 at 200 μg each per mouse. Tumor volume was measured daily with calipers until completion of the experiment (sacrifice or regression). Tumor volume as a function of treatment time was plotted for each animal and results were also averaged within each group.

Similar studies were also performed using smaller tumors, with mice sorted into groups with smaller mean tumor volume ranging between 153-158 mm³, and with larger tumors but using Daudi cells rather than Ramos cells. These studies were performed in an AAALAC accredited animal facility and animal use program in accordance with guidelines from an Institutional Animal Care and Use Committee (IACUC).

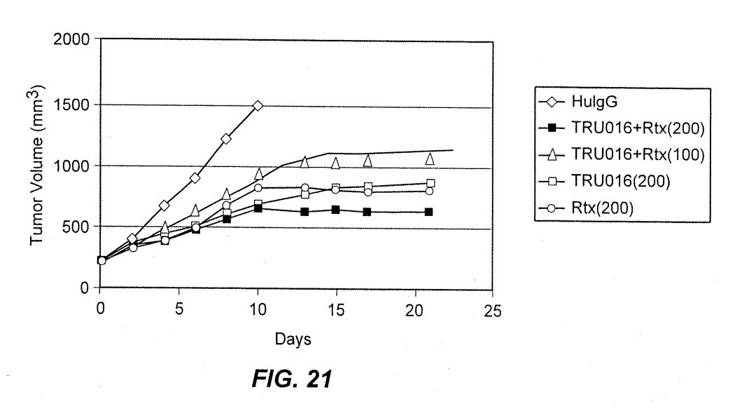

FIG. 21 graphs the efficacy of TRU-016 compared to huIgG, rituxan, and the combinations at 100 μg and 200 μg each averaged over each group of 12 animals. Tumor volume was plotted as a function of time after treatment with the IV injection(s). The average tumor volume after treatment with TRU-016 was smaller than that observed using the negative control (huIgG). When % survival or % tumor free animals were graphed, the higher dose combination therapy exhibited higher anti-tumor activity in this in vivo tumor model. However, at the lower dose (100 μg each), the combination therapy was not as effective as each single reagent at a higher dose.

These data indicate that TRU-016 therapy, when used in combination with rituxan at the appropriate doses, will have greater efficacy in treating patient tumors than rituxan therapy alone.

### Example 11

### TRU-016 Reduces Tumor Volume and Increases Survival in a Murine Tumor Xenograft Model

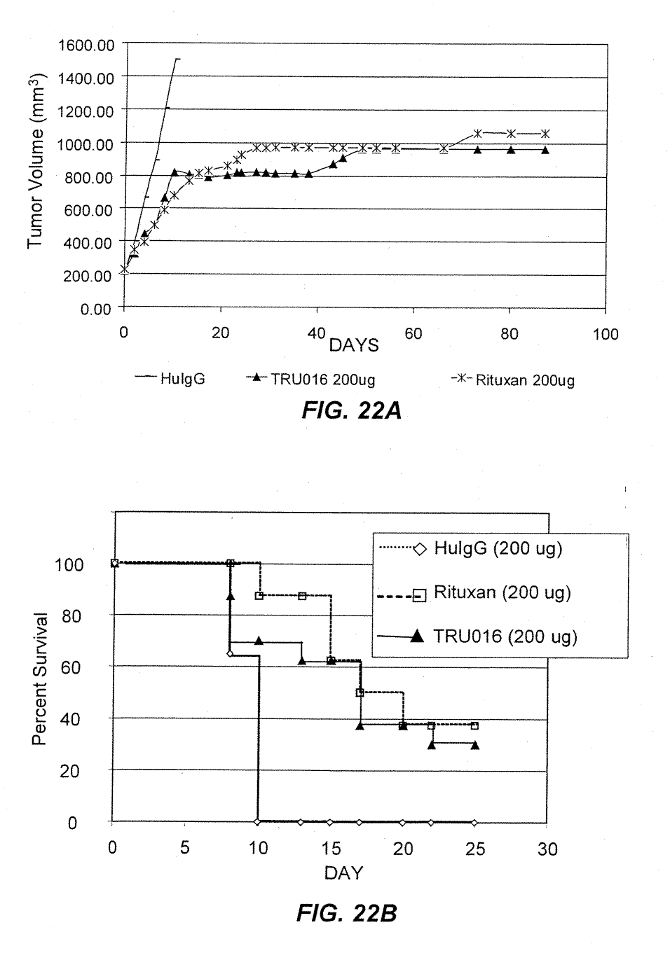

Mouse tumor xenograft studies were performed using nude mice (Harlan) and Ramos or Daudi human tumor lines. Three different studies were performed based on tumor type and tumor size at the time of treatment with the TRU-016 or other test reagent. Ramos or Daudi tumor cells were grown and (5×10⁶) cells were injected subcutaneously in the right flank to inoculate each treated mouse with the tumor. Nude mice were allowed to develop tumors and sorted into groups based on tumor size/volume. In the first study, for each treatment group, 12 mice with a mean tumor volume of 155-237 mm³ were used. Animals were injected intravenously (IV) at days 0, 2, 4, 6, and 8 with one of the following reagents: Rituximab, 200 μg/mouse; TRU-016 GPC dimer peak, 200 μg/mouse; or human IgG (control), 400 μg/mouse. Tumor volume was measured daily with calipers until completion of the experiment (sacrifice or regression). Tumor volume as a function of treatment time was plotted for each animal and results were also averaged within each group. Group averages were shown in FIG. 22A, while FIG. 22B shows a comparison of the percent survival data for each group of mice as a function of time.

FIG. 22A shows the efficacy of TRU-016 compared to huIgG and Rituxan in the Ramos tumor model, averaged over each group of 12 animals. Tumor volume was plotted as a function of time after treatment with the IV injection(s). The average tumor volume after treatment with the TRU-016 was smaller than that observed using the negative control (huIgG). FIG. 22B graphs the survival curves for the different treatment groups, comparing huIgG, Rituxan, and TRU-016. Administration of TRU-016, utilizing the more demanding Ramos tumor model with increased baseline tumor volume, resulted in an inhibition of tumor growth rate relative to human IgG (data not shown). Administration of TRU-016 to mice with the smaller Ramos tumors resulted in both an inhibition of tumor growth and increased median survival times.

### Example 12

### TRU-016 Does Not Affect the CDC Activity of Other B Cell Surface Receptors

To determine whether the TRU-016 molecule augments the level of CDC activity resulting from treatment with antibodies to other B cell surface receptors, in addition to CD20, such as MHCII, CD19, CD80/86, and CD40, a panel of experiments was performed similar to those just described for CD20-CD37 directed combinations.

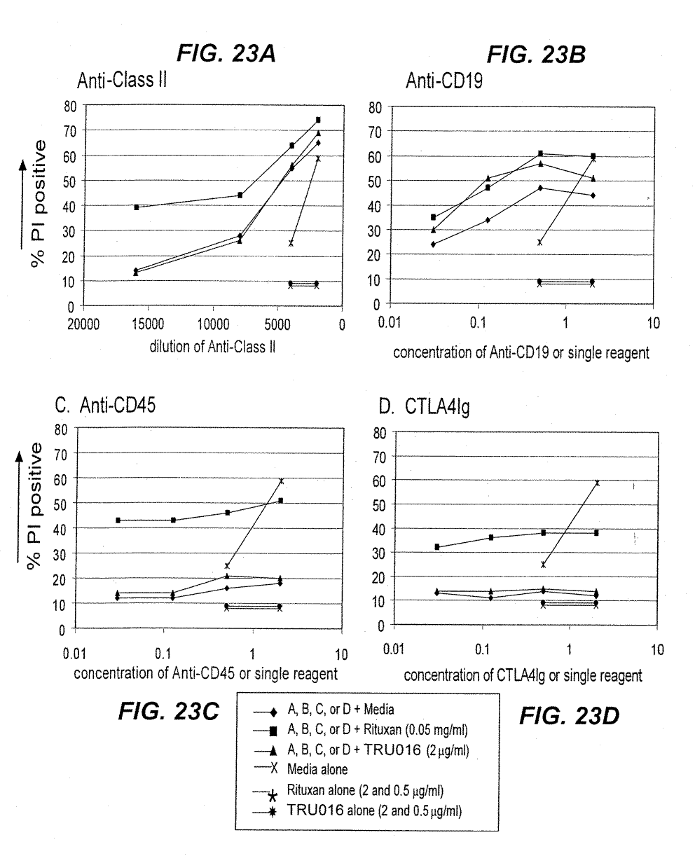

Ramos cells were added to wells in Iscoves complete media with 10% FBS. The MAbs (reagent B: HD37-anti CD19, reagent C, 9.4-anti-CD45), fusion protein (reagent D: CTLA-4 muIg-IgG2a, Ancell #501-820), and ascites fluid (reagent A: HB10a-anti-MHCII), were added at the indicated dilutions (see FIG. 23) and duplicate reactions were set up with and without Rituximab (at 0.05 μg/ml) or TRU-016 (at 2 μg/ml) added. Reactions were incubated for 30 minutes at 37° C. The cells were washed and NHS was added to a final concentration of 10% in serum free media. Cells were incubated for 90 minutes at 37° C. with the complement source. The cells were washed; propidium iodide was added to a final concentration of 0.5 μg/mL in PBS; the cells were incubated in the dark at room temperature for 15 minutes; and then cells were assayed by flow cytometry. Each graph in panels A-D of FIG. 23 plots the % PI positive cells over the titration ranges indicated.

In general, the data indicate that there was not a significant difference in the level of CDC activity when antibodies directed to these receptors were used alone or in combination with the TRU-016 (FIG. 23A-D). There may be a slight increase in CDC levels for the CD1 g and CD45 directed reagents when used with TRU-016 at suboptimal concentrations. However, the differences in CDC levels are not nearly as significant as those observed for the CD20-CD37 combination. In addition to the augmentation of CDC when CD20 and CD37 directed reagents are used in combination, there appears to be augmentation in the level of killing observed using combinations of anti-classII (HB10a), anti-CD1 g, anti-CD45 (9.4) or CTLA4Ig with Rituxan at the suboptimal dose.

### Example 13

### TRU-016 Does Not Augment the CDC Activity of Other Targeted Receptors, Including MHCII, CD19, CD80/86, and CD40

To determine whether the TRU-016 molecule augments the level of CDC activity resulting from treatment with antibodies to other B cell surface receptors, in addition to CD20, a panel of experiments was performed similar to those described for CD20-CD37 directed combinations (see Example 8). The results of these experiments are shown in FIG. 23. In general, there was not a significant difference in the level of CDC activity when antibodies directed to these receptors were used alone or in combination with the TRU-016. CDC levels slightly increased in response to CD19 and CD45 directed reagents when used with TRU-016 at suboptimal concentrations. However, the differences in CDC levels were not nearly as significant as those observed for the CD20-CD37 combination (see Example 8). In addition to the augmentation of CDC when CD20 and CD37 directed reagents are used in combination, there appeared to be augmentation in the level of killing observed using combinations of anti-MHCII (HB10a), anti-CD1 g, anti-CD45 (9.4) or CTLA4Ig with Rituxan at the suboptimal dose.

### Example 14

### TRU-016 Increases Survival in a Murine Tumor Xenograft Model

Mouse tumor xenograft studies beyond those described in Example 11 were performed to examine the efficacy of TRU-016 in increasing long-term survival using nude mice (Harlan) and either Ramos or Daudi human tumor cell lines.

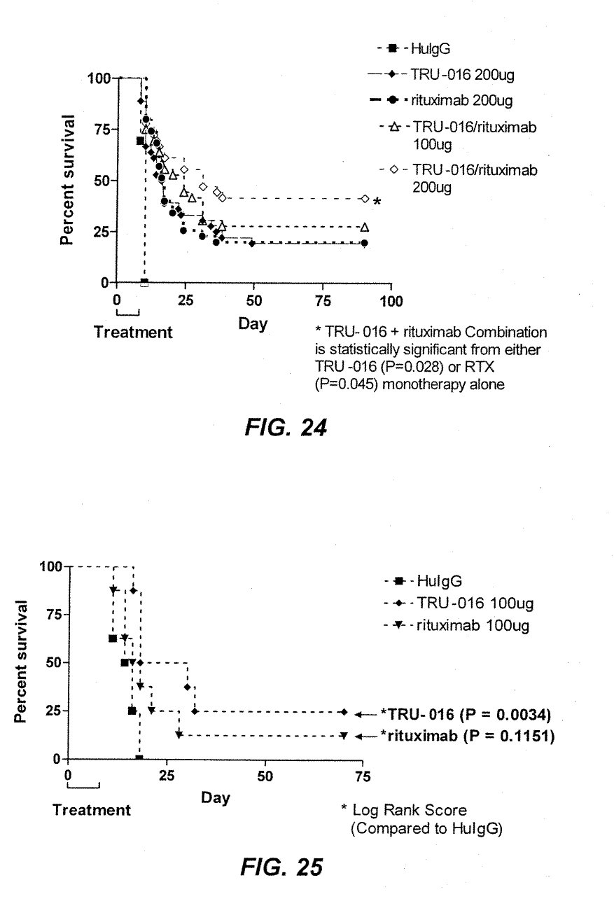

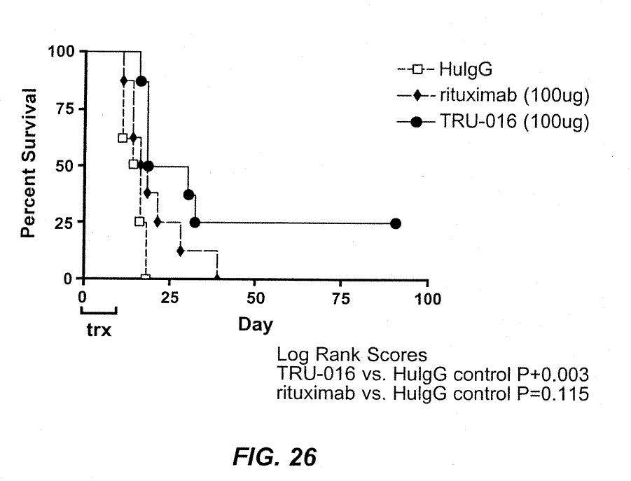

Ramos and Daudi tumor cells were separately grown and (5×10⁶) cells were injected subcutaneously in the right flank of mice to initiate the formation of mouse tumor xenografts. After tumor development, mice were sorted into groups based on tumor size/volume (day 0). Animals were injected intravenously (IV) at days 0, 2, 4, 6, and 8 with one of the following reagents: rituximab, 200 μg/mouse; TRU-016, 200 μg/mouse; rituximab+TRU-016 at 100 or 200 μg/mouse; or human IgG (control), 400 μg/mouse. Tumor volume was blindly measured three times weekly with calipers until completion of the experiment (sacrifice or regression). Tumor volume as a function of treatment time was plotted for each animal and results were averaged within each group. FIG. 24 shows the percent survival of mice with Ramos tumors (up to 90 days) after treatment with TRU-016, rituximab, or a combination thereof. The combination treatment with TRU-016+rituximab significantly increased median survival time versus treatment with single agent therapy alone. FIGS. 25 and 26 show the percent survival of mice with Daudi tumors (up to 90 days) after treatment with TRU-016 or rituximab. Treatment with TRU-016 increased median survival time in established Daudi tumors (FIG. 25). TRU-016 was more effective than rituximab in maintaining survival in mice with Daudi tumors (FIG. 26).

Administration of TRU-016 as a single agent in mice with established Ramos tumors demonstrated an inhibition of tumor growth and improved survival times equivalent to rituximab administered as a single agent, and was superior to HuIgG control-treated mice. Pooled data from 3 experiments demonstrated that TRU-016 and rituximab combination therapy resulted in a statistically significantly improvement in survival time compared to TRU-016 (p=0.028) or rituximab (p=0.045) monotherapies. Complete tumor regressions were also enhanced for the TRU-016 and rituximab combination groups. Forty-two percent of the TRU-016+rituximab 200 μg combination group were able to achieve long-term complete regression of their tumors compared to a 20% tumor regression rate in mice treated with either TRU-016 or rituximab alone (see Table 3 and FIG. 24).

TABLE 3
Survival after Treatment in Established Ramos Tumors

| | Percentage of Tumor- | Median Survival Time |
|---|---|---|
| | Free Mice at Day 90 | (Days) |
| TRU-016 + rituximab | 42 | 31 |
| (200 μg) | | |
| TRU-016 + rituximab | 25 | 24 |
| (100 μg) | | |
| TRU-016 (200 μg) | 20 | 16 |
| Rituximab (200 μg) | 20 | 17 |
| HuIgG | 0 | 10 |

Reduction in tumor growth and improved survival time were found after TRU-016 treatment in the Daudi tumor xenograft model (see Table 4 and FIGS. 25 and 26). TRU-016 administration significantly enhanced survival time compared to the control group. An increase in percentage of tumor-free mice was also observed with SMIP-016 treatment in this model compared to both control and rituximab groups.

TABLE 4
Survival after Treatment in Established Daudi Tumors

| | Percentage of Tumor | Median Survival Time |
|---|---|---|
| | Free Mice at Day 90 | (Days) |
| TRU-016 (100 μg) | 25 | 24 |
| Rituximab (100 μg) | 0 | 17 |
| HuIgG | 0 | 15 |

Treatment with a CD37-directed SMIP (TRU-016) is as effective as rituximab monotherapy in reducing tumor volume and increasing survival time in the Ramos tumor xenograft model. TRU-016+rituximab combination therapy demonstrated enhanced benefit in reducing tumor volume and significantly improving survival time compared to either rituximab or TRU-016 monotherapy in the Ramos tumor xenograft model. In the Daudi xenograft model, TRU-016-treated mice demonstrated a statistically significant increase in median survival time compared to HuIgG controls. Treatment with rituximab did not extend survival times compared to control mice. These data highlight the efficacy of a CD37-directed therapy in these NHL xenograft models.

### Example 15

### TRU-016 Potentiates Fludarabine-Induced Cell Death in CLL Cells In Vitro

Fludarabine is a chemotherapy drug used in the treatment of hematological malignancies. Fludarabine is a purine analog that inhibits DNA synthesis by interfering with ribonucleotide reductase and DNA polymerase. Fludarabine is active against both dividing and resting cells. Fludarabine is highly effective in the treatment of chronic lymphocytic leukemia (CLL), producing higher response rates than alkylating agents such as chlorambucil alone (Rai et al., N. Engl. J. Med. 343:1750-1757, 2000). Fludarabine is used in various combinations with cyclophosphamide, mitoxantrone, dexamethasone and rituximab in the treatment of indolent lymphoma and non-Hodgkins lymphoma. However, resistance to fludarabine has also been observed in treatment. Fludarabine induces caspase-dependent apoptosis in CLL cells, and apoptosis mediated by TRU-016 appears to be independent of caspase activation. The present study examined the effect of TRU-016 with fludarabine on CLL cells.

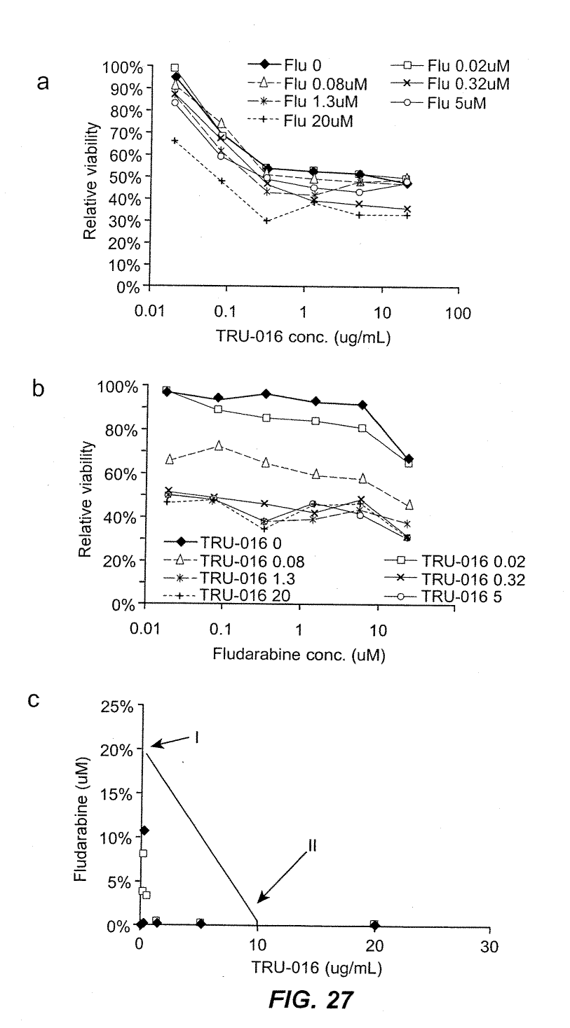

Cells were treated with TRU-016 at dosages ranging from 0.1-100 μg/mL and with fludarabine at dosages ranging from 0-20 μM (see FIG. 27). TRU-016 was provided by Trubion Pharmaceuticals (Seattle, Wash.). Fludarabine (F-araA) was purchased from SIGMA (St. Louis, Mo.). RPMI 1640 media was purchased from Invitrogen (Carlsbad, Calif.). Fluorescein isothiocyanate (FITC)-labeled annexin V, and propidium iodide (PI) were purchased from BD Pharmingen, San Diego, Calif. [3-(4,5-dimethylthiazol-2-yl)-2,5-diphenyltetrazolium bromide (MTT) was purchased from Sigma (St. Louis, Mo.). B-CLL cells were isolated immediately following donation using ficoll density gradient centrifugation (Ficoll-Paque Plus, Amersham Biosciences, Piscataway, N.J.). Isolated mononuclear cells were incubated in RPMI 1640 media supplemented with 10% heat-inactivated fetal bovine serum (FBS, Hyclone Laboratories, Logan, Utah), 2 mM L-glutamine (Invitrogen, Carlsbad, Calif.), and penicillin (100 U/mL)/streptomycin (100 μg/ml; Sigma-Aldrich, St. Louis) at 37° C. in an atmosphere of 5% CO₂. Freshly isolated B-CLL cells were used for all the experiments described herein except for the surface staining. For those samples with less than 90% B cells, negative selection was applied to deplete non-B cells using B cell Isolation Kit II (Miltenyi Biotec, Auburn, Calif.) or by “Rosette-Sep” kit from Stem Cell Technologies (Vancouver, British Columbia, Canada) according to the manufacture suggested protocol. Raji (Human Burkitt's lymphoma cell line) cell line was purchased from ATCC and maintained in RPMI 1640 media containing 10% FBS, supplemented with penicillin, streptomycin and glutamine. Cells were split 1:3 when the cell density reached 1×10⁶/mL. Media was changed the night before each study to assure fresh cells being used.

Cells were treated in vitro as described herein. 1:4 serial dilution of fludarabine (44, 11, 2.8, 0.7, 0.17 and 0.04 μM) was prepared in a 6-well plate by transferring 2 mL of drug-containing media to the next well containing 6 mL blank media. In a separate 6-well plate, 1:4 serial dilution of TRU-016 (44, 11, 2.8, 0.7, 0.17, and 0.04 μg/ml) in media was prepared using the same dilution method. From each of the plates, 0.45 mL media was transferred to a designed well in a 48-well plate to make a mixed drug solution in media (0.9 mL total in each well). Suspended CLL cells in media at a density of 1×10⁷ cells/mL (0.1 mL) were then added to the 0.9 mL media in each well to make a final density of 1×10⁶ cells/mL. For Raji cells, the final cell density was 5×10⁴ cells/mL. Thus, the cell suspension used was 5×10⁵ cells/mL. For the MTT assays, drug serial dilutions were prepared in 96-well plates, and transferred to other 96-well plates for incubation with cells. The total volume for incubation is 200 μL (90 μL of fludarabine solution, 90 μL of TRU-016 solution, and 20 μL cell suspension). Cell viability was assessed using MTT assays at 48 hr, and apoptosis was measured using Annexin V/PI at 24 hr.

MTT assays were performed to measure cell viability as described herein. Briefly, 10⁶ CLL cells were seeded to 96-well plates. Cells were incubated for 48 hours. 50 μl of MTT working solution (2 mg/ml, prepared from 5 mg/mL MTT reagent mixed with RPMI 16402:3 v/v) was added to each well, and the cells were incubated for 8 hours. Plates were centrifuged and supernatant was removed and dissolved in 100 μl lysis solution. Samples were measured with a plate reader at O.D.540. Cell viability was expressed as the percentage of viability compared with media control.

The apoptosis of CLL cells after incubation with antibodies was measured using annexin V-FITC/propidium iodide (PI) staining with FACS analysis. 5×10⁵ cells in 200 μl 1× binding buffer (BD Pharmingen) were stained with 5 μL annexin V (BD Pharmingen) and 5 μL PI (BD Pharmingen), and kept in the dark at room temperature for 15 minutes before suspension with 300 μl 1× buffer and analyzed by flow cytometry. Cells without staining, cells stained only with Annexin V, and cells stained only with PI were prepared. For all flow cytometry experiments, FACS analysis was performed using a Beckman-Coulter EPICS XL cytometer (Beckman-Coulter, Miami, Fla.). Fluorophores were excited at 488 nm. FITC-fluorescence was measured with FL1, while PI and PE fluorescence was measured with FL3. System II software package (Beckman-Coulter) was applied to analyze the data. The counted cell number was set at 10,000 for each sample.

A synergistic effect was determined by use of the isobologram method. To identify synergy, the effect of a drug combination was compared to the effect of each drug alone. This is based on the equation: Ca/Ca,b+Cb/Cb,a=Cl, where Ca and Cb are the concentration of drug A and drug B alone, respectively, to produce a desired effect (e.g. 50% cell death). Ca,b and Cb,a are the concentrations of drug A and drug B in a combination, respectively, to produce the same effect. Cl is the combination index. The concentrations of fludarabine and TRU-016, which elicit 50% death (IC50) were determined and are shown in FIG. 27C [IC50 of Fludarabine (I) and IC50 of TRU-016 (II)]. The straight line between these two points on the axes is the line of additive effect. Subsequently, different combinations of fludarabine and TRU-016 that achieve 50% cell death were also determined from the viability study and plotted to the same graph. When points fall below the additivity line, synergy is indicated. When points rise above the line, antagonism is indicated. When points are on the line, additivity is indicated.

FIG. 27 shows that TRU-016 effectively reduced relative cell viability in cells treated with fludarabine, thereby potentiating the cytotoxic effect of fludarabine alone. Thus, this study provides evidence that TRU-016 can be co-administered with fludarabine, resulting in increased effectiveness (i.e., synergistic reduction of CLL cells) in the treatment of hematological malignancies.

### Example 16

### TRU-016 Induces Direct Cytotoxicity in Rituximab-Resistant Cells

As disclosed herein, rituximab is a monoclonal antibody used in the treatment of NHL, FCC, MCL, DLCL, SLL, and CLL. The present study was undertaken to determine the efficacy of TRU-016 in inducing direct cytotoxicty in cells resistant to rituximab.

Rituximab-resistant cells (1×10⁶ cells) (Raji 4RH and RL 4RH, supplied by Dr. Myron S. Czuczman, Roswell Park Cancer Institute, Buffalo, N.Y.) were treated with herceptin (10 μg/mL), rituximab (10 μg/mL), or TRU-016 (5 μg/mL) in the presence of a five-fold excess of goat anti-human IgG for 24 hours. Direct cytoxicity was measured by annexin/PI staining and cell viability (percent) was calculated relative to control cells (cells treated with herceptin).

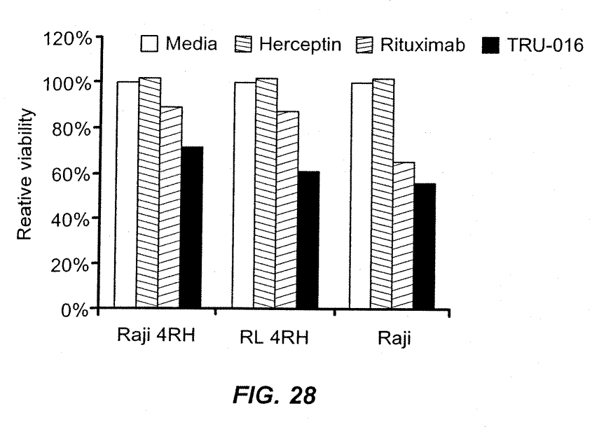

TRU-016 induced greater cell toxicity than rituximab in rituximab-resistant cell lines (see FIG. 28). Thus, TRU-016 is an effective agent for inducing cytoxicity in rituximab-resistant cells, making it useful as a therapeutic in diseases characterized by or involving rituximab-resistant cells, such as some B cells.

### Example 17

### TRU-016 Induces Tyrosine Phosphorylation in CD19+ Primary CLL B Cells

To determine how TRU-016 induces signal transduction in B cells, experiments were performed to examine the effect of TRU-016 on tyrosine phosphorylation.

Freshly isolated CD19+ cells (˜50−100×10⁶) from CLL patients were suspended at a concentration of 5×10⁶/ml PBS. Cells were then incubated for 10 minutes at 37° C., 5% CO₂, with control, trastuzumab (herceptin), or TRU-016 at a final concentration of 5 ug/ml. Cells were spun down, supernatant was removed, and cells were resuspended in fresh PBS of initial volume. Goat anti-human Fc fragment specific crosslinker (25 ug/ml) was added and cells were incubated for an additional 5 minutes. Cells were again spun down, supernatant was removed, and cells were lysed in 1 ml of RIPA lysis buffer with protease and phosphatase inhibitors (10 mM Tris, ph 7.4, 150 mM NaCl, 1% Triton X-100, 1% deoxycholic acid, 0.1% SDS and 5 mM EDTA all final concentrations. Sigma protease inhibitor cocktail cat# P-8340; Sigma phosphatase inhibitor cocktail: serine/threonine phosphatase inhibitor cocktail cat# P-2850; and tyrosine phosphatase inhibitor cat# P-5726; PMSF (100 mM) were all used. The inhibitors were added to the lysis buffer immediately prior to use at a 1:100 dilution. Protein concentration in the lysates was quantified by the bicin choninic acid (BCA) method (Pierce, Rockford, Ill.). The control and treated protein samples (50 ug total protein) were separated by two-dimensional gel electrophoresis (pH Range 3-10) (1st Dimension) and 10% SDS-PAGE (2nd Dimension). The protein was transferred to 0.2 Nm nitrocellulose membranes (Schleicher & Schuell, Keene, N.H.) and subjected to immunoblot analysis using anti-phosphotyrosine antibody clone 4G10 (Upstate Biotechnology), using standard protocol. Horseradish peroxidase (HRP)-conjugated goat anti-rabbit IgG was used as a secondary antibody. Detection of the phosphoprotein was made with chemiluminescent substrate (SuperSignal, Pierce Inc. Rockford, Ill.).

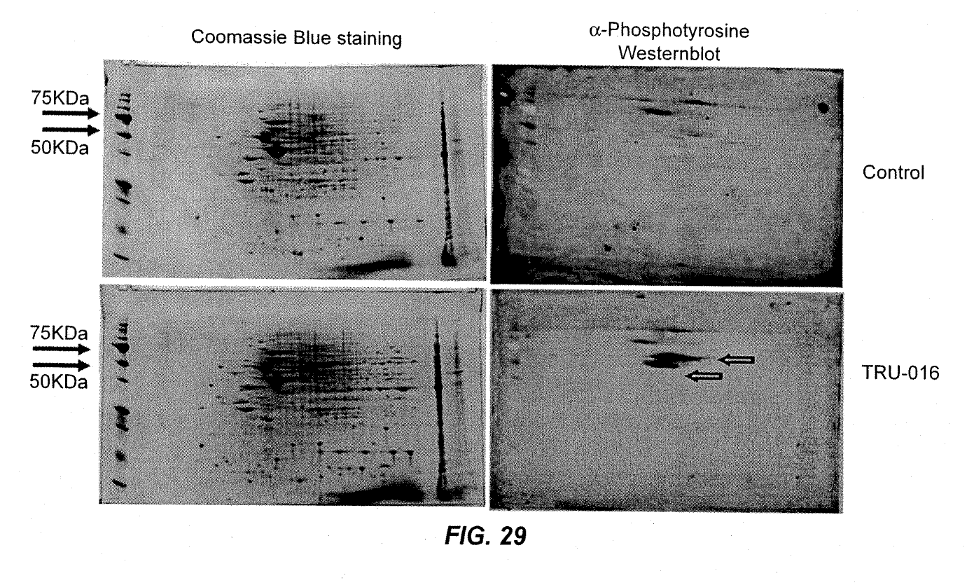

TRU-016 induced tyrosine phosphorylation in CD19+ primary CLL B cells, as shown by two-dimensional gel analysis (see FIG. 29). Thus, these experiments show that one way that TRU-016 acts is via a tyrosine phosphorylation pathway.

### Example 18

### Humanized TRU-016 Molecules

As set out in Example 1, CD37-specific SMIPs (such as TRU-016) are described in co-owned U.S. application Ser. No. 10/627,556 and U.S. Patent Application Publication Nos. 2003/133939, 2003/0118592 and 2005/0136049. Those descriptions are incorporated by reference herein. An exemplary CD37-specific SMIP, TRU-016 polypeptide (SEQ ID NO: 2), was produced and described therein. The present example provides humanized TRU-016 SMIPs.

Humanized antibodies are known in the art and are discussed in United States Patent Application Publication No. 2006/0153837. The present application uses the techniques involved in antibody humanization (discussed below) to humanize SMIPs, and particularly to humanize TRU-016.

“Humanization” is expected to result in an antibody that is less immunogenic, with complete retention of the antigen-binding properties of the original molecule. In order to retain all of the antigen-binding properties of the original antibody, the structure of its antigen binding site should be reproduced in the “humanized” version. This can be achieved by grafting only the nonhuman CDRs onto human variable framework domains and constant regions, with or without retention of critical framework residues (Jones et al, Nature 321:522 (1986); Verhoeyen et al, Science 239:1539 (1988)) or by recombining the entire nonhuman variable domains (to preserve ligand-binding properties), but “cloaking” them with a human-like surface through judicious replacement of exposed residues (to reduce antigenicity) (Padlan, Molec. Immunol. 28:489 (1991)).

Essentially, humanization by CDR grafting involves recombining only the CDRs of a non-human antibody onto a human variable region framework and a human constant region. Theoretically, this should substantially reduce or eliminate immunogenicity (except if allotypic or idiotypic differences exist). However, it has been reported that some framework residues of the original antibody also may need to be preserved (Reichmann et al, Nature, 332:323 (1988); Queen et al, Proc. Natl. Acad. Sci. USA, 86:10,029 (1989)).

The framework residues that need to be preserved are amenable to identification through computer modeling. Alternatively, critical framework residues may potentially be identified by comparing known antigen-binding site structures (Padlan, Molec. Immun., 31(3):169-217 (1994)), incorporated herein by reference.

The residues that potentially affect antigen binding fall into several groups. The first group comprises residues that are contiguous with the antigen site surface, which could therefore make direct contact with antigens. These residues include the amino-terminal residues and those adjacent to the CDRs. The second group includes residues that could alter the structure or relative alignment of the CDRs, either by contacting the CDRs or another peptide chain in the antibody. The third group comprises amino acids with buried side chains that could influence the structural integrity of the variable domains. The residues in these groups are usually found in the same positions (Padlan, 1994, supra) although their positions as identified may differ depending on the numbering system (see Kabat et al, “Sequences of proteins of immunological interest, 5th ed., Pub. No. 91-3242, U.S. Dept. Health & Human Services, NIH, Bethesda, Md., 1991).

Although the present invention is directed to the humanization of SMIPs and not antibodies, knowledge about humanized antibodies in the art is applicable to the SMIPs according to the invention. Some examples of humanized TRU-016 molecules are set out in Table 5 below.

To make humanized TRU-016 constructs of the invention, the mouse framework regions of TRU-016 were aligned to human VH1 and VH5 framework residues for the heavy chain and VK1 and VK3 for the light chain. Best matches were analyzed for framework compatibility with the CDRs of the mouse variable regions. Although there were several equally compatible combinations to chose from, we had previous success using the VK3 (X01668), VH5-51(Z12373) combination, so the humanized anti-CD37 SMIPs were designed using these human frameworks joined by a 15aa Gly₄Ser ((g4s)3) scFv linker. The VK3 construct was constructed with JK1 as a preferred FR4 match and the VH5 was constructed with JH2 coding for FR4, as with previously-described constructs. SMIPs were constructed de novo using overlapping oligonucleotide PCR. Full-length products were cloned into the SMIP expression vector in frame with the human IgG1 hinge, CH2, and CH3. These clones were sequence verified, transfected into COS-7 cells and 3-day conditioned media tested for binding to the B-cell lymphoma line, Ramos. In order to increase humanization, changes were incorporated into CDR1 of the light chain at positions L25, L27 and L28 and were well tolerated, showing equal binding activity with the original humanized molecule 019001. Further DNA constructs were made in a similar fashion to alter the CDR3 of the VH region by incorporating germline amino acids, H100-H102, encoded by various human JH regions. Constructs were examined for expression level and degree of binding to CD37 on Ramos cells.

An improved binding affinity was found after simultaneously changing the V region orientation to VH-VL and lengthening the linker between the V regions to 25 amino acids, as was done in SEQ ID NO: 222 (see Table 5). These changes resulted in binding affinity like that of SMIP-016. Thus, these changes resulted in a binding affinity that was about 2-4 fold greater than versions of the molecule with the VL-15aa linker-VH.

TABLE 5
Humanized TRU-016 Constructs

| Construct | | | | DNA SEQ | AA SEQ |
|---|---|---|---|---|---|
| No. | Description | Linker | Hinge | ID NO: | ID NO: |
| 019001 | Vk3: VH5-51 | 15aa | SSC-P | 5 | 6 |
| | | gly4ser | | | |
| 019002 | Vk3: VH5-51 | 15aa | SSC-P | 7 | 8 |
| | Linker (TG-SS) | gly4ser | | | |
| 019003 | Vk3: VH5-51 | 15aa | SSC-P | 9 | 10 |
| | VH V11S | gly4ser | | | |
| 019004 | Vk3: VH5-51 | 15aa | SSC-P | 11 | 12 |
| | VK3, cdr1 (E | gly4ser | | | |
| | →Q) | | | | |
| 019005 | Vk3: VH5-51 | 15aa | SSC-P | 13 | 14 |
| | VK3, cdr1 (N → | gly4ser | | | |
| | S) | | | | |
| 019006 | Vk3: VH5-51 | 15aa | SSC-P | 15 | 16 |
| | VK3, cdr1 (T → | gly4ser | | | |
| | A) | | | | |
| 019010 | mVk: VH5-5a | 15aa | SSC-P | 17 | 18 |
| | | gly4ser | | | |
| 019011 | Vk3: mVH | 15aa | SSC-P | 19 | 20 |
| | (linker G-S | gly4ser | | | |
| | mutation) | | | | |
| 019017 | Vk3: VH5 VH3 | 15aa | SSC-P | 21 | 22 |
| | FW1 | gly4ser | | | |
| 019018 | mVH: Vk3 | 15aa | SSC-P | 23 | 24 |
| | | gly4ser | | | |
| 019019 | Vk3: mVH | 15aa | SSC-P | 25 | 26 |
| | (019011 with | gly4ser | | | |
| | 2H7 Leader) | | | | |
| 019021 | mVH: Vk3 | 15aa | SSC-P | 27 | 28 |
| | | gly4ser | | | |
| 019023 | Vk3: mVH | 15aa | SSC-P | 29 | 30 |
| | (fixed 019011 | gly4ser | | | |
| | GS4 mutation) | | | | |
| 019024 | Vk3: mVH | 15aa | SSC-P | 31 | 32 |
| | (fixed 019011 | gly4ser | | | |
| | GS4 mutation) | | | | |
| 019025 | Vk3: VH5 VH3 | 15aa | SSC-P | 33 | 34 |
| | FW1 | gly4ser | | | |
| 019026 | Vk3: VH5 VH3 | 15aa | SSC-P | 35 | 36 |
| | FW1 | gly4ser | | | |
| 019032 | Vk3: VH5 VH3- | 15aa | SSC-P | 37 | 38 |
| | 13 FW1 | gly4ser | | | |
| 019033 | Vk3: VH5 VH3- | 15aa | SSC-P | 39 | 40 |
| | 13 FW1 | gly4ser | | | |
| 019034 | Vk3: VH5 VH3- | 15aa | SSC-P | 41 | 42 |
| | 13 L11S FW1 | gly4ser | | | |
| 019035 | Vk3: VH5 VH3- | 15aa | SSC-P | 43 | 44 |
| | 13 L11S FW1 | gly4ser | | | |
| 019037 | Vk3(CDR-L1 | 15aa | SSC-P | 45 | 46 |
| | changes): VH5 | gly4ser | | | |
| 019041 | 019006 - | 15aa | SSC-P | 47 | 48 |
| | CDR-H3 JH4 | gly4ser | | | |
| 019043 | 019006 - | 15aa | SSC-P | 49 | 50 |
| | CDR-H3 JH6 | gly4ser | | | |
| 019044 | 019006 - | 15aa | SSC-P | 51 | 52 |
| | CDR-H3 JH5a | gly4ser | | | |
| 019045 | 019006 - | 15aa | SSC-P | 53 | 54 |
| | CDR-H3 JH5b | gly4ser | | | |
| 019046 | 019006 - | 15aa | SSC-P | 55 | 56 |
| | CDR-H3 JH1 | gly4ser | | | |
| 019047 | 019006 - | 15aa | SSC-P | 57 | 58 |
| | CDR-H3 JH3a | gly4ser | | | |
| 019048 | 019006 - CDR- | 15aa | SSC-P | 59 | 60 |
| | H3 JH3b | gly4ser | | | |
| 019049 | 019006 - CDR- | 15aa | SSC-P | 79 | 80 |
| | H3 JH2 | gly4ser | | | |
| 019050 | 019006 - CDR- | 15aa | SSC-P | 81 | 82 |
| | H2 changes | gly4ser | | | |
| 019051 | 019044 | 20aa | CPPCP | 83 | 84 |
| | | gly4ser | | | |
| 019008 | | | | 85 | 86 |
| 019009 | | | | 87 | 88 |
| | | 25aa | | 221 | 222 |
| | | gly4ser | | | |

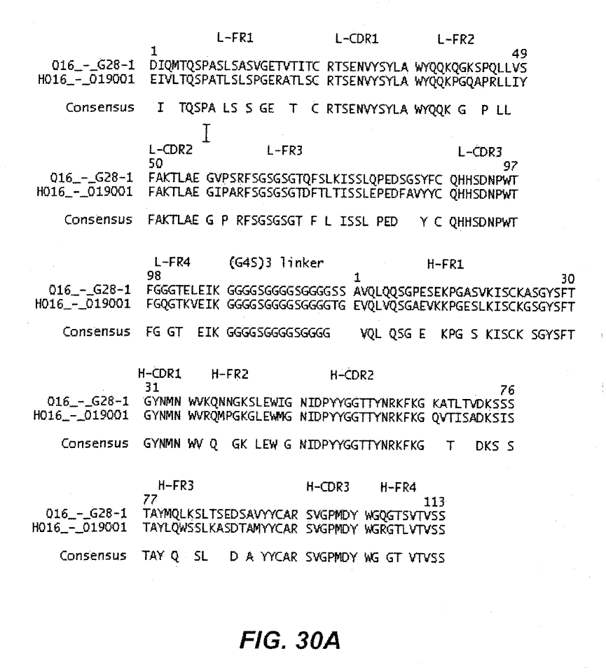

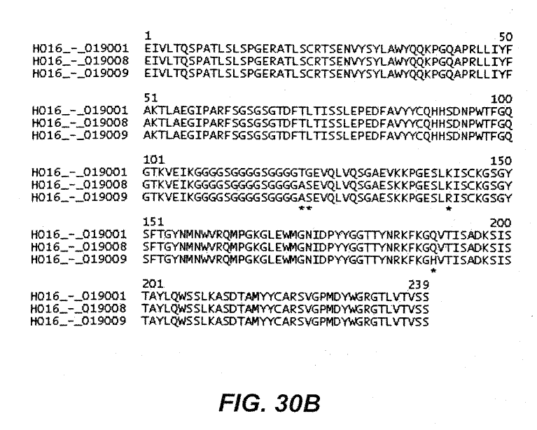

The amino acid consensus sequence of humanized TRU-016 construct no. 019001 (SEQ ID NO: 6; H016-019001) and non-humanized TRU-016 (SEQ ID NO: 2; 016-G28-1) is shown with Kabat numbering in FIG. 30A. FIG. 30 B shows the amino acid sequence alignments of humanized TRU-016 construct nos. 019001 (SEQ ID NO: 6), 019008 (SEQ ID NO: 86), and 019009 (SEQ ID NO: 88).

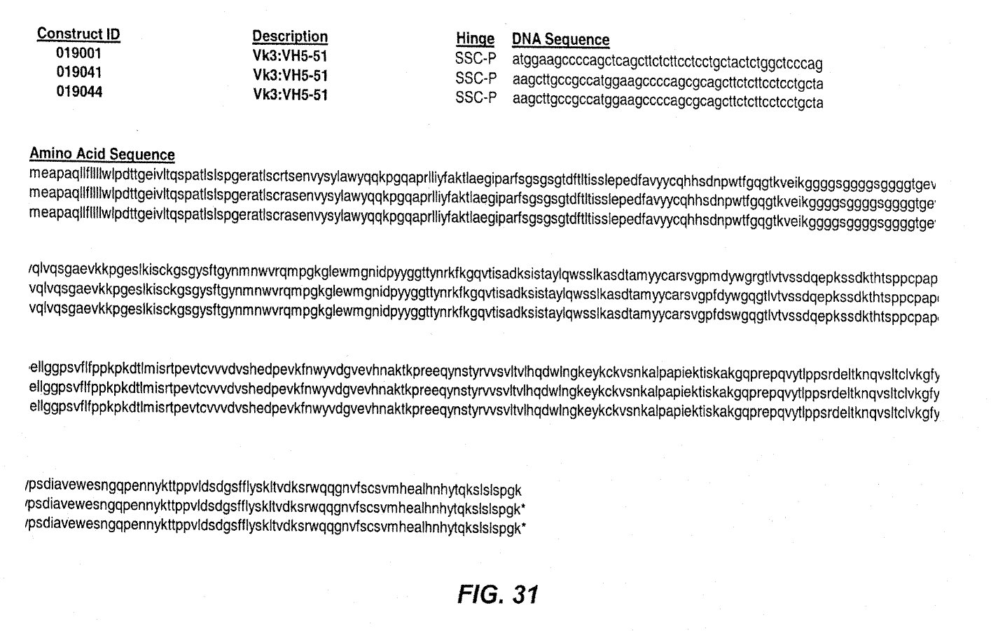

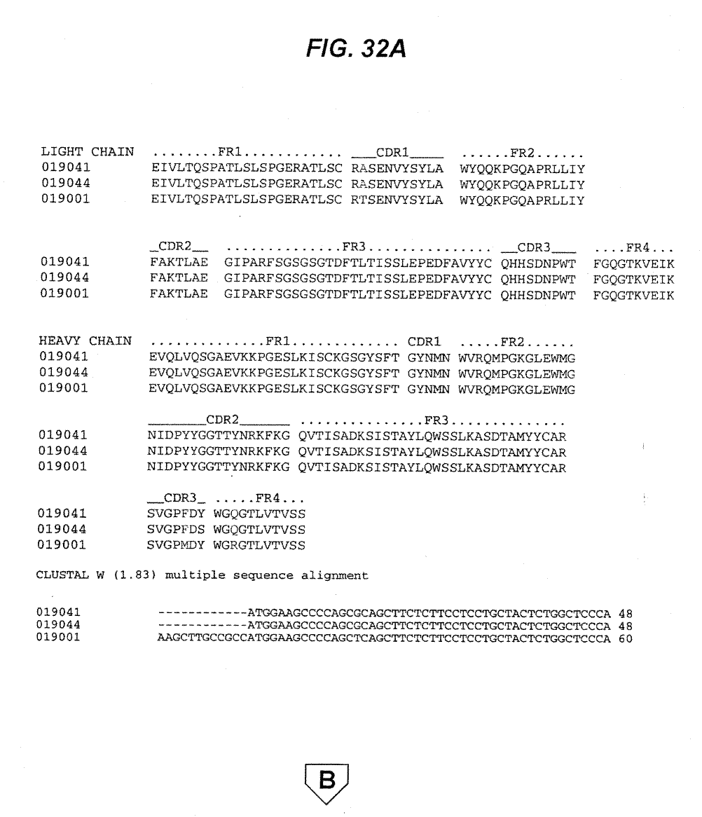

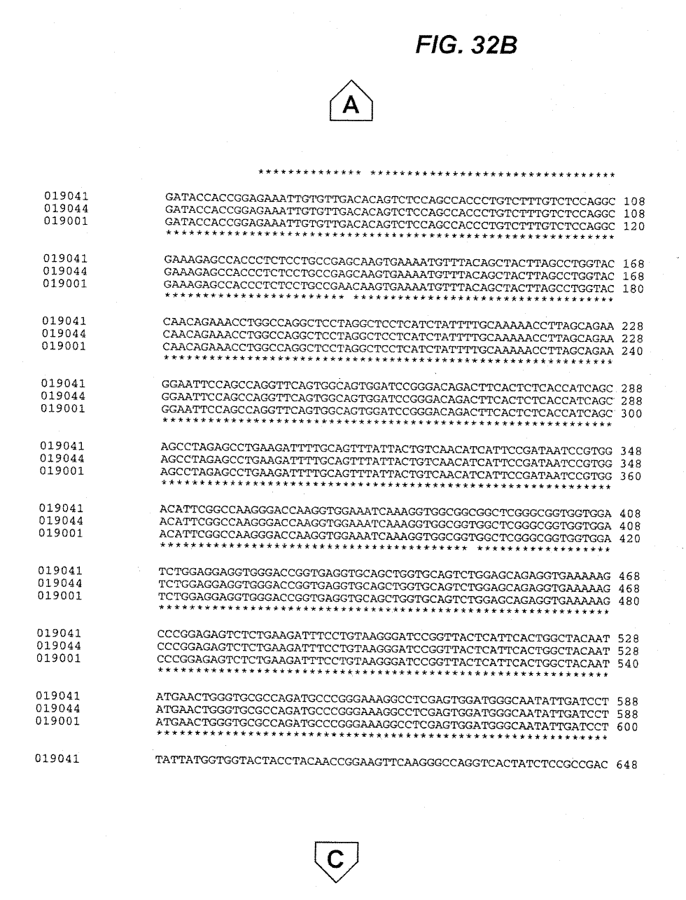

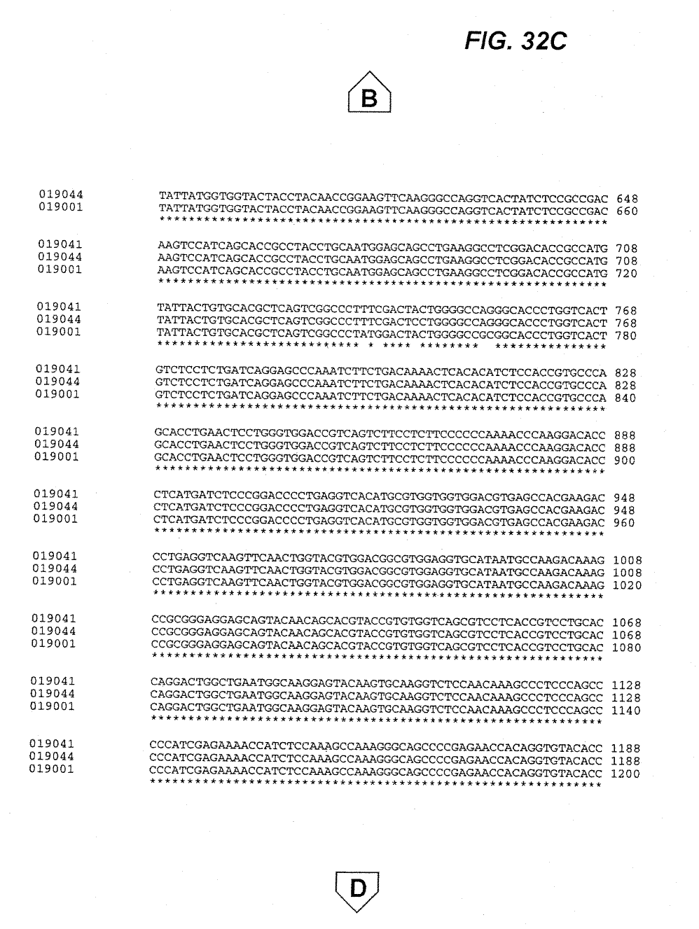

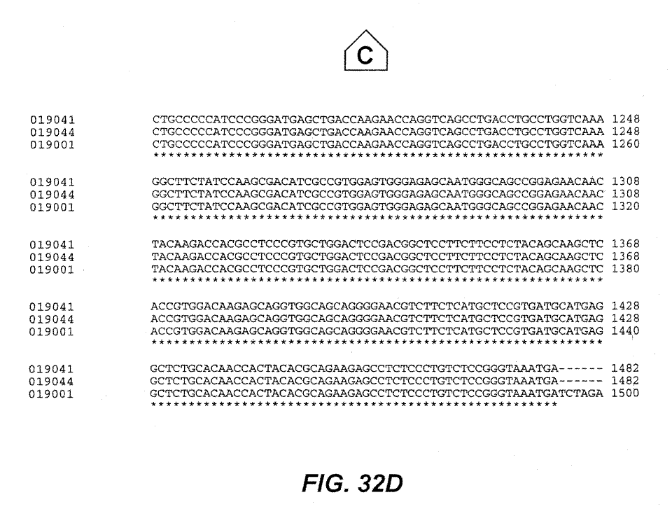

DNA and amino acid sequence alignments of three humanized constructs of TRU-016 (019001, 019041, and 019044), demonstrating high CD37-specific binding to Ramos B cells are shown in FIG. 31. FASTA formatted DNA and amino acid sequence alignments of the same three humanized constructs of TRU-016 (019001, 019041, and 019044) are shown in FIG. 32.

Additional hinge regions (Table 6) and framework regions (Table 7) that may be used in the humanized TRU-016 molecules of the invention are provided below.

TABLE 6
Hinge Regions for Humanized TRU-016 SMIPs

| | | |
|---|---|---|
| | | SEQ |
| Hinge | | ID |
| description | DNA or Amino Acid Sequence | NO: |

| | | |
|---|---|---|
| ccc(p)- | gagcccaaatcttgtgacaaaactcacacatgtc | 89 |
| hlgG1 | caccgtgccca | |
| (DNA) | | |
| ccc(p)- | EPKSCDKTHTCPPCP | 90 |
| hlgG1 (AA | | |
| scc(p)- | gagcccaaatcttctgacaaaactcacacatgtc | 91 |
| hlgG1 | caccgtgccca | |
| (DNA) | | |
| scc(p)- | EPKSSDKTHTCPPCP | 92 |
| hlgG1 (AA) | | |
| scc(s)- | gagcccaaatcttctgacaaaactcacacatgtc | 93 |
| hlgG1 | caccgtgctca | |
| (DNA) | | |
| scc(s)- | EPKSSDKTHTCPPCS | 94 |
| hlgG1 (AA) | | |
| scs(s)- | gagcccaaatcttgtgacaaaactcacacatgtc | 95 |
| hlgG1 | caccgagctca | |
| (DNA) | | |
| scs(s)- | EPKSSDKTHTCPPSS | 96 |
| hlgG1 (AA) | | |
| sss(p)- | gagcccaaatcttctgacaaaactcacacatctc | 97 |
| hlgG1 | caccgagccca | |
| (DNA) | | |
| sss(p)- | EPKSSDKTHTSPPSP | 98 |
| hlgG1 (AA) | | |
| sss(s)- | gagcccaaatcttctgacaaaactcacacatctc | 99 |
| hlgG1 | caccgagctca | |
| (DNA) | | |
| sss(s)- | EPKSSDKTHTSPPSS | 100 |
| hlgG1 (AA) | | |
| csc(p)- | gagcccaaatcttgtgacaaaactcacacatctc | 101 |
| hlgG1 | caccgtgccca | |
| (DNA) | | |
| csc(p)- | EPKSCDKTHTSPPCP | 102 |
| hlgG1 (AA) | | |
| csc(s)- | gagcccaaatcttgtgacaaaactcacacatctc | 103 |
| hlgG1 | caccgtgctca | |
| (DNA) | | |
| csc(s)- | EPKSCDKTHTSPPCS | 104 |
| hlgG1 (AA) | | |
| ssc(p)- | gagcccaaatcttctgacaaaactcacacatctc | 105 |
| hlgG1 | caccgtgccca | |
| (DNA) | | |
| ssc(p)- | EPKSSDKTHTSPPCP | 106 |
| hlgG1 (AA) | | |
| scs(s)- | gagcccaaatcttctgacaaaactcacacatctc | 107 |
| hlgG1 | caccgtgctca | |
| (DNA) | | |
| scs(s)- | EPKSSDKTHTSPPCS | 108 |
| hlgG1 (AA) | | |
| css(p)- | gagcccaaatcttgtgacaaaactcacacatctc | 109 |
| hlgG1 | caccgagccca | |
| (DNA) | | |
| css(p)- | EPKSCDKTHTSPPSP | 110 |
| hlgG1 (AA) | | |
| css(s)- | gagcccaaatcttgtgacaaaactcacacatctc | 111 |
| hlgG1 | caccgagctca | |
| (DNA) | | |
| css(s)- | EPKSCDKTHTSPPSS | 112 |
| hlgG1 (AA) | | |
| scs(s)- | gagcccaaatcttgtgacaaaactcacacatgtc | 113 |
| hlgG1 | caccgagctca | |
| (DNA) | | |
| scs(s)- | EPKSSDKTHTCPPSS | 114 |
| hlgG1 (AA) | | |
| hlgA1 | VPSTPPTPSPSTPPTPSPS | 115 |
| hlgA2 | VPPPPP | 116 |
| hlgG3 | gagctcaaaactcctctcggggatacgacccata | 117 |
| (DNA) | cgtgtccccgctgtcctgaaccgaagtcctgcga | |
| | tacgcctccgccatgtccacggtgcccagagccc | |
| | aaatcatgcgatacgcccccaccgtgtccccgct | |
| | gtcctgaaccaaagtcatgcgataccccaccacc | |
| | atgtccaagatgccca | |
| hlgG3 (AA) | ELKTPLGDTTHTCPRCPEPKSCDTPPPCPRCP | 118 |
| | EPKSCDTPPPCPRCPEPKSCDTPPPCPRCP | |
| lgG315hscc | gagcccaaatcttctgacacacctcccccatgcc | 119 |
| (DNA) | cacggtgcccc | |
| lgG315hscc | EPKSSDTPPPCPRCP | 120 |
| (AA) | | |
| lgG315hcss | gagcccaaatcttgtgacacacctcccccatccc | 121 |
| (DNA) | cacggtcccca | |
| lgG315hcss | EPKSCDTPPPSPRSP | 122 |
| (AA) | | |
| lgG315hsss | gagcccaaatcttctgacacacctcccccatccc | 123 |
| (DNA) | cacggtcccca | |
| lgG315hsss | EPKSSDTPPPSPRSP | 124 |
| (AA) | | |
| lgG3hl5csc | gagcccaaatcttgtgacacacctcccccatccc | 125 |
| (DNA) | cacggtgccca | |
| lgG3hl5csc | EPKSCDTPPPSPRCP | 126 |
| (AA) | | |
| hlgD | ESPKAQASSVPTAQPQAEGSLAKATTAPATTR | 127 |
| | NTGRGGEEKKKEKEKEEQEERETKTP | |

TABLE 7
Framework Regions for Humanized TRU-016 SMIPs

| | |
|---|---|
| | SEQ |
| | ID |
| V-region | NO: |

Human VH Framework Regions for
anti-CD37 Humanization
FR1

| | | |
|---|---|---|
| VH1 | QVQLVQSGAEVKKPGASVKVSCKASGYTFT | 140 |
| VH1 | QVQLVQSGAEVKKPGSSVKVSCKASGGTFS | 141 |
| VH1 | QVQLVQSGAEVKKPGSSVKVSCKASGGTFS | 142 |
| VH1 | EVQLVQSGAEVKKPGATVKISCKVSGYTFT | 143 |
| VH5 | EVQLVQSGAEVKKPGESLKISCKGSGYSFT | 144 |
| VH5 | EVQLVQSGAEVKKPGESLRISCKGSGYSFT | 145 |
| VH7 | QVQLVQSGSELKKPGASVKVSCKASGYTFT | 146 |

FR2

| | | |
|---|---|---|
| VH1 | WVRQAPGQGLEWMG | 147 |
| VH1 | WVRQAPGQGLEWMG | 148 |
| VH1 | WVRQAPGQGLEWMG | 149 |
| VH1 | WVQQAPGKGLEWMG | 150 |
| VH5 | WVRQMPGKGLEWMG | 151 |
| VH5 | WVRQMPGKGLEWMG | 152 |
| VH7 | WVRQAPGQGLEWMG | 153 |

FR3

| | | |
|---|---|---|
| VH1 | RVTMTTDTSTSTAYMELRSLRSDDTAVYYCAR | 154 |
| VH1 | RVTITADESTSTAYMELSSLRSEDTAVYYCAR | 155 |
| VH1 | RVTITADKSTSTAYMELSSLRSEDTAVYYCAR | 156 |
| VH1 | RVTITADTSTDTAYMELSSLRSEDTAVYYCAT | 157 |
| VH5 | QVTISADKSISTAYLQWSSLKASDTAMYYCAR | 158 |
| VH5 | HVTISADKSISTAYLQWSSLKASDTAMYYCAR | 159 |
| VH7 | RFVFSLDTSVSTAYLQISSLKAEDTAVYYCAR | 160 |

FR4

| | |
|---|---|
| WGQGTLVTVSS | 161 |
| WGRGTLVTVSS | 162 |
| WGQGTMVTVSS | 163 |
| WGQGTMVTVSS | 164 |
| WGQGTLVTVSS | 165 |
| WGQGTLVTVSS | 166 |
| WGQGTLVTVSS | 167 |
| WGQGTTVTVSS | 168 |
| WGKGTTVTVSS | 169 |

Human VK Framework Regions for
anti-CD37 Humanization
FR1

| | | |
|---|---|---|
| VK3 | EIVMTQSPATLSVSPGERATLSC | 170 |
| VK3 | EIVLTQSPATLSLSPGERATLSC | 171 |
| VK1 | DIQMTQSPSSLSASVGDRVTITC | 172 |
| VK1 | DIQMTQSPSSLSASVGDRVTITC | 173 |
| VK1 | DIQMTQSPSSLSASVGDRVTITC | 174 |
| VK1 | NIQMTQSPSAMSASVGDRVTITC | 175 |
| VK1 | DIQMTQSPSSLSASVGDRVTITC | 176 |
| VK1 | AIQLTQSPSSLSASVGDRVTITC | 177 |
| VK1 | DIQLTQSPSFLSASVGDRVTITC | 178 |
| VK1 | AIRMTQSPFSLSASVGDRVTITC | 179 |
| VK1 | AIQMTQSPSSLSASVGDRVTITC | 180 |
| VK1 | DIQMTQSPSTLSASVGDRVTITC | 181 |

FR2

| | | |
|---|---|---|
| VK3 | WYQQKPGQAPRLLIY | 182 |
| VK3 | WYQQKPGQAPRLLIY | 183 |
| VK1 | WYQQKPGKAPKLLIY | 184 |
| VK1 | WYQQKPGKVPKLLIY | 185 |
| VK1 | WYQQKPGKAPKRLIY | 186 |
| VK1 | WFQQKPGKVPKHLIY | 187 |
| VK1 | WFQQKPGKAPKSLIY | 188 |
| VK1 | WYQQKPGKAPKLLIY | 189 |
| VK1 | WYQQKPGKAPKLLIY | 190 |
| VK1 | WYQQKPAKAPKLFIY | 191 |
| VK1 | WYQQKPGKAPKLLIY | 192 |
| VK1 | WYQQKPGKAPKLLIY | 193 |

FR3

| | | |
|---|---|---|
| VK3 | GIPARFSGSGSGTEFTLTISSLQSEDFAVYYC | 194 |
| VK3 | GIPARFSGSGSGTDFTLTISSLEPEDFAVYYC | 195 |
| VK1 | GVPSRFSGSGSGTDFTLTISSLQPEDFATYYC | 196 |
| VK1 | GVPSRFSGSGSGTDFTLTISSLQPEDVATYYC | 197 |
| VK1 | GVPSRFSGSGSGTEFTLTISSLQPEDFATYYC | 198 |
| VK1 | GVPSRFSGSGSGTEFTLTISSLQPEDFATYYC | 199 |
| VK1 | GVPSRFSGSGSGTDFTLTISSLQPEDFATYYC | 200 |
| VK1 | GVPSRFSGSGSGTDFTLTISSLQPEDFATYYC | 201 |
| VK1 | GVPSRFSGSGSGTEFTLTISSLQPEDFATYYC | 202 |
| VK1 | GVPSRFSGSGSGTDYTLTISSLQPEDFATYYC | 203 |
| VK1 | GVPSRFSGSGSGTDFTLTISSLQPEDFATYYC | 204 |
| VK1 | GVPSRFSGSGSGTEFTLTISSLQPDDFATYYC | 205 |

FR4

| | |
|---|---|
| FGQGTKVEIK | 206 |
| FGQGTKLEIK | 207 |
| FGPGTKVDIK | 208 |
| FGGGTKVEIK | 209 |
| FGQGTRLEIK | 210 |

TABLE 8
DNA and Amino Acid Sequences for SEQ ID NOS: 79-88 and 221-222

| | | |
|---|---|---|
| Construct | SEQ ID | |
| # | NO: | DNA or Amino Acid Sequence |

| | | |
|---|---|---|
| 019049 | 79 | aagcttgccgccatggaagccccagcgcagcttctcttcctcctgctactctggctcccag |
| | | ataccaccggagaaattgtgttgacacagtctccagccaccctgtctttgtctccaggcga |
| | | aagagccaccctctcctgccgagcaagtgaaaatgtttacagctacttagcctggtacca |
| | | acagaaacctggccaggctcctaggctcctcatctattttgcaaaaaccttagcagaagg |
| | | aattccagccaggttcagtggcagtggatccgggacagacttcactctcaccatcagca |
| | | gcctagagcctgaagattttgcagtttattactgtcaacatcattccgataatccgtggacat |
| | | tcggccaagggaccaaggtggaaatcaaaggtggcggtggctcgggcggtggtggat |
| | | ctggaggaggtgggaccggtgaggtgcagctggtgcagtctggagcagaggtgaaaa |
| | | agcccggagagtctctgaagatttcctgtaagggatccggttactcattcactggctacaa |
| | | tatgaactgggtgcgccagatgcccgggaaaggcctcgagtggatgggcaatattgatc |
| | | cttattatggtggtactacctacaaccggaagttcaagggccaggtcactatctccgccga |
| | | caagtccatcagcaccgcctacctgcaatggagcagcctgaaggcctcggacaccgc |
| | | catgtattactgtgcacgctcagtcggccctttcgacctctggggcagaggcaccctggtc |
| | | actgtctcctctgatcaggagcccaaatcttctgacaaaactcacacatctccaccgtgcc |
| | | cagcacctgaactcctgggtggaccgtcagtcttcctcttccccccaaaacccaaggac |
| | | accctcatgatctcccggacccctgaggtcacatgcgtggtggtggacgtgagccacga |
| | | agaccctgaggtcaagttcaactggtacgtggacggcgtggaggtgcataatgccaag |
| | | acaaagccgcgggaggagcagtacaacagcacgtaccgtgtggtcagcgtcctcacc |
| | | gtcctgcaccaggactggctgaatggcaaggagtacaagtgcaaggtctccaacaaa |
| | | gccctcccagcccccatcgagaaaaccatctccaaagccaaagggcagccccgaga |
| | | accacaggtgtacaccctgcccccatcccgggatgagctgaccaagaaccaggtcag |
| | | cctgacctgcctggtcaaaggcttctatccaagcgacatcgccgtggagtgggagagca |
| | | atgggcagccggagaacaactacaagaccacgcctcccgtgctggactccgacggct |
| | | ccttcttcctctacagcaagctcaccgtggacaagagcaggtggcagcaggggaacgt |
| | | cttctcatgctccgtgatgcatgaggctctgcacaaccactacacgcagaagagcctctc |
| | | cctgtctccgggtaaatgatctaga |
| 019049 | 80 | MEAPAQLLFLLLLWLPDTTGEIVLTQSPATLSLSPGERATLSCR |
| | | ASENVYSYLAWYQQKPGQAPRLLIYFAKTLAEGIPARFSGSGS |
| | | GTDFTLTISSLEPEDFAVYYCQHHSDNPWTFGQGTKVEIKGGG |
| | | GSGGGGSGGGGTGEVQLVQSGAEVKKPGESLKISCKGSGYS |
| | | FTGYNMNWVRQMPGKGLEWMGNIDPYYGGTTYNRKFKGQ |
| | | VTISADKSISTAYLQWSSLKASDTAMYYCARSVGPFDLWGRGT |
| | | LVTVSSDQEPKSSDKTHTSPPCPAPELLGGPSVFLFPPKPKDT |
| | | LMISRTPEVTCVVVDVSHEDPEVKFNWYVDGVEVHNAKTKPR |
| | | EEQYNSTYRVVSVLTVLHQDWLNGKEYKCKVSNKALPAPIEKT |
| | | ISKAKGQPREPQVYTLPPSRDELTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPGK |
| 019050 | 81 | aagcttgccgccatggaagccccagctcagcttctcttcctcctgctactctggctcccag |
| | | ataccaccggagaaattgtgttgacacagtctccagccaccctgtctttgtctccaggcga |
| | | aagagccaccctctcctgccgagcaagtgaaaatgtttacagctacttagcctggtacca |
| | | acagaaacctggccaggctcctaggctcctcatctattttgcaaaaaccttagcagaagg |
| | | aattccagccaggttcagtggcagtggatccgggacagacttcactctcaccatcagca |
| | | gcctagagcctgaagattttgcagtttattactgtcaacatcattccgataatccgtggacat |
| | | tcggccaagggaccaaggtggaaatcaaaggtggcggtggctcgggcggtggtggat |
| | | ctggaggaggtggggctagcgaggtgcagctggtgcagtctggagcagaggtgaaaa |
| | | agcccggagagtctctgaagatttcctgtaagggatccggttactcattcactagctacaa |
| | | tatgaactgggtgcgccagatgcccgggaaaggcctggagtggatgggcaatattgat |
| | | ccttattatggtggtactaactacgcccagaagttccagggccaggtcactatctccgccg |
| | | acaagtccatcagcaccgcctacctgcaatggagcagcctgaaggcctcggacaccg |
| | | ccatgtattactgtgcacgctcagtcggccctatggactactggggccgcggcaccctgg |
| | | tcactgtctcctctgatcaggagcccaaatcttctgacaaaactcacacatctccaccgtg |
| | | cccagcacctgaactcctgggtggaccgtcagtcttcctcttccccccaaaacccaagg |
| | | acaccctcatgatctcccggacccctgaggtcacatgcgtggtggtggacgtgagccac |
| | | gaagaccctgaggtcaagttcaactggtacgtggacggcgtggaggtgcataatgcca |
| | | agacaaagccgcgggaggagcagtacaacagcacgtaccgtgtggtcagcgtcctc |
| | | accgtcctgcaccaggactggctgaatggcaaggagtacaagtgcaaggtctccaac |
| | | aaagccctcccagcccccatcgagaaaaccatctccaaagccaaagggcagccccg |
| | | agaaccacaggtgtacaccctgcccccatcccgggatgagctgaccaagaaccaggt |
| | | cagcctgacctgcctggtcaaaggcttctatccaagcgacatcgccgtggagtgggaga |
| | | gcaatgggcagccggagaacaactacaagaccacgcctcccgtgctggactccgac |
| | | ggctccttcttcctctacagcaagctcaccgtggacaagagcaggtggcagcagggga |
| | | acgtcttctcatgctccgtgatgcatgaggctctgcacaaccactacacgcagaagagc |
| | | ctctccctgtctccgggtaaatga |
| 019050 | 82 | MEAPAQLLFLLLLWLPDTTGEIVLTQSPATLSLSPGERATLSCR |
| | | ASENVYSYLAWYQQKPGQAPRLLIYFAKTLAEGIPARFSGSGS |
| | | GTDFTLTISSLEPEDFAVYYCQHHSDNPWTFGQGTKVEIKGGG |
| | | GSGGGGSGGGGASEVQLVQSGAEVKKPGESLKISCKGSGYS |
| | | FTSYNMNWVRQMPGKGLEWMGNIDPYYGGTNYAQKFQGQ |
| | | VTISADKSISTAYLQWSSLKASDTAMYYCARSVGPMDYWGRG |
| | | TLVTVSSDQEPKSSDKTHTSPPCPAPELLGGPSVFLFPPKPKD |
| | | TLMISRTPEVTCVVVDVSHEDPEVKFNWYVDGVEVHNAKTKP |
| | | REEQYNSTYRVVSVLTVLHQDWLNGKEYKCKVSNKALPAPIEK |
| | | TISKAKGQPREPQVYTLPPSRDELTKNQVSLTCLVKGFYPSDIA |
| | | VEWESNGQPENNYKTTPPVLDSDGSFFLYSKLTVDKSRWQQ |
| | | GNVFSCSVMHEALHNHYTQKSLSLSPGK |
| 019051 | 83 | aagcttgccgccatggaagccccagcgcagcttctcttcctcctgctactctggctcccag |
| | | ataccaccggagaaattgtgttgacacagtctccagccaccctgtctttgtctccaggcga |
| | | aagagccaccctctcctgccgagcaagtgagaatgtttacagctacttagcctggtacca |
| | | acagaaacctggccaggctcctaggctcctcatctattttgcaaaaaccttagcagaagg |
| | | gattccagccagattcagtggcagtggttccgggacagacttcactctcaccatcagcag |
| | | cctagagcctgaagattttgcagtttattactgtcaacatcattccgataatccgtggacatt |
| | | cggccaagggaccaaggtggaaatcaaaggtggcggtggctcgggcggtggtggat |
| | | ctggaggaggtgggagcggaggaggagctagcgaggtgcagctggtgcagtctgga |
| | | gcagaggtgaaaaagcccggagagtctctgaagatttcctgtaagggatccggttactc |
| | | attcactggctacaatatgaactgggtgcgccagatgcccgggaaaggcctcgaatgg |
| | | atgggcaatattgatccttattatggtggtactacctacaaccggaagttcaagggccagg |
| | | tcactatctccgccgacaagtccatcagcaccgcctacctgcaaggagcagcctgaag |
| | | gcctcggacaccgccatgtattactgtgcacgctcagtcggccctttcgactcctggggcc |
| | | agggcaccctggtcactgtctcgagttgtccaccgtgcccagcacctgaactcctgggtg |
| | | gaccgtcagtcttcctcttccccccaaaacccaaggacaccctcatgatctccggaccc |
| | | ctgaggtcacatgcgtggtggtggacgtgagccacgaagaccctgaggtcaagttcaa |
| | | ctggtacgtggacggcgtggaggtgcataatgccaagacaaagccgcgggaggagc |
| | | agtacaacagcacgtaccgtgtggtcagcgtcctcaccgtcctgcaccaggactggctg |
| | | aatggcaaggagtacaagtgcaaggtctccaacaaagccctcccagcccccatcgag |
| | | aaaaccatctccaaagccaaagggcagccccgagaaccacaggtgtacaccctgcc |
| | | cccatcccgggatgagctgaccaagaaccaggtcagcctgacctgcctggtcaaagg |
| | | cttctatccaagcgacatcgccgtggagtgggagagcaatgggcagccggagaacaa |
| | | ctacaagaccacgcctcccgtgctggactccgacggctccttcttcctctacagcaagctc |
| | | accgtggacaagagcaggtggcagcaggggaacgtcttctcatgctccgtgatgcatg |
| | | aggctctgcacaaccactacacgcagaagagcctctccctgtctccgggtaaatgactc |
| | | taga |
| 019051 | 84 | MEAPAQLLFLLLLWLPDTTGEIVLTQSPATLSLSPGERATLSCR |
| | | ASENVYSYLAWYQQKPGQAPRLLIYFAKTLAEGIPARFSGSGS |
| | | GTDFTLTISSLEPEDFAVYYCQHHSDNPWTFGQGTKVEIKGGG |
| | | GSGGGGSGGGGSGGGASEVQLVQSGAEVKKPGESLKISCKG |
| | | SGYSFTGYNMNWVRQMPGKGLEWMGNIDPYYGGTTYNRKF |
| | | KGQVTISADKSISTAYLQWSSLKASDTAMYYCARSVGPFDSW |
| | | GQGTLVTVSSCPPCPAPELLGGPSVFLFPPKPKDTLMISRTPE |
| | | VTCVVVDVSHEDPEVKFNWYVDGVEVHNAKTKPREEQYNSTY |
| | | RVVSVLTVLHQDWLNGKEYKCKVSNKALPAPIEKTISKAKGQP |
| | | REPQVYTLPPSRDELTKNQVSLTCLVKGFYPSDIAVEWESNGQ |
| | | PENNYKTTPPVLDSDGSFFLYSKLTVDKSRWQQGNVFSCSVM |
| | | HEALHNHYTQKSLSLSPGK |
| 019008 | 85 | aagcttgccgccatggaagccccagctcagcttctcttcctcctgctactctggctcccag |
| | | ataccaccggagaaattgtgttgacacagtctccagccaccctgtctttgtctccaggcga |
| | | aagagccaccctctcctgccgaacaagtgaaaatgtttacagctacttagcctggtacca |
| | | acagaaacctggccaggctcctaggctcctcatctattttgcaaaaaccttagcagaagg |
| | | aattccagccaggttcagtggcagtggatccgggacagacttcactctcaccatcagca |
| | | gcctagagcctgaagattttgcagtttattactgtcaacatcattccgataatccgtggacat |
| | | tcggccaagggaccaaggtggaaatcaaaggtggcggtggctcgggcggtggtggat |
| | | ctggaggaggtgggaccggtgaggtgcagctggtgcagtctggagcagaggtgaaaa |
| | | agcccggagagtctctgaagatttcctgtaagggatccggttactcattcactggctacaa |
| | | tatgaactgggtgcgccagatgcccgggaaaggcctggagtggatgggcaatattgat |
| | | ccttattatggtggtactacctacaaccggaagttcaagggccaggtcactatctccgccg |
| | | acaagtccatcagcaccgcctacctgcaatggagcagcctgaaggcctcggacaccg |
| | | ccatgtattactgtgcacgctcagtcggccctatggactactggggccgcggcaccctgg |
| | | tcactgtctcctctgatcaggagcccaaatcttctgacaaaactcacacatctccaccgtg |
| | | cccagcacctgaactcctgggtggaccgtcagtcttcctcttccccccaaaacccaagg |
| | | acaccctcatgatctcccggacccctgaggtcacatgcgtggtggtggacgtgagccac |
| | | gaagaccctgaggtcaagttcaactggtacgtggacggcgtggaggtgcataatgcca |
| | | agacaaagccgcgggaggagcagtacaacagcacgtaccgtgtggtcagcgtcctc |
| | | accgtcctgcaccaggactggctgaatggcaaggagtacaagtgcaaggtctccaac |
| | | aaagccctcccagcccccatcgagaaaaccatctccaaagccaaagggcagccccg |
| | | agaaccacaggtgtacaccctgcccccatcccgggatgagctgaccaagaaccaggt |
| | | cagcctgacctgcctggtcaaaggcttctatccaagcgacatcgccgtggagtgggaga |
| | | gcaatgggcagccggagaacaactacaagaccacgcctcccgtgctggactccgac |
| | | ggctccttcttcctctacagcaagctcaccgtggacaagagcaggtggcagcagggga |
| | | acgtcttctcatgctccgtgatgcatgaggctctgcacaaccactacacgcagaagagc |
| | | ctctccctgtctccgggtaaatga |
| 019008 | 86 | MEAPAQLLFLLLLWLPDTTGEIVLTQSPATLSLSPGERATLSCR |
| | | TSENVYSYLAWYQQKPGQAPRLLIYFAKTLAEGIPARFSGSGS |
| | | GTDFTLTISSLEPEDFAVYYCQHHSDNPWTFGQGTKVEIKGGG |
| | | GSGGGGSGGGGASEVQLVQSGAEVKKPGESLKISCKGSGYS |
| | | FTGYNMNWVRQMPGKGLEWMGNIDPYYGGTTYNRKFKGQV |
| | | TISADKSISTAYLQWSSLKASDTAMYYCARSVGPMDYWGRGT |
| | | LVTVSSDQEPKSSDKTHTSPPCPAPELLGGPSVFLFPPKPKDT |
| | | LMISRTPEVTCVVVDVSHEDPEVKFNWYVDGVEVHNAKTKPR |
| | | EEQYNSTYRVVSVLTVLHQDWLNGKEYKCKVSNKALPAPIEKT |
| | | ISKAKGQPREPQVYTLPPSRDELTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPGK |
| 019009 | 87 | aagcttgccgccatggaagccccagctcagcttctcttcctcctgctactctggctcccag |
| | | ataccaccggtgaaattgtgttgacacagtctccagccaccctgtctttgtctccaggcga |
| | | aagagccaccctctcctgccgaacaagtgaaaatgtttacagctacttagcctggtacca |
| | | acagaaacctggccaggctcctaggctcctcatctattttgcaaaaaccttagcagaagg |
| | | aattccagccaggttcagtggcagtggatccgggacagacttcactctcaccatcagca |
| | | gcctagagcctgaagattttgcagtttattactgtcaacatcattccgataatccgtggacat |
| | | tcggccaagggaccaaggtggaaatcaaaggtggcggtggctcgggcggtggtggat |
| | | ctggaggaggtggggctagcgaggtgcagctggtgcagtctggagcagaggtgaaaa |
| | | agcccggagagtctctgaggatttcctgtaagggatccggttactcattcactggctacaa |
| | | tatgaactgggtgcgccagatgcccgggaaaggcctggagtggatgggcaatattgat |
| | | ccttattatggtggtactacctacaaccggaagttcaagggccaggtcactatctccgccg |
| | | acaagtccatcagcaccgcctacctgcaatggagcagcctgaaggcctcggacaccg |
| | | ccatgtattactgtgcacgctcagtcggccctatggactactggggccgcggcaccctgg |
| | | tcactgtctcctctgatcaggagcccaaatcttctgacaaaactcacacatctccaccgtg |
| | | cccagcacctgaactcctgggtggaccgtcagtcttcctcttccccccaaaacccaagg |
| | | acaccctcatgatctcccggacccctgaggtcacatgcgtggtggtggacgtgagccac |
| | | gaagaccctgaggtcaagttcaactggtacgtggacggcgtggaggtgcataatgcca |
| | | agacaaagccgcgggaggagcagtacaacagcacgtaccgtgtggtcagcgtcctc |
| | | accgtcctgcaccaggactggctgaatggcaaggagtacaagtgcaaggtctccaac |
| | | aaagccctcccagcccccatcgagaaaaccatctccaaagccaaagggcagccccg |
| | | agaaccacaggtgtacaccctgcccccatcccgggatgagctgaccaagaaccaggt |
| | | cagcctgacctgcctggtcaaaggcttctatccaagcgacatcgccgtggagtgggaga |
| | | gcaatgggcagccggagaacaactacaagaccacgcctcccgtgctggactccgac |
| | | ggctccttcttcctctacagcaagctcaccgtggacaagagcaggtggcagcagggga |
| | | acgtcttctcatgctccgtgatgcatgaggctctgcacaaccactacacgcagaagagc |
| | | ctctccctgtctccgggtaaatga |
| 019009 | 88 | MEAPAQLLFLLLLWLPDTTGEIVLTQSPATLSLSPGERATLSCR |
| | | TSENVYSYLAWYQQKPGQAPRLLIYFAKTLAEGIPARFSGSGS |
| | | GTDFTLTISSLEPEDFAVYYCQHHSDNPWTFGQGTKVEIKGGG |
| | | GSGGGGSGGGGASEVQLVQSGAEVKKPGESLRISCKGSGYS |
| | | FTGYNMNWVRQMPGKGLEWMGNIDPYYGGTTYNRKFKGQV |
| | | TISADKSISTAYLQWSSLKASDTAMYYCARSVGPMDYWGRGT |
| | | LVTVSSDQEPKSSDKTHTSPPCPAPELLGGPSVFLFPPKPKDT |
| | | LMISRTPEVTCVVVDVSHEDPEVKFNWYVDGVEVHNAKTKPR |
| | | EEQYNSTYRVVSVLTVLHQDWLNGKEYKCKVSNKALPAPIEKT |
| | | ISKAKGQPREPQVYTLPPSRDELTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPGK |
| | 221 | AAGCTTGCCGCCATGGAAGCCCCAGCTCAGCTTCTCTTCCT |
| | | CCTGCTACTCTGGCTCCCAGATACCACCGGAGAGGTGCAG |
| | | CTGGTGCAGTCTGGAGCAGAGGTGAAAAAGCCCGGAGAGT |
| | | CTCTGAAGATTTCCTGTAAGGGCTCCGGTTACTCATTCACTG |
| | | GCTACAATATGAACTGGGTGCGCCAGATGCCCGGGAAAGG |
| | | CCTCGAGTGGATGGGCAATATTGATCCTTATTATGGTGGTA |
| | | CTACCTACAACCGGAAGTTCAAGGGCCAGGTCACTATCTCC |
| | | GCCGACAAGTCCATCAGCACCGCCTACCTGCAATGGAGCA |
| | | GCCTGAAGGCCTCGGACACCGCCATGTATTACTGTGCACG |
| | | CTCAGTCGGCCCTTTCGACTCCTGGGGCCAGGGCACCCTG |
| | | GTCACTGTCTCCTCTGGGGGTGGAGGCTCTGGTGGCGGTG |
| | | GCTCTGGCGGAGGTGGATCCGGTGGCGGCGGATCTGGCG |
| | | GGGGTGGCTCTGAAATTGTGTTGACACAGTCTCCAGCCACC |
| | | CTGTCTTTGTCTCCAGGCGAAAGAGCCACCCTCTCCTGCCG |
| | | AGCAAGTGAAAATGTTTACAGCTACTTAGCCTGGTACCAACA |
| | | GAAACCTGGCCAGGCTCCTAGGCTCCTCATCTATTTTGCAA |
| | | AAACCTTAGCAGAAGGAATTCCAGCCAGGTTCAGTGGCAGT |
| | | GGCTCCGGGACAGACTTCACTCTCACCATCAGCAGCCTAGA |
| | | GCCTGAAGATTTTGCAGTTTATTACTGTCAACATCATTCCGA |
| | | TAATCCGTGGACATTCGGCCAAGGGACCAAGGTGGAAATCA |
| | | AAGGTGATCAGGAGCCCAAATCTTCTGACAAAACTCACACA |
| | | TCTCCACCGTGCCCAGCACCTGAACTCCTGGGTGGACCGT |
| | | CAGTCTTCCTCTTCCCCCCAAAACCCAAGGACACCCTCATG |
| | | ATCTCCCGGACCCCTGAGGTCACATGCGTGGTGGTGGACG |
| | | TGAGCCACGAAGACCCTGAGGTCAAGTTCAACTGGTACGTG |
| | | GACGGCGTGGAGGTGCATAATGCCAAGACAAAGCCGCGGG |
| | | AGGAGCAGTACAACAGCACGTACCGTGTGGTCAGCGTCCT |
| | | CACCGTCCTGCACCAGGACTGGCTGAATGGCAAGGAGTAC |
| | | AAGTGCAAGGTCTCCAACAAAGCCCTCCCAGCCCCCATCGA |
| | | GAAAACCATCTCCAAAGCCAAAGGGCAGCCCCGAGAACCA |
| | | CAGGTGTACACCCTGCCCCCATCCCGGGATGAGCTGACCA |
| | | AGAACCAGGTCAGCCTGACCTGCCTGGTCAAAGGCTTCTAT |
| | | CCAAGCGACATCGCCGTGGAGTGGGAGAGCAATGGGCAGC |
| | | CGGAGAACAACTACAAGACCACGCCTCCCGTGCTGGACTC |
| | | CGACGGCTCCTTCTTCCTCTACAGCAAGCTCACCGTGGACA |
| | | AGAGCAGGTGGCAGCAGGGGAACGTCTTCTCATGCTCCGT |
| | | GATGCATGAGGCTCTGCACAACCACTACACGCAGAAGAGC |
| | | CTCTCCCTGTCTCCGGGTAAATGATCTAGA |
| | 222 | MEAPAQLLFLLLLWLPDTTGEVQLVQSGAEVKKPGESLKISCK |
| | | GSGYSFTGYNMNWVRQMPGKGLEWMGNIDPYYGGTTYNRK |
| | | FKGQVTISADKSISTAYLQWSSLKASDTAMYYCARSVGPFDSW |
| | | GQGTLVTVSSGGGGSGGGGSGGGGSGGGGSGGGGSEIVLT |
| | | QSPATLSLSPGERATLSCRASENVYSYLAWYQQKPGQAPRLLI |
| | | YFAKTLAEGIPARFSGSGSGTDFTLTISSLEPEDFAVYYCQHHS |
| | | DNPWTFGQGTKVEIKGDQEPKSSDKTHTSPPCPAPELLGGPS |
| | | VFLFPPKPKDTLMISRTPEVTCVVVDVSHEDPEVKFNWYVDGV |
| | | EVHNAKTKPREEQYNSTYRVVSVLTVLHQDWLNGKEYKCKVS |
| | | NKALPAPIEKTISKAKGQPREPQVYTLPPSRDELTKNQVSLTCL |
| | | VKGFYPSDIAVEWESNGQPENNYKTTPPVLDSDGSFFLYSKLT |
| | | VDKSRWQQGNVFSCSVMHEALHNHYTQKSLSLSPGK |

### Example 19

### Dose Response of TRU-016 in an Established Subcutaneous Human Tumor (DOHH2) Xenograft Model in SCID Mice

The objective of this experiment was to examine the dose response to treatment with TRU-016 in a model of established subcutaneous human tumor (DOHH2) xenograft model in SCID mice. DOHH2 is a CD20⁺CD37⁺ human B-lymphoblastoid cell line derived from a patient with follicular lymphoma (Kluin-Nelemans et al., Leukemia 5:221-224, 1991). Thus, DOHH2 was derived from a patient with a non-Burkitt's NHL.

Five million DOHH2 cells were injected subcutaneously into the flank of female CB-17SCID mice (Harlan, Somerville, N.J.) at 6.5 weeks of age and at a mean weight of 18.0±0.1 g (ranging from 14.6 to 22.6 g). On day 8 post-tumor inoculation, palpable tumors were apparent in a majority of mice. The tumor-bearing mice were sorted into four groups with equivalent mean tumor volumes (n=14 per group; 2 cages of 5 mice and 1 cage of 4 mice for each group). The day of the sort was defined as day 0. Tumor diameters were determined with a pair of calipers and tumor volumes were calculated using the formula: V=½ [length×(width)²]. The baseline mean tumor volume was 228 mm³, the median baseline tumor size was 224 mm³, and the range was 179 to 284 mm³.

TABLE 9
Reagents for in vivo use.

| | | Percent | | |
|---|---|---|---|---|
| | Source and | Protein of | Cocentration | Preparation for |
| Reagent | Lot No. | Interest | and endotoxin | injection |
| PBS | Gibco, | NA | 1X | NA |
| | 14190 | | Endotoxin <0.03 EU/mg | |
| | Lot No. | | | |
| | 1403805 | | | |
| Human IgG | Sigma, I | Not tested | 10 mg/mL | Diluted in PBS to 1.0 mg/mL |
| (huIgG) | 4506 | | Endotoxin = 10 EU/mg | |
| | Lot No. | | | |
| | 085K7545 | | | |
| TRU-016 | Laureate | 100 | 9.6 mg/mL | Diluted in PBS to 1.0 mg/mL |
| | Lot No. | | Endotoxin = 0.01 EU/mg | for the 200 μg |
| | PURT R1- | | | dose; this material |
| | PLT-AP | | | was diluted 1:2 to |
| | | | | prepare the 100 μg |
| | | | | dose, which was then |
| | | | | serially diluted 1:3 to |
| | | | | prepare the other |
| | | | | dose solutions. |

Tumor-bearing groups of SCID mice were treated on days 0, 4, and 8 via IP injection of 0.2 mL of PBS containing 200 μg of huIgG (negative control) or 200, 100, 30, 10, or 3 μg of TRU-016. The two lowest dose solutions of TRU-016 were prepared on the day of injection to avoid the need to add a carrier protein to the most dilute solutions. Drug solutions were color-coded as described below (see Table 10 below).

TABLE 10
Experimental Design

| | Number of mice, | | | | |
|---|---|---|---|---|---|
| | Route of | | Approimate | | |
| | Injection, and | Dose per | mg/kg | | Cumulative |
| | Days of | injection | per | Cumulative | Dose |
| Group ID | Treatment | (μg) | Injection^{a} | Dose (μg) | (~mg/kg)^{a} |
| huIgG | 14 per group | 200 | 11.1 | 600 | 33 |
| TRU-016 | IP injection | 200 | 11.1 | 600 | 33 |
| 200 | | | | | |
| TRU-016 | Days 0, 4, 8 | 100 | 5.6 | 300 | 16.7 |
| 100 | | | | | |
| TRU-016 | | 30 | 1.7 | 90 | 5.0 |
| 30 | | | | | |
| TRU-016 | | 10 | 0.6 | 30 | 1.7 |
| 10 | | | | | |
| TRU-016 3 | | 3 | 0.2 | 9 | 0.5 |

^{a}Note that huIgG and TRU-016 were delivered in μg per mouse, not in mg/kg. The approximate mg/kg is noted for convenience, and is based on the mean weight (18.0 ± 0.1 g) of mice on day 0. The weight range in this experiment was 14.6 to 22.6 g.

Dose solutions were prepared in similar volumes and the contents of the tubes were noted on removable labels. An investigator who was not treating or assessing the mice placed a color code on each tube and noted the code and identity of the tube contents in a laboratory notebook. Because of the size of the experiments and limitations with regard to technical staff, it was not feasible to randomize the mice completely (such that each cage would contain mice from more than one treatment group) or to have separate investigators perform the treatment and monitoring of mice. The possibility of investigator bias is reduced, but not eliminated, with this design because investigators performing the study were only partially “blinded” in that they did not know which treatment a particular group of mice was receiving, but did know that all the mice within a group of 3 cages belonged to the same group. The code was revealed at the end of the study; however, the investigator who was aware of the code was able to monitor the study results on an interim basis.

Mice were monitored daily by visual inspection. Weights were determined weekly, and tumor diameters were determined at least 3 times per week (M, W, F) by an observer blinded (see above) to the treatment groups. Tumor volumes were calculated as described above. Mice were euthanized if their tumor volume reached more than 1500 mm³ (or 1200 mm³ on Fridays). Death was not an endpoint in the tumor protocols and, unless noted otherwise, “survival” of a mouse was determined by the time it was euthanized due to its tumor volume reaching the predetermined limits. (The protocol called for mice to be euthanized if (1) their tumor volume exceeded the parameters noted above, (2) ulceration of a tumor occurred, (3) the tumor inhibited the mobility of the mouse, and (4) weight loss exceeded 20% of body weight.)

One mouse in the TRU-016 100 μg treatment group was euthanized on day 35 due to weight loss>20%. This mouse had a tumor volume of 266 mm³ at that time, and was treated as censored data for the survival analysis (not euthanized as of day 35 due to tumor growth). For the calculation of tumor-free incidence at the end of the study, this mouse was classified as one that was euthanized during the study due to growth of its tumor (its tumor was growing back at the time of its death). No other mice were found dead and none were euthanized due to weight loss, tumor ulceration, or impaired mobility. No overt signs of toxicity or weight loss were observed in any of the treatment groups (data not shown).

All statistical analyses were performed using GraphPad Prism software. Significant differences in mean tumor volumes and mean relative tumor volumes were determined using a one-way ANOVA for nonparametric data (Kruskal-Wallis test) with Dunn's multiple comparison post test. To examine differences between each of the TRU-016 treated groups and the huIgG group, all groups were compared. For comparisons between the TRU-016 groups only, the huIgG group was excluded. In addition, the high and middle dose (200, 100, and 30 μg) groups were analyzed as a one data set, and the middle and low dose (30, 10, and 3 μg) groups were analyzed as another data set. Significant differences in survival of mice over time were determined using Kaplan-Meier survival analysis with a log-rank test for comparing survival curves. Significant differences in the incidence of tumor-free mice were determined using Fisher's exact test. p values<0.05 were considered significant.

TRU-016 had a dose-dependent inhibitory effect on the growth of DOHH2 tumors. With the exception of the low (3 μg) dose regimen group, the mean tumor volume of each TRU-016 treated group was significantly lower than that of the human IgG treated group as early as day 5, and remained lower through day 12. The huIgG treated mice were euthanized starting on day 12; therefore, comparisons of tumor volumes of the TRU-016 treated groups to the huIgG group were not performed for later time points. In terms of a dose response, there was no significant difference in the mean tumor volumes of the two highest dose groups at any point in the study. In contrast, the mean tumor volumes of these two groups differed significantly from those of each of the three lower dose groups from days 12 through 16 (day 16 was the last evaluable timepoint for the low dose group). Similarly, the mean tumor volumes in mice of the 30 μg and 10 μg dose groups differed from each other and from the low dose group over this same period.

The tumors in the mice treated with huIgG grew rapidly, and all of the mice in this group were euthanized by day 19. As summarized in Tables 11 and 12, the survival of mice treated with any of the TRU-016 dose regimens was prolonged relative to the huIgG treated group (p<0.0001 in all cases). In terms of a dose response, there was no significant difference in the survival curves of mice treated with the highest (200 and 100 μg) dose regimens (p=0.7091). With the exception of this group comparison, there was a significant difference between the survival curve of each dose group and the survival curve of each of the groups treated with a lower dose regimen (p values ranged from 0.0132 to <0.0001).

TABLE 11
Median Survival Time and Incidence of Tumor-Free
Mice at the end of the Observation Period

| | | | Death for | | p Value for |
|---|---|---|---|---|---|
| | | | Reasons | Tumor- | Fischer's |
| | | | Other Than | Free | Exact Test |
| | | Median | Large | Incidence | (comparison |
| Treatment | Cumulative | Survival Time | Tumor | at End of | of tumor-free |
| Group^{a} | Dose | (Days)^{b} | Volume | Study^{c} | incidence)^{d} |
| HuIgG 200 | 600 μg | 14 | 0/14 | 0/14 (0%) | NA |
| TRU-016 | 600 μg | Undefined^{ef} | 0/14 | 11/14 | <0.0001 |
| 200 | | | | (79%)^{g} | |
| TRU-016 | 300 μg | Undefined | 1/14^{h} | 11/14 (79%) | <0.0001 |
| 100 | | | | | |
| TRU-016 30 | 90 μg | 35 | 0/14 | 5/14 (36%) | 0.0407 |
| TRU-016 10 | 30 μg | 28 | 0/14 | 0/14 (0%) | NA |
| TRU-016 3 | 9 μg | 19 | 0/14 | 0/14 (0%) | NA |

^{a}Mice were treated with the indicated protein via IP injection on days 0, 4, and 8. The numbers indicate the amount of protein (in μg) injected per day.
^{b}“Survival” of a mouse was determined by the day it was euthanized due to tumor growth. One mouse in the TRU-016 100 μg dose group was euthanized on day 35 due to >20% weight loss. The mouse had a tumor volume of 266 mm³ at that time, and was treated as censored data (tumor volume did not reach predetermined limit by day 35) for the Kaplan Meier analysis. No other mice were euthanized for reasons other than its tumor volume reaching the predetermined limit.
^{c}“Tumor-free” mice had no palpable SC tumors. The absence of tumor cells was not confirmed by histology. The study ended on day 61.
^{d}Each group was compared with the HuIgG treated control group.
^{e}The median survival time is undefined when >50% of the mice are alive at the end of the observation period.
^{f}Values in bold face indicate that the survival curves of the indicated group are significantly different from those of HuIgG control (p < 0.0001 in each case, log rank test).
^{g}Values in bold face are significantly different from the huIgG treated control group.
^{h}One mouse was euthanized on day 35 due to >20% weight loss. The mouse had a tumor volume of 266 mm³ at that time and was treated as censored data for the Kaplan Meier analysis.

TABLE 12
p Values for Comparison of Survival Curves and
Tumor-Free Incidence Between TRU-016 Treated Groups

| | p Values for Indicated Comparisons | |
|---|---|---|
| | Log rank test | Fisher's exact test |
| | (comparison of survival | (comparison of tumor- |
| Group Comparison^{a} | curves) | free incidence) |
| 200 vs 100 | 0.7091 | 1.0000 |
| 200 vs 30 | 0.0132^{b} | 0.0542 |
| 200 vs 10 | <0.0001 | <0.0001 |
| 200 vs 3 | <0.0001 | <0.0001 |
| 100 vs 30 | 0.0035 | 0.0542 |
| 100 vs 10 | <0.0001 | <0.0001 |
| 100 vs 3 | <0.0001 | <0.0001 |
| 30 vs 10 | 0.0002 | 0.0407 |
| 30 vs 3 | <0.0001 | 0.0407 |
| 10 vs 3 | <0.0001 | NA |

^{a}See legend to Table 11 for information on the groups.
^{b}p values <0.05 are in bold face for emphasis.

All of the mice in the huIgG treated group and in the two lowest (10 and 3 μg) TRU-016 dose groups were euthanized due to growth of their tumors. In contrast, the majority of tumors in the groups of mice treated with 200 or 100 μg of TRU-016 regressed to the point that no palpable tumor was present. By the end of the study, 11/14 (79%) of the mice in each of the two highest dose groups and 5/14 (36%) of the mice in the 30 μg dose group remained tumor-free (p<0.0001 and 0.0407, respectively, vs. huIgG group).

Thus, TRU-016 exhibited dose-dependent inhibitory effects on the growth of established subcutaneous human tumor (DOHH2) xenografts in SCID mice. The two highest dose regimens [100 or 200 μg per IP injection; cumulative dose of 300 or 600 μg (˜16.7 or 33 mg/kg, respectively)] had similar inhibitory effects and were the most efficacious of the regimens tested in terms of inhibiting tumor growth, prolonging survival, and inducing complete tumor regression.

### Example 20

### Efficacy of TRU-016 and Rituxan as Single Agents in an Established Human Tumor (DOHH2) Xenograft Model in SCID Mice

The objective of this study was to examine the efficacy of TRU-016 and Rituxan as single agents in a model of established human tumor (DOHH2) xenografts in SCID mice. As set out above, DOHH2 is a CD20⁺CD37⁺ human B-lymphoblastoid cell line derived from a patient with follicular lymphoma.

Five million DOHH2 cells were injected subcutaneously into the flank of female CB-17SCID mice (Harlan, Somerville, N.J.) at 6.5 weeks of age. On day 8 post-tumor inoculation, palpable tumors were apparent in a majority of the mice. The tumor-bearing mice were sorted into four groups (n=15 per group; 3 cages of 5 mice for each group) with equivalent mean tumor volumes. The day of the sort was defined as day 0 of the study. Tumor diameters were determined with a pair of calipers and tumor volumes were calculated using the formula: V=½ [length×(width)²]₃. The baseline mean tumor volume was 228 mm³; the median baseline tumor size was 227 mm; and the range was 181 to 272 mm³. Mice (15 per treatment group) were treated on days 0, 4, and 8 via IP injection of 0.2 mL of PBS containing 200 μg human IgG, TRU-016, or Rituxan (for a total of 600 μg after the three treatments).

For the huIgG, TRU-016, and Rituxan IP treated groups, solutions were prepared in similar volumes and the contents of the tubes were noted on removable labels. An investigator who was not treating or assessing the mice placed a color code on each tube and noted the code and identity of the tube contents in a laboratory notebook. Because of the size of the experiments and limitations with regard to technical staff, it was not feasible to randomize the mice completely (such that each cage would contain mice from more than one treatment group) or to have separate investigators perform the treatment and monitoring of mice. The possibility of investigator bias is reduced, but not eliminated, with this design because investigators performing the study were only partially “blinded” in that they did not know which treatment a particular group of mice was receiving, but did know that all the mice within a group of 3 cages belonged to the same group. The code was revealed at the end of the study; however, the investigator who was aware of the code was able to monitor the study results on an interim basis.

Mice were monitored daily by visual inspection. Weights were determined weekly, and tumor diameters were determined at least 3 times per week (M, W, F) by an observer blinded (see above) to the treatment groups. Tumor volumes were calculated as described above. Tumor volumes on the last day that all mice were alive in each group were also expressed in terms of tumor volumes relative to day 0, using the formula:

$$Relative tumor volume on day of interest = \frac{( \begin{matrix}volume on day of interest - \\ volume on day 0\end{matrix} )}{volume on day 0}$$

Mice were euthanized if their tumor volume reached more than 1500 mm3 (or 1200 mm3 on Fridays). Death is not an endpoint in our tumor protocols, and unless noted otherwise, “survival” of a mouse was determined by the time it was euthanized due to its tumor volume reaching the predetermined limits. (Our protocol calls for mice to be euthanized if their tumor volume exceeds the parameters noted above, ulceration of a tumor occurs, the tumor inhibits the mobility of the mouse, or if weight loss exceeds 20%.)

All statistical analyses were performed using GraphPad Prism software. Significant differences in mean tumor volumes and mean relative tumor volumes were determined using a one-way ANOVA for nonparametric data (Kruskal-Wallis test) with Dunn's multiple comparison post test. Significant differences in survival of mice over time were determined using Kaplan-Meier survival analysis with a log-rank test for comparing survival curves. Significant differences in the incidence of tumor-free mice were determined using Fisher's exact test. p values<0.05 were considered significant.

Mice were euthanized when their tumor volume reached the limits described above. One mouse in the TRU-016 treatment group was euthanized on day 45 due to weight loss>20%. This mouse had no apparent SC tumor at that time, and was treated as censored data for the survival analysis (not euthanized as of day 45 due to tumor growth) and was not included in the comparison of tumor-free incidence at the end of the study. No other mice were found dead and none were euthanized due to weight loss, tumor ulceration, or impaired mobility. No overt signs of toxicity or weight loss were observed in any of the treatment groups (data not shown).

The TRU-016 and Rituxan treated mice exhibited a rapid response to treatment. Mean tumor volumes of the TRU-016- and Rituxan-treated groups were significantly lower than that of the human IgG treated group as early as day 4 (after a single injection of drug) and remained lower through day 11. There were no significant differences in mean tumor volumes or mean relative tumor volumes between the TRU-016 and Rituxan treated groups through day 11. The huIgG treated mice were euthanized starting on day 11; therefore, comparisons of tumor volumes were not performed for later time points.

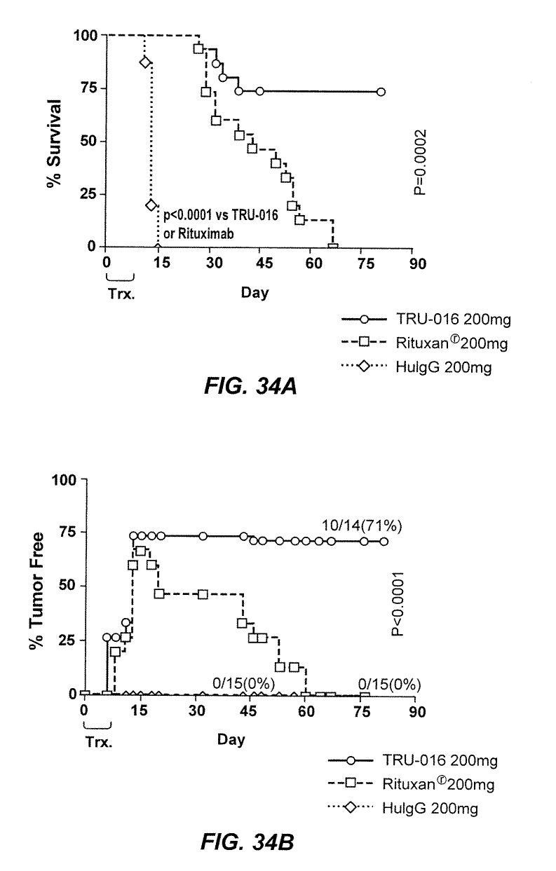

The tumors in the mice treated with huIgG grew rapidly and all mice in this group were euthanized by day 15. In contrast, by day 15, the majority of tumors in the TRU-016 and Rituxan treated groups had regressed to the point that no palpable tumor was present. Notably, the response to treatment was durable only in the TRU-016 treated group. By the end of the study, all of the Rituxan-treated mice were euthanized due to growth of their tumors, whereas 10/14 (71%) of the mice in the TRU-016 treated group remained tumor-free. See Table 13. Thus, at the end of the study, the survival curves and the incidence of tumor-free mice in the TRU-016 treated group differed significantly from the huIgG control group and the Rituxan treated group. FIG. 34 shows that TRU-016 was statistically superior to Rituxan in the in vivo treatment of this animal model of follicular lymphoma.

TABLE 13
Median Survival Time and Incidence of Tumor-Free
Mice at the end of the Observation Period

| | | | p Value | Death for | | Fischer's |
|---|---|---|---|---|---|---|
| | Treatment | | from | Reasons | Tumor- | Exact Test |
| | Days and | Median | Log | Other Than | Free | (comparison |
| Treatment | Cumulative | Survival | Rank | Tumor Size | Mice at | of tumor-free |
| Group | Dose | Time (Days)^{a} | Test^{b} | Sacrifice | Day 81^{c} | incidence)^{b} |
| HuIgG | Days 0, 4, 8 | 13 | — | 0/15 | 0/15 (0%) | NA |
| | 600 μg | | | | | |
| TRU-016 | Days 0, 4, 8 | Undefined^{de} | <0.0001 | 1/15^{f} | 10/14 | <0.0001 |
| IP | 600 μg | | | | (71%)^{f} | |
| Rituxan IP | Days 0, 4, 8 | 43 | <0.0001 | 0/15 | 0/15 (0%) | NA |
| | 600 μg | | | | | |

^{a}“Survival” of a mouse was determined by the day it was euthanized due to tumor growth. One mouse in the TRU-016 dose group was euthanized on day 45 due to >20% weight loss. The mouse had no apparent SC tumor at that time, and was treated as censored data (tumor volume did not reach predetermined limit by day 45) for the Kaplan Meier analysis. No other mice were euthanized for reasons other than its tumor volume reaching the predetermined limit.
^{b}Each group was compared with the HuIgG treated control group.
^{c}“Tumor-free” mice had no palpable SC tumors. The absence of tumor cells was not confirmed by histology.
^{d}The median survival time is undefined when >50% of the mice are alive at the end of the observation period.
^{e}Bold-faced values are significantly different from those of HuIgG control.
^{f}One mouse was euthanized on day 45 due to >20% weight loss. The mouse had no apparent SC tumor at that time and was excluded from the group for the comparison of tumor-free mice at day 81.

In conclusion, TRU-016 and Rituxan were efficacious as single agents in a human tumor (DOHH2) xenograft model in SCID mice. While both agents caused an initial tumor regression in the majority of mice, long-term tumor regression was observed only in the group of mice treated with TRU-016 as tumors relapsed after optimal anti-CD20 treatment. Consequently, TRU-016, a humanized anti-CD37 SMIP, shows significant efficacy in pre-clinical tumor xenograft models including models that show that Rituxan treatment fails over time. These results therefore suggest that TRU-016 treatment of B cell lymphoma and leukemia patients is beneficial and is a viable alternative treatment in patients who fail Rituxan treatment.

### Example 21

### In Vitro Evaluation of Combination Effects of TRU-016 with Chemotherapeutic Agents

The data shown in Example 15 demonstrated that TRU-016 acts synergistically in combination with the chemotherapeutic agent fludarabine to kill chronic lymphocytic leukemia (CLL) cells in vitro. As CLL cells do not actively divide in cell culture in vitro, the data indicate that cell proliferation is not required for the pro-apoptotic effect of TRU-016 for its synergy with chemotherapeutic agents. The purpose of this study, therefore, was to determine whether TRU-016 and various chemotherapeutic agents were effect on a mantle cell lymphoma (MCL) cell line, Rec-1, that actively grows and divides in cell culture in vitro. whether the combination of TRU-016 and a chemotherapeutic agent (drug) would desensitize or enhance the response of mantle cell lymphoma cells to various chemotherapeutic agents. The chemotherapeutic agents tested were doxorubicin, vincristine, and fludarabine, which are used to treat non-Hodgkin's lymphoma and other lymphoid malignancies.

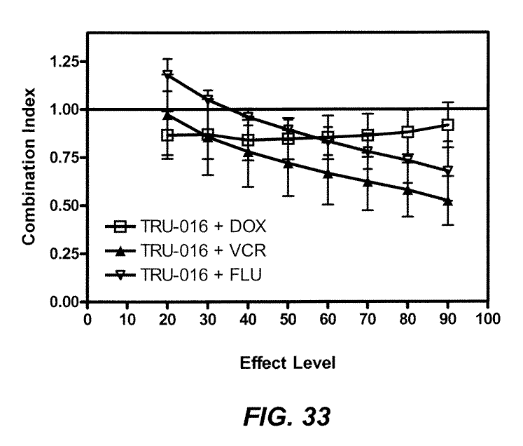

Rec-1 cells, a CD37+ human B cell line established from a patient with mantle cell lymphoma, were tested for growth inhibition in response to crosslinked TRU-016 in the presence or absence of doxorubicin, vincristine, or fludarabine (see FIG. 33). TRU-016 was preincubated with anti-human IgG F(ab)′₂ to crosslink the protein. Cells were cultured with medium alone or with medium containing various concentrations of the crosslinked TRU-016 protein, in the presence or absence of various concentrations of doxorubicin, vincristine, or fludarabine. Cultures were incubated for 96 hours and growth inhibition was assessed using an ATP viable cell detection system (i.e., viable cells quantified by ATP release).

The Median Effect/Combination Index (CI) method of Chou and Talalay (Adv. Enzyme Regul. 22:27-55, 1984) was used for data analysis. A numerical value, assigned to each drug combination at predefined dose levels, enabled quantitative drug/drug interaction comparisons between different drug combinations. Results were expressed as combination indices (CI) vs. effect level, in which effect level represented percent inhibition of cell growth. The mean CI±SEM for each effect level was averaged over three experiments. A CI<1.0 was considered synergy, CI=1.0 additivity, and CI>1.0 antagonism. Values presented are the mean ±SEM for each effect level, averaging three independent assays.

None of TRU-016 and chemotherapeutic agent combinations were antagonistic (CI>1.0) across all effect levels. The combination of TRU-016 with vincristine or fludarabine was synergistic (CI<1.0) and the combination of TRU-016 and doxorubicin was additive (CI not significantly different from 1.0).

Therefore, the combination of TRU-016 with each of the three chemotherapeutic agents tested did not desensitize target cells to drug-induced growth inhibition, but instead resulted in synergistic or additive inhibitory effects on target cell growth. These data indicate that the efficacy of established chemotherapeutics increase when used in combination with TRU-016.

### Example 22

### TRU-016 in the Treatment of Refractory B Cell Diseases

Additional therapies for B lymphoma and leukemia are needed for patients who fail or relapse with current standard of care. The objective of this study is to examine the dose response to treatment with TRU-016 in a Phase ½ study of patients with previously-treated B cell chronic lymphocytic leukemia (CLL), small lymphocytic lymphoma (SLL) and non-Hodgkins lymphoma (NHL).

The thrice weekly IV administration of TRU-016 for a two week period of time has already been shown to be well tolerated by cynomologous monkeys at doses of 0.5 mg/kg, 5 mg/kg, and from 10 mg/kg to 50 mg/kg. In addition, doses of 5 mg/kg and 50 mg/kg have been shown to be well tolerated in rats. Moreover, variable doses have been shown to be well tolerated in mice as shown in the in vivo studies described herein. Thus, in the initial open label dose escalation phase of the human study, TRU-016 (at doses of 0.3, 1.0, 3.0, and 10.0 mg/kg) is administered intravenously once weekly for 4 weeks in cohorts of about 3-6 patients each. TRU-016 drug product (40 mg/vial) is provided as a sterile, preservative-free liquid. Each single-use vial of TRU-016 contains 4 mL of TRU-016 (10 mg/mL) in an aqueous formulation buffer (20 mM sodium acetate, 50 mM glycine, and 190 mM sucrose) at pH 6.0. The container/closure system is a 10 mL glass serum vial, with a 20 mm stopper, sealed with an aluminum crimp with a plastic tamper-evident flip-off cap to maintain stability, sterility, and safety of the drug product. Safety and toxicity is monitored in all patients. Patients undergo physical examinations and serial blood monitoring for TRU-016 levels and TRU-016 antibody formation.

Subsequent cohorts may receive 3 doses of TRU-016 at 3.0 or 10.0 mg/kg during the first week followed by weekly dosing for three additional weeks. In the second phase of the study, an expanded cohort of patients will be treated at the highest, best tolerated dose from the dose escalation portion of the study.

Subjects will undergo clinical re-staging on day 29 that will be repeated about two months later (including bone marrow biopsy and aspirate in patients with partial response (PR) or better, and then every three months after until there is progression of the disease, withdrawal from the study, completion of two years of follow up evaluations or death.

### Example 23

### TRU-016 in the Treatment of Relapse or in Rituximab-Refractory Disease

Additional therapies for patients who relapse or who develop rituximab-refractory disease are needed, because despite high initial response rates of lymphomas treated with CD20-targeted therapies, tumor regression is generally not durable and disease relapse is common in years following completion of treatment.

Conditions where CD20-targeted therapies fail and where TRU-016 may be particularly useful include (i) treatment of tumors refractory to initial CD20 therapy, (ii) treatment of tumors with intermediate sensitivity, i.e., producing partial regression or delay in tumor progression in response to CD20-targeted therapies, and (iii) treatments that produce apparent complete regressions or cures, but that ultimately are not durable and relapse. The second condition is exemplified in the DoHH2 model shown in Examples 19 and 20.

DoHH2 tumors initially respond to rituximab treatment but regrow following cessation of treatment. Notably, TRU-016 treatment in these models produces tumor regressions with superior, more durable responses following cessation of treatment. Therefore, on the basis of data already obtained (including Table 13 and FIG. 34), it is contemplated that the treatment of rituximab-refractory tumors or relapsing tumors with TRU-016 may be particularly effective.

To demonstrate this in a model of rituximab failure, the study described in Example 20 is repeated with modifications as set out herein. Established subcutaneous xenograft tumors of the mantle cell lymphoma DoHH2 are treated with a high dose (100 mg) of rituximab. This dose has been shown to be sufficient to induce significant regression, but not produce durable responses or cures. Following relapse and tumor regrowth, animals are treated with an equivalent dose of TRU-016 and its efficacy in inducing tumor regression or elimination is measured over time.

Additionally, it is known that following patient treatment with rituximab, some drug may remain in the tissue or on the surface of refractory tumor cells for extended periods of time. On the basis of the observed combined activity of rituximab and TRU-016, it is expected that the re-treatment of residual tumor with TRU-016 following failed treatment with rituximab will produce synergistic antitumor activity with residual, tumor-bound rituximab.

With advances in predictive medicine, sets of biological markers may become sufficiently characterized so as to predict tumor responsiveness to CD20-targeted therapies. In the event that biological markers predict against tumor response to CD20-targeted therapies, even though the tumors express CD20, the invention contemplates that treatment with CD37-specific binding polypeptides would be indicated.

Numerous modifications and variations in the invention as set forth in the above illustrative examples are expected to occur to those skilled in the art. Consequently, only such limitations as appear in the appended claims should be placed on the invention.

What is claimed is:

1. A method of treating a non-Burkitt's B cell malignancy, comprising administering to an individual in need thereof one or more CD37-specific binding molecules.

2. The method of claim 1 wherein one or more CD37-specific binding molecules is a polypeptide comprising complementarity-determining regions from a CD37-specific antibody.

3. The method of claim 2 wherein one or more CD37-specific binding molecules is a CD37-specific SMIP.

4. The method of claim 3 wherein the CD37-specific SMIP is a humanized CD37-specific SMIP.

5. The method of claim 4 wherein the humanized CD37-specific SMIP is TRU-016.

6. The method of claim 4 wherein the humanized CD37-specific SMIP is a polypeptide that exhibits at least 80 percent identity to the polypeptide set forth in SEQ ID NO: 2, wherein the humanized CD37-specific SMIP polypeptide binds CD37.

7. The method of claim 4 wherein the humanized CD37-specific SMIP is a polypeptide comprising an amino acid sequence selected from the group consisting of SEQ ID NO: 6, 8, 10, 12, 14, 16, 18, 20, 22, 24, 26, 28, 30, 32, 34, 36, 38, 40, 42, 44, 46, 48, 50, 52, 54, 56, 58, 60, 80, 82, 84, 86, 88, and 222.

8. The method of claim 7 wherein the humanized CD37-specific SMIP is a polypeptide comprising an amino acid sequence set forth in SEQ ID NO: 222.

9. The method of claim 4 wherein the humanized CD37-specific SMIP polypeptide is a polypeptide that comprises a CDR1, a CDR2, and a CDR3, that exhibits at least 80 percent identity to the polypeptide set forth in SEQ ID NO: 2.

10. The method of claim 4 wherein the polypeptide further comprises a human framework domain separating each of CDR1, CDR2, and CDR3.

11. The method of claim 4 wherein the humanized CD37-specific SMIP polypeptide binds CD37 and comprises a hinge region polypeptide comprising an amino acid sequence selected from the group consisting of SEQ ID NOS: 90, 92, 94, 96, 98, 100, 102, 104, 106, 108, 110, 112, 114, 115, 116, 118, 120, 122, 124, 126 and 127.

12. The method of claim 4 wherein the humanized CD37-specific SMIP polypeptide binds CD37 and comprises a linker comprising (Gly₄Ser)ₙ, wherein n is 1, 2, 3, 4, 5, or 6.

13. The method of claim 9 wherein the CDR1 of the light chain comprises the amino acid sequence selected from the group consisting of SEQ ID NOS: 128 (RTSQNVYSYLA), 129 (RTSESVYSYLA), 130 (RASQSVYSYLA), 131 (RASQSVSSYLA) and 132 (RASQSVSYYLA).

14. The method of claim 9 wherein the CDR1 of the heavy chain comprises the amino acid sequence selected from the group consisting of SEQ ID NOS: 133 (SYMNM) and 134 (SYWIG).

15. The method of claim 9 wherein the CDR2 of the light chain comprises the amino acid sequence selected from the group consisting of SEQ ID NOS: 135 (AASSLQS), 136 (GASTRAT) and 137 (DASNRAT).

16. The method of claim 9 wherein the CDR2 of the heavy chain comprises the amino acid sequence selected from the group consisting of SEQ ID NOS: 138 (IIYPGDSDTRYSPSFQG) and 139 (RIDPSDSYTNYSPSFQG).

17. The method of claim 9 wherein the CDR3 of the light chain comprises the amino acid sequence of SEQ ID NO: 220 (QHHSDNPWT).

18. The method of claim 9 wherein the CDR3 of the heavy chain comprises the amino acid sequence selected from the group consisting of SEQ ID NOS: 211 (SVGPMDY), 212 (SVGPFDY), 213 (SVGPMDV), 214 (SVGPFDS), 215 (SVGPFDP), 216 (SVGPFQH), 217 (SVGPFDV), 218 (SVGPFDI) and 219 (SVGPFDL).

19. The method of claim 4 wherein the humanized CD37-specific SMIP polypeptide is a polypeptide that comprises an FR1, an FR2, an FR3, and an FR4.

20. The method of claim 19 wherein the FR1 of the light chain comprises the amino acid sequence selected from the group consisting of SEQ ID NOS: 170-181.

21. The method of claim 19 wherein the FR1 of the heavy chain comprises the amino acid sequence selected from the group consisting of SEQ ID NOS: 140-146.

22. The method of claim 19 wherein the FR2 of the light chain comprises the amino acid sequence selected from the group consisting of SEQ ID NOS: 182-193.

23. The method of claim 19 wherein the FR2 of the heavy chain comprises the amino acid sequence selected from the group consisting of SEQ ID NOS: 147-153.

24. The method of claim 19 wherein the FR3 of the light chain comprises the amino acid sequence selected from the group consisting of SEQ ID NOS: 194-205.

25. The method of claim 19 wherein the FR3 of the heavy chain comprises the amino acid sequence selected from the group consisting of SEQ ID NOS: 154-160.

26. The method of claim 19 wherein the FR4 of the light chain comprises the amino acid sequence selected from the group consisting of SEQ ID NOS: 206-210.

27. The method of claim 19 wherein the FR4 of the heavy chain comprises the amino acid sequence selected from the group consisting of SEQ ID NOS: 161-169.

28. The method of claim 1 further comprising the administration of an additional agent.

29. The method of claim 28 wherein the additional agent is a cytokine, a chemokine, a growth factor, a chemotherapeutic agent, or a radiotherapeutic agent.

30. The method of claim 1 wherein the non-Burkitt's B cell malignancy is a B-cell chronic lymphocytic leukemia/small lymphocytic lymphoma, a B-cell prolymphocytic leukemia, an acute lymphoblastic leukemia (ALL), a lymphoplasmacytic lymphoma, a marginal zone lymphoma, a hairy cell leukemia, a plasma cell myeloma/plasmacytoma, a follicular lymphoma, mantle cell lymphoma, a diffuse large cell B-cell lymphoma, a transforming large B cell lymphoma, a mediastinal large B-cell lymphoma, an intravascular large B-cell lymphoma, a primary effusion lymphoma, or a non-Burkitt's non-Hodgkins lymphoma (NHL).

31. The method of claim 30 wherein the marginal zone lymphoma is a splenic marginal zone B-cell lymphoma, a nodal marginal zone lymphoma, or an extranodal marginal zone B-cell lymphoma of mucosa-associated lymphoid tissue type lymphoma.

32. The method of claim 30 wherein the non-Burkitt's B cell malignancy is a chronic lymphocytic leukemia (CLL).

30. The method of claim 30 wherein the non-Burkitt's B cell malignancy is an acute lymphoblastic leukemia (ALL).

31. The method of claim 30 wherein the non-Burkitt's B cell malignancy is a follicular lymphoma.

32. A humanized CD37-specific SMIP polypeptide comprising an amino acid sequence set forth in SEQ ID NO: 222.

33. An isolated nucleic acid molecule that encodes a humanized CD37-specific SMIP polypeptide comprising an amino acid sequence set forth in SEQ ID NO: 222.

34. An isolated nucleic acid molecule comprising the nucleotide sequence set forth in SEQ ID NO: 221.

35. A vector comprising the nucleic acid molecule of claim 33 or 34.

36. A host cell comprising the vector of claim 35.

37. A process of producing a polypeptide comprising culturing the host cell of claim 36 under suitable conditions to express the polypeptide, and optionally isolating the polypeptide from the culture.

38. A composition comprising the humanized CD37-specific SMIP polypeptide of claim 32 and a pharmaceutically acceptable carrier.

39. The method of any one of claims 1-31 wherein the CD37-specific SMIP or CD37-specific binding molecule comprises an amino acid sequence consisting of SEQ ID NO: 222.

40. A kit for treating a non-Burkitt's B cell malignancy comprising:
(a) the composition of claim 38; and
(b) a protocol for using the kit to reduce non-Burkitt's malignant B cells.

41. The kit of claim 40 further comprising a cytokine, a chemokine, a growth factor, a chemotherapeutic agent, or a radiotherapeutic agent.
